(19) United States
(12) Patent Application Publication
(10) Pub. No.: US 2024/0374739 A1
(43) Pub. Date: Nov. 14, 2024

(54) BILE ACID CONJUGATE

(71) Applicant: Curtin University, Bentley, Western Australia (AU)

(72) Inventors: Hani Al-Salami, Bentley, Western Australia (AU); Armin Mooranian, Bentley, Western Australia (AU); Giuseppe Luna, Bentley, Western Australia (AU); Anton Dolzhenko, Bentley, Western Australia (AU); John Mamo, Bentley, Western Australia (AU); Ryu Takechi, Bentley, Western Australia (AU); Virginie Lam, Bentley, Western Australia (AU)

(21) Appl. No.: 18/718,106

(22) PCT Filed: Dec. 9, 2022
(86) PCT No.: PCT/AU2022/051481

§ 371 Date: Jun. 10, 2024

(30) Foreign Application Priority Data
Dec. 10, 2021 (AU) 2021904008

(51) Int. Cl. A61K 47/54; A61P 25/16; A61P 25/28

(52) U.S. Cl. CPC A61K 47/554; A61P 25/16; A61P 25/28

(57) ABSTRACT

The present invention relates to compounds having a range of beneficial therapeutic properties and effects, including neuroprotective effects, anti-inflammatory activity, antioxidative properties, and epithelial cell protective effects. These properties enable the compounds to be useful for treating or preventing a wide range of disorders and conditions. The disorders and conditions include neurological disorders (including neurodegenerative conditions such as Alzheimer's disease, dementia caused by Alzheimer's disease, Parkinson's disease, amyotrophic lateral sclerosis (motor neurone disease), multiple sclerosis and brain injury), the cognitive decline associated with these neurological disorders (such as memory loss), and disorders caused by stress-induced cellular damage in the inner or middle ear of a subject (such as vestibular disorders, hearing impairment, and conditions related to hair cell degeneration or hair cell death).

The present application claims priority from Australian provisional patent application no. 2021904008 filed 10 Dec. 2021. The entire contents of Australian provisional patent application no. 2021904008 are incorporated herein by reference.

### TECHNICAL FIELD

The present invention relates to compounds having a range of beneficial therapeutic properties and effects, including neuroprotective effects, anti-inflammatory activity, antioxidative properties, and epithelial cell protective effects. These properties enable the compounds to be useful for treating or preventing a wide range of disorders and conditions.

The disorders and conditions include neurological disorders (including neurodegenerative conditions such as Alzheimer's disease, dementia caused by Alzheimer's disease, Parkinson's disease, amyotrophic lateral sclerosis (motor neuron disease), multiple sclerosis, and brain injury), the cognitive decline associated with these neurological disorders (such as memory loss), and disorders caused by stress-induced cellular damage in the inner or middle ear of a subject (such as vestibular disorders, hearing impairment, and conditions related to hair cell degeneration or hair cell death).

### BACKGROUND

Probucol (2,6-ditert-butyl-4-[2-(3,5-ditert-butyl-4-hydroxyphenyl)sulfanylpropan-2-ylsulfanyl]phenol) is a hydrophobic compound that has been widely prescribed as a lipid-lowering or anti-hypercholesteremia drug which is taken orally. Probucol is a potent anti-oxidant drug that has been in clinical use during the past few decades for the treatment and prevention of cardiovascular diseases. The reported common side effects of probucol include bloating, diarrhea, nausea and vomiting, stomach pain, dizziness or fainting, and fast or irregular heartbeat. Headache and numbness or tingling of fingers, toes, or face are also reported as side effects of probucol. Probucol is no longer available in many countries due to concerns in relation to its efficacy and adverse and undesirable effects.

Succinobucol is a monosuccinate derivative of probucol and has anti-inflammatory and antioxidant properties. Succinobucol has been investigated for use in the treatment of atherosclerotic disease, coronary artery disease, diabetes mellitus (type 2 diabetes), and in-stent restenosis. Relative to treatment with placebo, succinobucol has been reported to increase LDL (bad) cholesterol and systolic blood pressure, and to decrease HDL (good) cholesterol.

It would be advantageous to provide new derivatives of probucol and succinobucol which retain the useful properties and beneficial therapeutic effects of these drugs, but result in fewer or reduced side effects typically associated with these drugs.

All references, including any patents or patent applications cited in this specification, are hereby incorporated by reference.

It will be understood that, although a number of prior art publications are referred to herein, this reference does not constitute an admission that any of these publications form part of the common general knowledge in the art, in Australia or in any other country.

### SUMMARY

In various aspects, there is provided the following.

In one aspect, there is provided a compound of Formula (I):

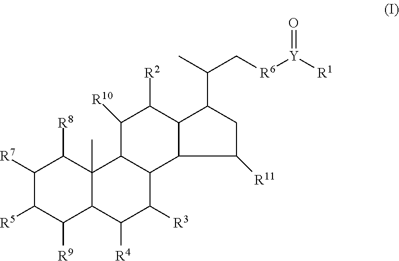

wherein:
R¹ is:

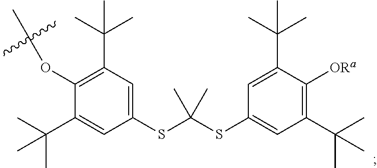

each R², R³, R⁴, R⁵, R⁷, R⁸, R⁹, R¹⁰ and R¹¹ is independently selected from H, substituted or unsubstituted C₁₋₃₀ acyloxy, substituted or unsubstituted benzoyloxy, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, substituted or unsubstituted aryl, substituted or unsubstituted arylalkyl, substituted or unsubstituted heteroaryl, an amino acid moiety, or GL, wherein GL is independently selected from OL, SL, PL₂, CL₃, or NL₂; wherein when R², R³, R⁴, R⁵, R⁷, R⁸, R⁹, R¹⁰ or R¹¹ is substituted, the substituent is independently selected from OH, F, SH, ═O, ═S, Cl, Br, SC₁₋₆ alkyl, C₁₋₆ alkyl or C₁₋₆ alkoxy;
each L is independently selected from H, a metal ion, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, a substituted or unsubstituted benzyl radical, —CH₂CO₂H, or —(CH₂)₂SO₃H; wherein when L is substituted, the substituent is independently selected from OH, SH, ═O, ═S, F, Cl, Br, SC₁₋₆ alkyl, C₁₋₆ alkyl or C₁₋₆ alkoxy;
R⁶ is —(CH₂)ₙ— wherein n is 0 to 12, —CH₂C(═O)NHCH₂—, or —CH₂C(═O)NHCH₂CH₂—;
Y is —C— or —S(═O)—; and
R^{a} is —H or —C(═O)CH₂CH₂COOH;
or a stereoisomer thereof, or a pharmaceutically acceptable salt thereof.

The compounds of Formula (I) have neuroprotective effects, anti-inflammatory activity, antioxidative properties, and epithelial cell protective effects. It is hypothesised that these useful and beneficial properties may be due to the phenolic structure of the compounds. The compounds of Formula (I) are therefore useful in the treatment or prevention of neurological disorders (including neurodegenerative conditions such as Alzheimer's disease, dementia caused by Alzheimer's disease, Parkinson's disease, amyotrophic lateral sclerosis (motor neurone disease), multiple sclerosis, and brain injury), the cognitive decline associated with these neurological disorders (such as memory loss), and disorders caused by stress-induced cellular damage in the inner or middle ear of a subject (such as vestibular disorders, hearing impairment, and conditions related to hair cell degeneration or hair cell death).

In a further aspect, there is provided a pharmaceutical composition comprising a compound of Formula (I) or a stereoisomer thereof, or a pharmaceutically acceptable salt thereof, and a pharmaceutically acceptable carrier, adjuvant or diluent.

In a further aspect, there is provided a method of treating or preventing a neurological disorder or cognitive decline associated with a neurological disorder in a subject, the method comprising administering to the subject an effective amount of a compound of Formula (I) or a stereoisomer thereof, or a pharmaceutically acceptable salt thereof. The neurological disorder may be Alzheimer's disease, dementia caused by Alzheimer's disease, Parkinson's disease, amyotrophic lateral sclerosis (motor neurone disease), multiple sclerosis, or brain injury. Cognitive decline includes memory loss.

In a further aspect, there is provided a method for preventing, reducing or treating the incidence and/or severity of a disorder caused by stress-induced cellular damage in the middle or inner ear of a subject, the method comprising administering to the subject a therapeutically effective amount of a compound of Formula (I) or a stereoisomer thereof, or a pharmaceutically acceptable salt thereof. The disorders caused by stress-induced cellular damage in the inner or middle ear may be vestibular disorders, hearing impairment, or conditions related to hair cell degeneration or hair cell death.

In a further aspect, there is provided a method of producing a compound of Formula (I), comprising coupling probucol or succinobucol to the C17 of a bile acid or derivative thereof.

### BRIEF DESCRIPTION OF THE FIGURES

Embodiments of the present invention will be further described, by way of example only, with reference to the accompanying figures. In the accompanying figures, *p<0.05, **p<0.001, ***p<0.0001, and ns=non-significant. The accompanying figures are described below:

FIG. 1: shows a cytotoxicity study by MTT cell viability assay. AM-MSCs were incubated with different concentrations of (A) probucol, (B) SPQ, (C) HA-1 and, (D) DMSO for 24 hours. D1=DMSO control for 10 μM, D2=DMSO control for 20 μM (n=3).

FIG. 2: shows an estimation of LDH release. Neurodegenerated cells were incubated with probucol (20 μM), SPQ (10 μM), HA-1 (10 μM), and rivastigmine (2 μM) for 24 hours (n=3).

FIG. 3: shows the effect of probucol, SPQ, HA-1 and rivastigmine on: (A) Acetylcholinesterase (AChE) activity and (B) Acetylcholine release in an Alzheimer's disease model using the Amplex Red reagent-based assay. Neurodegenerated cells were incubated with probucol (20 μM), SPQ (10 μM), HA-1 (10 μM) and rivastigmine (2 μM) for 24 hours (n=3).

FIG. 4: shows the measurement of mitochondrial membrane potential by TMRE assay. Neurodegenerated cells were incubated with probucol (20 μM), SPQ (10 μM), HA-1 (10 μM) and rivastigmine (2 μM) for 24 hours (n=3).

FIG. 5: shows gene expression analysis by qRT-PCR for neuronal, cholinergic and AD associated markers (A) BACE1, (B) ChAT, (C) Synapsin 1, and (D) Neuropilin (n=3).

FIG. 6: shows the effect of probucol, SPQ, HA-1 and rivastigmine on (A) intracellular reactive oxidative stress and (B) nitric oxide.

FIG. 7: shows the effect of probucol, SPQ, HA-1 and rivastigmine on glutathione activity.

FIG. 8: shows the results of a cytotoxicity study by MTT cell viability assay. AM-MSCs were incubated with different concentrations of (A) probucol, (B) SPQ, (C) HA-1 and, (D) DMSO for 24 hours. D1=DMSO control for 10 μM, D2=DMSO control for 20 μM (n=3).

FIG. 9: shows the measurement of mitochondrial membrane potential by TMRE assay. Neurodegenerated cells were incubated with probucol (20 μM), SPQ (10 μM), HA-1 (10 μM) and L-Dopa (50 μM) for 24 hours (n=3).

FIG. 10: shows the estimation of ATP level. Neurodegenerated cells were incubated with probucol (20 μM), SPQ (10 μM), HA-1 (10 μM) and L-Dopa (50 μM) for 24 hours (n=3).

FIG. 11: shows gene expression analysis by qRT-PCR for neuronal, dopaminergic and PD associated markers. (A) Synapsin I, (B) Nurr1, (C) DAT, (D) TH and (E) α-synuclein. (n=3).

FIG. 12: shows the effect of probucol, SPQ, HA-1 and L-Dopa on (A) intracellular reactive oxidative stress and (B) nitric oxide.

FIG. 13: shows the effect of probucol, SPQ, HA-1 and L-Dopa on glutathione activity.

FIG. 14: shows rotarod latency (average) in seconds at 12 weeks after sham procedure, sub-concussion (“SC”) with administration of AlN93M, sub-concussion with administration of probucol, sub-concussion with administration of AGE, and sub-concussion with administration of HA-1.

FIG. 15: shows the number of slips (Y axis) in a beam walk test on a 2 cm beam at 6 weeks and 12 weeks after sham procedure, sub-concussion with administration of AlN93M, sub-concussion with administration of probucol, sub-concussion with administration of AGE, and sub-concussion with administration of HA-1.

FIG. 16: shows NIT-1 cell viability at glucose 5.5 mM: under euglycemic conditions, none of the treatment groups exerted any effects and this was expected as under physiological conditions NIT-1 cells are already at optimal functionality.

FIG. 17: shows NIT-1 cell viability at glucose 25.5 mM: at hyperglycemic states, NIT-1 cells are under stress due to glucose-mediated toxicity, mitochondrial dysfunction and oxidative stress.

FIG. 18: shows the results of the Passive Avoidance Test at 14 weeks for negative control (non-diabetic mice), positive control (diabetic db/db mice), probucol, HA-1 and ApocIII.

FIG. 19: shows the results of the Novel Object Recognition Test at 14 weeks for negative control (non-diabetic mice), positive control (diabetic db/db mice), probucol, HA-1 and ApocIII.

FIG. 20: shows the effects of HA-1 on repeated subconcussion in rats as measured by rotarod and beamwalk tests: (A) shows rotarod latency (average) in seconds at 12 weeks after sham procedure, sub-concussion, sub-concussion with administration of probucol, and sub-concussion with administration of HA-1; and (B) shows the number of slips (Y axis) in a beam walk test on a 2 cm beam at 12 weeks after sham procedure, sub-concussion, sub-concussion with administration of probucol, and sub-concussion with administration of HA-1 (n=12).

FIG. 21: shows the increase in IgG extravasation in: (A) hippocampal formation (HPF), and (B) cortex (CTX) regions of the brain after 12-week repeated sub-concussion.

FIG. 22: shows the change in the expression of 8dOHG, a marker of oxidative stress, in: (A) hippocampal formation (HPF), and (B) cortex (CTX) regions of the brain after 12-week repeated sub-concussion.

FIG. 23: shows levels of: plasma glucose levels (A and B), plasma triglyceride levels (C and D), plasma insulin levels (E and F), and plasma cholesterol levels (G and H), measured in diabetic db/db mice at 14 weeks of age and at 28 weeks of age (n=12).

FIG. 24: shows levels of apoB (a marker of fat carrying particles) in the small intestine of diabetic db/db mice at 14 and 28 weeks of age (A and B), and levels of small intestinal Aβ of diabetic db/db mice at 14 and 28 weeks of age (C and D).

FIG. 25: shows levels of plasma Aβ40 in diabetic db/db mice at 14 and 28 weeks of age (A and B), levels of plasma Aβ42 in diabetic db/db mice at 14 and 28 weeks of age (C and D), plasma concentrations of Aβ oligomers in diabetic db/db mice at 14 and 28 weeks of age (E and F), and the Aβ42/40 ratio in diabetic db/db mice at 14 and 28 weeks of age (G and H) (n=12).

FIG. 26: shows the results of a passive avoidance latency test in diabetic db/db mice at 14 and 28 weeks of age (A and B) (n=12).

FIG. 27: shows levels of IgG extravasation in the hippocampal formation (HPF) in diabetic db/db mice at 14 and 28 weeks of age (A and B), and levels of IgG extravasation in the cortex (CTX) in diabetic db/db mice at 14 and 28 weeks of age (C and D) (n=12).

FIG. 28: shows levels of expression of 8dOHG in the HPF and CTX (A and B), levels of expression of GFAP in the HPF and CTX (C and D), and levels of expression of Iba-1 in the HPF and CTX (E and F) (n=12).

FIG. 29: shows the oxygen consumption rate (OCR) of OC-1 cells after 24 hours exposure to cisplatin.

FIG. 30: shows OCR data of OC-1 cells after 48 hours exposure to cisplatin followed by treatment with HA-1 and HA-2.

FIG. 31: shows the caspase assay results, with the analogues HA-1 and HA-2.

FIG. 32: shows cellular morphology and viability following cobalt chloride assays.

### DETAILED DESCRIPTION OF EMBODIMENTS

Preferred embodiments of the present invention are described below by way of example only.

### 1. Definitions

Unless otherwise herein defined, the following terms will be understood to have the general meanings which follow. The terms referred to below have the general meanings which follow when the term is used alone and when the term is used in combination with other terms, unless otherwise indicated. Hence, for example, the definition of “alkyl” applies to “alkyl” as well as the “alkyl” portions of “haloalkyl”, “arylalkyl”, etc.

The term “amphiphile” or “amphiphilic compound” as used herein is any compound having a hydrophilic/lipophilic balance (HLB) value between about 2 and about 20, inclusive. Preferred amphiphilic compounds have an HLB value between about 6 and about 16.

The term “alkyl” refers to a straight chain or branched chain saturated hydrocarbyl group. Unless indicated otherwise, preferred are C₁₋₆ alkyl and C₁₋₄ alkyl groups. The term “C_{x-y} alkyl”, where x and y are integers, refers to an alkyl group having x to y carbon atoms. For example, the term “C₁₋₆ alkyl” refers to an alkyl group having 1 to 6 carbon atoms. Examples of C₁₋₆ alkyl include methyl (Me), ethyl (Et), propyl (Pr), isopropyl (i-Pr), butyl (Bu), isobutyl (i-Bu), sec-butyl (s-Bu), tert-butyl (t-Bu), pentyl, neopentyl, hexyl and the like. Unless the context requires otherwise, the term “alkyl” also encompasses alkyl groups containing one less hydrogen atom such that the group is attached via two positions, i.e. divalent.

The term “alkenyl” refers to a straight chain or branched chain hydrocarbyl group having at least one double bond of either E- or Z-stereochemistry where applicable. Unless indicated otherwise, preferred are C₂₋₆ alkenyl and C₂₋₃ alkenyl groups. The term “C_{x-y} alkenyl”, where x and y are integers, refers to an alkenyl group having x to y carbon atoms. For example, the term “C₂₋₆ alkenyl” refers to an alkenyl group having 2 to 6 carbon atoms. Examples of C₂₋₆ alkenyl include vinyl, 1-propenyl, 1- and 2-butenyl and 2-methyl-2-propenyl. Unless the context requires otherwise, the term “alkenyl” also encompasses alkenyl groups containing one less hydrogen atom such that the group is attached via two positions, i.e. divalent.

The term “alkynyl” refers to a straight chain or branched chain hydrocarbyl group having at least one triple bond. Unless indicated otherwise, preferred are C₂₋₆ alkynyl and C₂₋₃ alkynyl groups. The term “C_{x-y} alkynyl”, where x and y are integers, refers to an alkynyl group having x to y carbon atoms. For example, the term “C₂₋₆ alkynyl” refers to an alkynyl group having 2 to 6 carbon atoms. Examples of C₂₋₆ alkynyl include ethynyl, 1-propynyl, 1- and 2-butynyl, 2-pentynyl, 3-pentynyl, 4-pentynyl, 2-hexynyl, 3-hexynyl, 4-hexynyl and 5-hexynyl and the like. Unless the context indicates otherwise, the term “alkynyl” also encompasses alkynyl groups containing one less hydrogen atom such that the group is attached via two positions, i.e. divalent.

The term “cycloalkyl” refers to a non-aromatic cyclic hydrocarbyl group having 3 or more carbon atoms in the ring. The term “C₃₋₃₀ cycloalkyl” refers to a non-aromatic cyclic hydrocarbyl group having from 3 to 30 carbon atoms. Such groups include cyclopropyl, cyclobutyl, cyclopentyl, cyclohexyl, cycloheptyl and cyclooctyl. The term “C₃₋₃₀ cycloalkyl” encompasses groups where the cyclic hydrocarbyl group is saturated such as cyclohexyl or unsaturated such as cyclohexenyl. In some embodiments, the C₃₋₃₀ cycloalkyl is C₃₋₈ cycloalkyl. C₃₋₆ cycloalkyl such as cyclopropyl, cyclobutyl, cyclopentyl and cyclohexyl are preferred.

The term “acyloxy” refers to the group —OC(═O)R^{m} wherein R^{m} is a C₁₋₃₀ alkyl group.

The terms “hydroxy” and “hydroxyl” refer to the group —OH.

The term “oxo” refers to the group ═O.

The term “alkoxy” refers to an alkyl group as defined above covalently bound via an O linkage, such as methoxy, ethoxy, propoxy, isopropoxy, butoxy, tert-butoxy and pentoxy. Unless indicated otherwise, preferred are C₁₋₆ alkoxy, C₁₋₄ alkoxy and C₁₋₃ alkoxy groups.

The term “aryl” refers to a carbocyclic (non-heterocyclic) aromatic ring or mono-, bi- or tri-cyclic ring system. The aromatic ring or ring system is generally composed of 6 to 10 carbon atoms. Examples of aryl groups include but are not limited to phenyl, biphenyl, naphthyl and tetrahydronaphthyl. 6-Membered aryls such as phenyl are preferred.

The term “arylalkyl” or “aralkyl” refers to an arylC₁₋₆alkyl—such as benzyl.

The term “heteroaryl” is used herein to denote a heterocyclic group having aromatic character and embraces aromatic monocyclic ring systems and polycyclic (e.g. bicyclic) ring systems containing one or more aromatic rings. The term aromatic heterocyclyl also encompasses pseudoaromatic heterocyclyls. The term “pseudoaromatic” refers to a ring system which is not strictly aromatic, but which is stabilized by means of delocalization of electrons and behaves in a similar manner to aromatic rings. The term aromatic heterocyclyl therefore covers polycyclic ring systems in which all of the fused rings are aromatic as well as ring systems where one or more rings are non-aromatic, provided that at least one ring is aromatic. In polycyclic systems containing both aromatic and non-aromatic rings fused together, the group may be attached to another moiety by the aromatic ring or by a non-aromatic ring.

Examples of heteroaryl groups are monocyclic and bicyclic groups containing from five to ten ring members. The heteroaryl group can be, for example, a five membered or six membered monocyclic ring or a bicyclic structure formed from fused five and six membered rings or two fused six membered rings or two fused five membered rings. Each ring may contain up to four heteroatoms selected from nitrogen, sulphur and oxygen. The heteroaryl group can contain up to 4 heteroatoms, more typically up to 3 heteroatoms, more usually up to 2 heteroatoms. In one embodiment, the heteroaryl group contains at least one ring nitrogen atom. The nitrogen atoms in the heteroaryl group can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of heteroaryl groups containing an aromatic ring and a non-aromatic ring include tetrahydroisoquinoline, tetrahydroquinoline, dihydrobenzothiophene, dihydrobenzofuran, 2,3-dihydro-benzo[1,4]dioxine, benzo[1,3]dioxole, 4,5,6,7-tetrahydrobenzofuran, indoline and isoindoline groups.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

The term “stereoisomers” refers to compounds which have identical chemical constitution with atoms connected in the same order, but differ with regard to the spatial arrangement of the atoms.

Throughout this specification, including the claims, the word “include”, or variations thereof such as “includes” or “including”, will be understood to imply the inclusion of a stated element, integer or step, or group of elements, integers or steps, but not the exclusion of any other element, integer or step, or group of elements, integers or steps.

Throughout this specification, including the claims, except where the context requires otherwise due to express language or necessary implication, the word “comprise” or variations such as “comprises” or “comprising” is used in an inclusive sense, i.e. to specify the presence of a stated element, integer or step, or group of elements, integers or steps, but not to preclude the presence or addition of a further element, integer or step, or a further group of elements, integers or steps, in various embodiments of the invention.

The term “pharmaceutically acceptable carrier” as used herein refers to a carrier or excipient or diluent that is suitable for use with humans and/or animals without undue adverse side effects (such as toxicity, irritation, and allergic response) commensurate with a reasonable benefit/risk ratio. It may be a pharmaceutically acceptable solvent, suspending agent or vehicle, for delivering active ingredients to the subject.

The term “controlled release” as used herein refers to the control of the rate and/or quantity of active ingredients delivered according to the pharmaceutical compositions described herein. The controlled release kinetics may be prolonged or sustained release, fast or immediate release, delayed release or pulsatile drug delivery system.

The terms “individual”, “subject” and “patient” are used herein interchangeably. In certain embodiments, the subject is a mammal. Mammals include, but are not limited to, primates (including human and non-human primates). In a preferred embodiment, the subject is a human.

The term “neurological disorder” as used herein is defined as any disorder that affects the brain as well as the nerves found throughout the human body and the spinal cord. Neurological disorders include neurodegenerative conditions such as Alzheimer's disease, dementia caused by Alzheimer's disease, Parkinson's disease, amyotrophic lateral sclerosis (motor neurone disease), multiple sclerosis, and brain injury, and can result in cognitive decline (such as memory loss).

The term “disorder caused by stress-induced cellular damage in the middle or inner ear” as used herein is defined as any disorder which is caused by stress-induced cellular damage in the middle or inner ear. The cellular damage in the middle or inner ear may be induced by any form of insult that can increase cellular stress in the middle or inner ear via molecular disturbances, including for example, by a physical or acoustic trauma, a bacterial, viral or other type of infection, or chemical ototoxic insult, resulting in high levels of free-radicals, oxidative stress, pro-inflammatory cytokines, affecting normal cellular function in the middle inner ear, causing inflamed or damaged specialized middle or inner ear epithelia such as stria vascularis, spiral ligament, organ of Corti, endolymphatic sac, crista amplularis, otoliths, spiral ganglion, auditory nerves, along with neuronal signalling loss and general apoptosis throughout the middle or inner ear. Changes in the middle ear can affect the inner ear. For example, otitis media, a primarily middle ear problem, can result in sensorineural hearing loss and imbalance, which are both inner ear problems.

The term “hearing disorder” as used herein is defined as any disorder caused by molecular disturbances affecting the inner or middle ear and their relevant neural connections to the brain.

The term “hearing impairment” as used herein is defined as any impairment of hearing caused by molecular disturbances affecting the inner or middle ear and their relevant neural connections to the brain.

The term “hearing loss” as used herein is defined as a diminished ability to perceive sounds relative to normative levels. This may be caused either by a conductive hearing loss, sensorineural hearing loss, or a combination of both. Hearing loss includes presbycusis (age-related hearing loss).

The term “conductive hearing loss” as used herein is one where sound pressure transmission from the external to inner ear is attenuated, for example, due to an excessive build-up of earwax, glue ear, an ear infection with inflammation and fluid build-up, a perforated or defective eardrum, a skin growth in the middle ear (cholesteatoma), or a malfunction of the ossicles (bones in the middle ear).

The term “sensorineural hearing loss” as used herein is caused by dysfunction of the sensory and/or neural cells of the cochlea, and/or dysfunction of the specialized epithelia in the inner ear such as stria vacularis and cochlear supporting cells, as well as their relevant neural connections from the ear to the brain.

The term “vestibular dysfunction” as used herein is any impairment of balance or vision stabilization caused by molecular disturbances affecting the inner ear.

The term “hair cell degeneration” or “hair cell loss” as used herein refers to a gradual loss of hair cell function and integrity and/or leading ultimately to hair cell death.

The term “hair cell death” as used herein refers to apoptosis of the hair cells in the middle or inner ear.

The terms “identification of hair cell damage” and “detection of hair cell damage” are used interchangeably herein and refer to a method by which the degree of hair cell damage in the middle or inner ear may be determined. Such methods are known in the art and comprise, for example, fluorescent imaging of the hair cells. An audiogram that demonstrates loss of hearing sensitivity at moderate to high frequencies is also indicative of hair cell damage. A decrease of hearing potential with no subsequent recovery is also diagnostic of hair cell damage.

The terms “chemically induced hearing loss” and “hearing loss induced by a chemical” as used herein refer to hearing loss which is induced and/or caused by chemical agents, such as solvents, gases, paints, heavy metals, and/or medicaments which are ototoxic.

An “effective amount” or “therapeutically effective amount” is an amount sufficient to effect a beneficial or desired therapeutic effect. This amount may be the same or different from a “prophylactically effective amount”, which is an amount necessary to prevent onset of the disorder or disorder symptoms. An effective amount may be administered in one or more administrations, applications or dosages. A therapeutically effective amount of a therapeutic compound (i.e., an effective dosage) depends on the therapeutic compounds selected.

The term “treating” as used herein refers to affecting a subject, tissue or cell to obtain a desired pharmacological and/or physiological effect and includes inhibiting the condition, i.e. arresting its development; or relieving or ameliorating the effects of the condition i.e., cause reversal or regression of the effects of the condition.

The term “preventing” as used herein refers to preventing a condition from occurring in a cell, tissue or subject that may be at risk of having the condition, but does not necessarily mean that condition will not eventually develop, or that a subject will not eventually develop a condition. Preventing includes delaying the onset of a condition in a cell, tissue or subject.

The singular terms “a”, “an”, and “the” include plural referents unless the context clearly indicates otherwise. Similarly, the word “or” is intended to include “and” unless the context clearly indicates otherwise. Although methods and materials similar or equivalent to those described herein may be used in the practice or testing of this disclosure, suitable methods and materials are described below.

The abbreviation, “e.g.” is derived from the Latin exempli gratia, and is used herein to indicate a non-limiting example. Thus, the abbreviation “e.g.” is synonymous with the term “for example”.

The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

The term “about” as used herein refers to +/−up to 20% of a given measurement. For example, the term “about” refers to a value that is within plus or minus 10% or within plus or minus 5% of the recited value.

The recitation of ranges of values herein is merely intended to serve as a shorthand method of referring individually to each separate value falling within the range. Unless otherwise indicated herein, each individual value is incorporated into the specification as if it were individually recited herein.

All methods described herein may be performed in any suitable order unless otherwise indicated herein or otherwise clearly contradicted by context.

### 2. Compounds of Formula (I)

The present invention broadly relates to compounds of Formula (I), including stereoisomers of the compounds of Formula (I) and pharmaceutically acceptable salts of the compounds of Formula (I). The compounds of Formula (I) are formed by conjugating probucol or succinobucol to a bile acid, or by conjugating probucol or succinobucol to a derivative of a bile acid. The conjugate of Formula (I) is also referred to herein as a “bile acid conjugate”. A reference herein to a “compound of Formula (I)” includes stereoisomers of Formula (I), and any mixture of stereoisomers of Formula (I).

Bile acids are steroids whose structure is related to cholane or cholestane. Bile acids may be termed “cholanoids” when it is convenient to have a name for this subclass of steroids. The term “bile acid” is a generic term for such molecules with a carboxyl group and does not denote any state of ionization. The numbering of the carbon atoms of the steroid ring and side chain is depicted below in the structure of the cholestane skeleton.

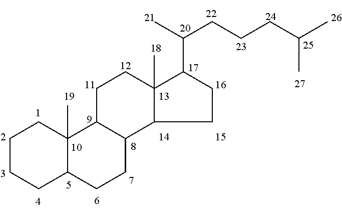 [SMILES: CC(C)CCCC(C)C1CCC2C3CCC4CCCCC4(C)C3CCC12C]

Bile acids are facial amphiphiles, i.e. they contain both hydrophobic and hydrophilic faces. The cholesterol-derived portion of a bile acid has one face that is hydrophobic (with methyl groups) and one that is hydrophilic (with the hydroxyl groups).

Examples of bile acids that may be used to prepare the compound of Formula (I) or a stereoisomer thereof, or a pharmaceutically acceptable salt thereof, include:

| Bile acid | Hydroxyl substituents |
|---|---|
| cholic acid | 3α, 7α, 12α |
| deoxycholic acid | 3α, 12α |
| chenodeoxycholic acid | 3α, 7α, |
| ursodeoxycholic acid | 3α, 7β |
| lithocholic acid | 3α |
| muricholic acid | |
| α-muricholic acid | 3α, 6β, 7α |
| β-muricholic acid | 3α, 6β, 7β |
| γ-muricholic acid | 3α, 6α, 7α |
| ω-muricholic acid | 3α, 6α, 7β |
| murideoxycholic acid | 3α, 6β |
| hyocholic acid (2 epimers) | |
| α-hyocholic acid | 3α, 6α, 7α |
| β-hyocholic acid | 3α, 6α, 7β |
| hyodeoxycholic acid | 3α, 6α |
| ursocholic acid | 3α, 7β, 12α |
| lagocholic acid | 3α, 7α, 12β |
| lagodeoxycholic acid | 3α, 12β |
| cricetocholic acid | 3α, 5β, 7α |
| vulpecholic acid | 1α, 3α, 7α |
| glycocholic acid | 3α, 7α, 12α |
| glycochenodeoxycholic acid | 3α, 7α |
| taurocholic acid | 3α, 7α, 12α |
| taurochenodeoxycholic acid | 3α, 7α |
| glycodeoxycholic acid | 3α, 12α |
| taurodeoxycholic acid | 3α, 12α |
| glycolithocholic acid | 3α |
| taurolithocholic acid | 3α |
| tauroursodeoxycholic acid | 3α, 7β |
| obeticholic acid | 3α, 7α |
| glycomuricholic acid | |
| α-glycomuricholic acid | 3α, 6β, 7α |
| β-glycomuricholic acid | 3α, 6β, 7β |
| γ-glycomuricholic acid | 3α, 6α, 7α |
| ω-glycomuricholic acid | 3α, 6α, 7β |
| tauromuricholic acid | |
| α-tauromuricholic acid | 3α, 6β, 7α |
| β-tauromuricholic acid | 3α, 6β, 7β |
| γ-tauromuricholic acid | 3α, 6α, 7α |
| ω-tauromuricholic acid | 3α, 6α, 7β |

Probucol (2,6-ditert-butyl-4-[2-(3,5-ditert-butyl-4-hydroxyphenyl)sulfanylpropan-2-ylsulfanyl]phenol) is a hydrophobic compound which has antioxidative properties, and this may be due to its phenolic structure. Probucol is a potent oxygen radical scavenger that can serve as a powerful anti-inflammatory agent to suppress oxidant induced tissue injury, in addition to being a cholesterol reducing and anti-atherogenic drug. Because glutathione peroxidase (GPx) plays a crucial role in preventing oxidative stress, the pharmacological use of its mimetics has been proposed as a strategy to treat oxidative stress-related pathological condition. In some studies, the protective effects of probucol were paralleled by significant increases in GPx activity.

The inventors have found that conjugating probucol to a bile acid results in a conjugate (referred to herein as a “bile acid conjugate”) having useful and beneficial properties and effects, such as neuroprotective effects, anti-inflammatory activity, antioxidative properties, and epithelial cell protective effects.

Advantageously, the bile acid conjugate may be used in quantities that are effective to produce the desired therapeutic effect, but with reduced toxicity, and with reduced or no adverse side effects usually associated with probucol and succinobucol. The bile acid conjugate is expected to provide greater tissue penetration, and enhanced pharmacodynamic response with improved pharmacokinetic profile due to its stability, lipophilicity and tissue uptake. Thus, more predictable dose-response effects would be achieved and this in turn would minimise side-effects.

Without wishing to be bound by theory, the inventors believe that the conjugate, being a compound of Formula (I), may act as neuroprotective agent, an anti-inflammatory agent, an antioxidant, and/or an epithelial cell protective agent, and provide cell protective effects.

In one aspect, the present invention provides a compound of Formula (I):

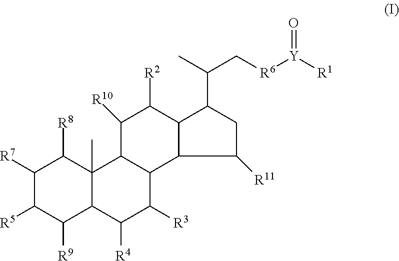

wherein:
R¹ is:

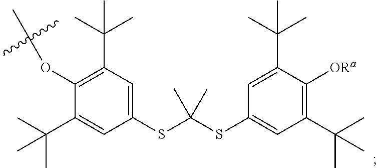

each R², R³, R⁴, R⁵, R⁷, R⁸, R⁹, R¹⁰ and R¹¹ is independently selected from H, substituted or unsubstituted C₁₋₃₀ acyloxy, substituted or unsubstituted benzoyloxy, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, substituted or unsubstituted aryl, substituted or unsubstituted arylalkyl, substituted or unsubstituted heteroaryl, an amino acid moiety, or GL, wherein GL is independently selected from OL, SL, PL₂, CL₃, or NL₂; wherein when R², R³, R⁴, R⁵, R⁷, R⁸, R⁹, R¹⁰ or R¹¹ is substituted, the substituent is independently selected from OH, F, SH, ═O, ═S, Cl, Br, SC₁₋₆ alkyl, C₁₋₆ alkyl or C₁₋₆ alkoxy;
each L is independently selected from H, a metal ion, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, a substituted or unsubstituted benzyl radical, —CH₂CO₂H, or —(CH₂)₂SO₃H; wherein when L is substituted, the substituent is independently selected from OH, SH, ═O, ═S, F, Cl, Br, SC₁₋₆ alkyl, C₁₋₆ alkyl or C₁₋₆ alkoxy;
R⁶ is —(CH₂)ₙ— wherein n is 0 to 12, —CH₂C(═O)NHCH₂—, or —CH₂C(═O)NHCH₂CH₂—;
Y is —C— or —S(═O)—; and
R^{a} is —H or —C(═O)CH₂CH₂COOH;
or a stereoisomer thereof, or a pharmaceutically acceptable salt thereof.

In some embodiments, R^{a} is —H. Thus, in some embodiments, R¹ is:

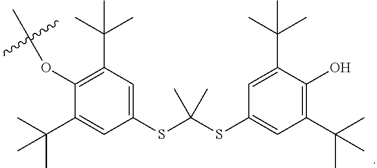 [SMILES: *Oc1c(C(C)(C)C)cc(SC(C)(C)Sc2cc(C(C)(C)C)c(O)c(C(C)(C)C)c2)cc1C(C)(C)C]

In some embodiments, R^{a} is —C(═O)CH₂CH₂COOH. Thus, in some embodiments, R¹ is:

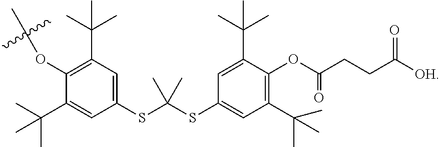 [SMILES: *Oc1c(C(C)(C)C)cc(SC(C)(C)Sc2cc(C(C)(C)C)c(OC(=O)CCC(=O)O)c(C(C)(C)C)c2)cc1C(C)(C)C]

In some embodiments, each R², R³, R⁴, R⁵, R⁷, R⁸, R⁹, R¹⁰ and R¹¹ is independently selected from H; substituted or unsubstituted C₁₋₃₀ acyloxy (e.g. substituted or unsubstituted C₁₋₆ acyloxy or substituted or unsubstituted C₁₋₄ acyloxy); substituted or unsubstituted benzoyloxy; substituted or unsubstituted C₁₋₁₂ alkyl (e.g. substituted or unsubstituted C₁₋₆ alkyl or substituted or unsubstituted C₁₋₄ alkyl); substituted or unsubstituted C₂₋₁₂ alkenyl (e.g. substituted or unsubstituted C₂₋₆ alkenyl or substituted or unsubstituted C₂₋₄ alkenyl); substituted or unsubstituted C₂₋₁₂ alkynyl (e.g. substituted or unsubstituted C₂₋₆ alkynyl or substituted or unsubstituted C₂₋₄ alkynyl); substituted or unsubstituted C₃₋₈ cycloalkyl; substituted or unsubstituted C aryl (e.g. substituted or unsubstituted phenyl); substituted or unsubstituted heteroaryl (e.g. substituted or unsubstituted pyridyl), or an amino acid moiety. In some embodiments, when R², R³, R⁴, R⁵, R⁷, R⁸, R⁹, R¹⁰ or R¹¹ is substituted, the substituent is independently selected from F, Cl, OH, SH, Br, SC₁₋₄ alkyl, C₁₋₄ alkyl or C₁₋₄ alkoxy. In some embodiments, when R², R³, R⁴, R⁵, R⁷, R⁸, R⁹, R¹⁰ or R¹¹ is substituted, it is substituted with 1, 2, 3 or 4 substituents independently selected from F, Cl, Br, OH, SH, ═O, ═S, SC₁₋₄ alkyl, C₁₋₄ alkyl or C₁₋₄ alkoxy.

In some embodiments, GL is selected from OL, SL, PL₂, CL₃, or NL₂, wherein each L is independently selected from H, a metal ion, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, a substituted or unsubstituted benzyl radical, —CH₂CO₂H, or —(CH₂)₂SO₃H; wherein when L is substituted, the substituent is independently selected from OH, SH, ═O, ═S, F, Cl, Br, SC₁₋₆ alkyl, C₁₋₆ alkyl or C₁₋₆ alkoxy.

In some embodiments, L is H; substituted or unsubstituted C₁₋₁₂ alkyl (e.g. substituted or unsubstituted C₁₋₆ alkyl or substituted or unsubstituted C₁₋₄ alkyl); substituted or unsubstituted C₂₋₁₂ alkenyl (e.g. substituted or unsubstituted C₂₋₆ alkenyl or substituted or unsubstituted C₂₋₄ alkenyl); substituted or unsubstituted C₂₋₁₂ alkynyl (e.g. substituted or unsubstituted C₂₋₆ alkynyl or substituted or unsubstituted C₂₋₄ alkynyl); substituted or unsubstituted C₃₋₈ cycloalkyl; a substituted or unsubstituted benzyl radical; —CH₂CO₂H; or —(CH₂)₂SO₃H. In some embodiments, when L is substituted, the substituent is independently selected from F, Cl, OH, SH, ═O, ═S, Br, SC₁₋₄ alkyl, C₁₋₄ alkyl or C₁₋₄ alkoxy. In some embodiments, when L is substituted, it is substituted with 1, 2, 3 or 4 substituents independently selected from F, Cl, OH, SH, ═O, ═S, Br, SC₁₋₄ alkyl, C₁₋₄ alkyl or C₁₋₄ alkoxy.

In some embodiments, GL is OH.

In some embodiments, L is a metal ion selected from, for example, metal(I) ions (e.g. Na⁺, K⁺, and Li⁺), and metal(II) ions (e.g. cadmium(II) ion, iron(II) ion, lead(II) ion, zinc(II) ion, copper(II) ion, magnesium(II) ion, and manganese(II) ion). Thus in some embodiments, a metal cation (e.g. Na⁺, K⁺, Li⁺, Cd²⁺, Fe²⁺, Pb²⁺, Zn²⁺, Cu²⁺, Mg²⁺, or Mn²⁺) is bonded to an organic moiety.

In some embodiments, R², R³, R⁴, R⁵, R⁷, R⁸, R⁹, R¹⁰ or R¹¹ may be an amino acid moiety. The amino acid moiety may be formed from any amino acid, such as, for example, any of the available main types of amino acids. For example, the amino acid may be selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and taurine. In some embodiments, the amino acid is glycine, taurine or alanine. In some embodiments, the amino acid is glycine. In some embodiments, the amino acid is taurine.

In some embodiments, each R², R³, R⁴, R⁵, R⁷, R⁸, R⁹, R¹⁰ and R¹¹ is independently selected from H, —OH and —OC(═O)R^{f}, wherein R^{f} is C₁₋₆ alkyl.

In some embodiments, R⁶ is —(CH₂)ₙ— wherein n is 0, 1, 2, 3, 4, 5, 6, 7, 8, 9, 10, 11 or 12. In some embodiments, n is 0. In some embodiments, n is 1, 2, 3, 4, 5, 6, 7, 8, 9, 10, 11 or 12. In some embodiments, n is 1, 2, 3, 4 or 5. Preferably, n is 1. In some embodiments, R⁶ is —CH₂—.

In some embodiments, R⁶ is —CH₂C(═O)NHCH₂—. In some embodiments, R⁶ is —CH₂C(═O)NHCH₂CH₂—.

In some embodiments, Y is —C—. In some embodiments, Y is —S(═O)—.

In some embodiments, the compound of the Formula (I) has two or more hydroxy (—OH) groups attached to two different rings of Formula (I), e.g. two different rings of a carboxylated steroidal compound of Formula (I).

Each R², R³, R⁴, R⁵, R⁷, R⁸, R⁹, R¹⁰ and R¹¹, the —CH₃ groups at C10 and C13, the hydrogen atoms at C5, C8, C9 and C14, and the group on C17 may be attached in the α or β configuration on the respective ring. For example, the R⁵ group may be positioned 3α or 3β.

In one embodiment, the compound of Formula (I) is a compound of Formula (Ia):

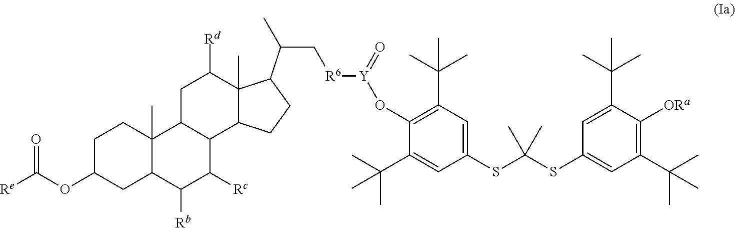

wherein:
R^{a} is —H or —C(═O)CH₂CH₂COOH;
R^{b} is selected from —H, —OH and —OC(═O)R^{f}, wherein R^{f} is C₁₋₆ alkyl;
R^{c} is selected from —H, —OH and —OC(═O)R^{f}, wherein R^{f} is C₁₋₆ alkyl;
R^{d} is selected from —H, —OH and —OC(═O)R^{f}, wherein R^{f} is C₁₋₆ alkyl;
R^{e} is selected from —C₁₋₆ alkyl;
R⁶ is —CH₂—, —CH₂C(═O)NHCH₂—, or —CH₂C(═O)NHCH₂CH₂—; and
Y is C or S(═O);
or a stereoisomer thereof, or a pharmaceutically acceptable salt thereof.

In some embodiments of Formula (Ia), R^{a} is —H. In some embodiments of Formula (Ia), R^{a} is —C(═O)CH₂CH₂COOH.

Thus, in some embodiments, Formula (Ia) is:

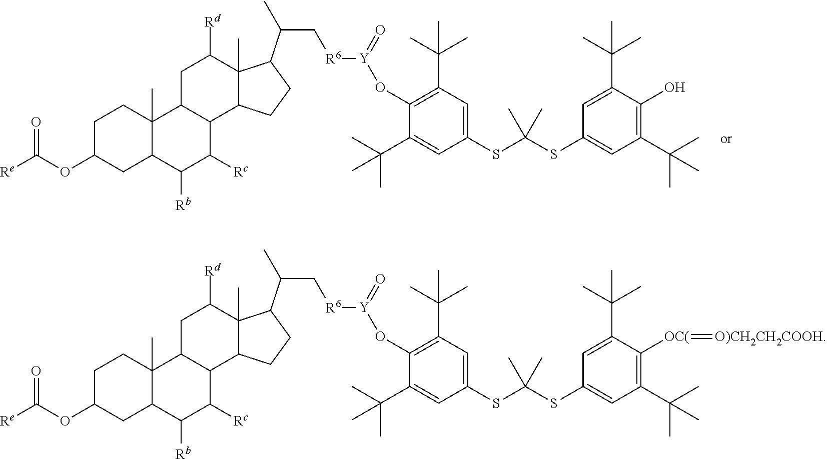

In some embodiments of Formula (Ia), each R^{b}, R^{c} and R^{d} is independently selected from —H, —OH and —OC(═O)R^{f}, wherein R^{f} is C₁₋₆ alkyl. In some embodiments, R^{f} is —C₁₋₆ alkyl, —C₁₋₅ alkyl, —C₁₋₄ alkyl, or —C₁₋₃ alkyl. In some embodiments, R^{f} is methyl (Me), ethyl (Et), propyl (Pr), isopropyl (i-Pr), butyl (Bu), isobutyl (i-Bu), sec-butyl (s-Bu), tert-butyl (t-Bu), pentyl, neopentyl, or hexyl.

In some embodiments of Formula (Ia), R^{e} is selected from —C₁₋₆ alkyl, —C₁₋₅ alkyl, —C₁₋₄ alkyl, or —C₁₋₃ alkyl. In some embodiments, R^{e} is methyl (Me), ethyl (Et), propyl (Pr), isopropyl (i-Pr), butyl (Bu), isobutyl (i-Bu), sec-butyl (s-Bu), tert-butyl (t-Bu), pentyl, neopentyl, or hexyl.

In some embodiments of Formula (Ia), R⁶ is —CH₂—. In some embodiments of Formula (Ia), R⁶ is —CH₂C(═O)NHCH₂—. In some embodiments of Formula (Ia), R⁶ is —CH₂C(═O)NHCH₂CH₂—.

In some embodiments of Formula (Ia), Y is C. In some embodiments of Formula (Ia), Y is S(═O).

In some embodiments, the compound of Formula (Ia) is 2,6-di-tert-butyl-4-((2-((3,5-di-tert-butyl-4-hydroxyphenyl)thio)propan-2-yl)thio)phenyl (4R)-4-[(3R,5R,8R,9S,10S,13R,14S,17R)-3-acetoxy-10,13-dimethyl-2,3,4,5,6,7,8,9,11,12,14,15,16,17-tetradecahydro-1H-cyclopenta[a]phenanthren-17-yl]pentanoate (referred to herein as “HA-1”):

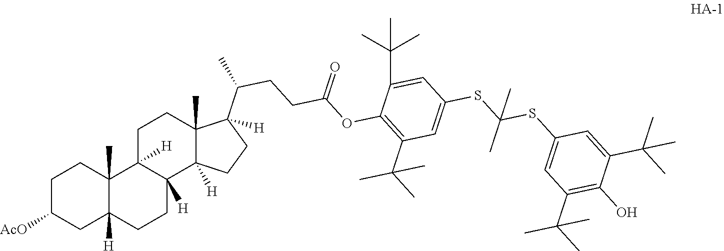

In some embodiments, the compound of Formula (Ia) is 2,6-di-tert-butyl-4-((2-((3,5-di-tert-butyl-4-hydroxyphenyl)thio)propan-2-yl)thio)phenyl (4R)-4-[(3R,5R,7R,8R,9S,10S,13R,14S,17R)-3,7-diacetoxy-10,13-dimethyl-2,3,4,5,6,7,8,9,11,12,14,15,16,17-tetradecahydro-1H-cyclopenta[a]phenanthren-17-yl]pentanoate (referred to herein as “HA-2”):

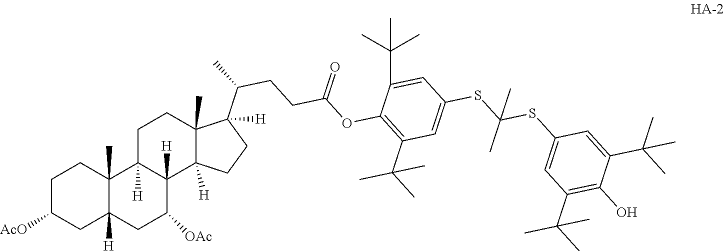

HA-1 is probucol conjugated with lithocholic acid. HA-2 is probucol conjugated with chenodeoxycholic acid.

The salts of the compounds of the Formula (I) are pharmaceutically acceptable. When compounds of the Formula (I) contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, β-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the Formula (I) contain both basic and acidic functionalities that allow the compounds to be converted into either base or acid addition salts. Other pharmaceutically acceptable carriers known to those of skill in the art are suitable for the present invention.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The compounds of the Formula (I) may be synthesised by methods known in the art of organic synthesis. Methods for optimizing reaction conditions, if necessary minimising competing by-products, are known in the art. Reaction optimisation and scale-up may advantageously utilize high-speed parallel synthesis equipment and computer-controlled microreactors (e.g. Design And Optimization in Organic Synthesis, 2^{nd} Edition, Carlson R, Ed, 2005; Elsevier Science Ltd.; Jähnisch, K et al, Angew. Chem. Int. Ed. Engl. 2004 43: 406; and references therein). Additional reaction schemes and protocols may be determined by the skilled artesian by use of commercially available structure-searchable database software, for instance, SciFinder® (CAS division of the American Chemical Society) and CrossFire Beilstein® (Elsevier MDL), or by appropriate keyword searching using an internet search engine such as Google® or keyword databases such as the US Patent and Trademark Office text database. The invention includes the intermediate compounds used in making the compounds of the formulae herein as well as methods of making such compounds and intermediates, including without limitation those as specifically described in the examples herein.

The compounds of the Formula (I) may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present invention. The compounds herein may also be represented in multiple tautomeric forms, in such instances, the invention expressly includes all tautomeric forms of the compounds described herein, even though only a single tautomeric form may be represented. All such isomeric forms of such compounds herein are expressly included in the present invention. Also embodied are extracts and fractions comprising a compound of Formula (I) or a stereoisomer thereof. The term “isomers” is intended to include diastereoisomers, enantiomers, regioisomers, rotational isomers, tautomers, and the like. For compounds which contain one or more stereogenic centres, e.g., chiral compounds, the methods described herein may be carried out with an enantiomerically enriched compound, a racemate, or a mixture of diastereomers.

Preferred enantiomerically enriched compounds have an enantiomeric excess of 50% or more, more preferably the compound has an enantiomeric excess of 60%, 70%, 80%, 90%, 95%, 98%, or 99% or more. In preferred embodiments, only one enantiomer or diastereomer of a chiral compound of Formula (I) or a stereoisomer thereof, or a pharmaceutically acceptable salt thereof, is administered to cells or a subject.

### 3. Preparation Methods for the Compounds of Formula (I)

The compounds of Formula (I) may be synthesised by methods known in the art. Various synthetic schemes are described below and in the Examples. The Examples describe the preparation of various specific compounds of Formula (I). A person skilled in the art would be able to modify the synthetic schemes described below, and in the Examples, to prepare other compounds of Formula (I) and salts thereof.

A typical general procedure for preparing compounds of Formula (I) from a bile acid and probucol is described below.

Examples of bile acids that may be used in preparation of the compounds of Formula (I) include: deoxycholic acid, cholic acid, taurocholic acid, glycocholic acid, glycodeoxycholic acid, taurodeoxycholic acid, ursodeoxycholic acid, taurochenodeoxycholic acid, lithocholic acid, glycolithocholic acid, chenodeoxycholic acid, taurolithocholic acid, tauroursodeoxycholic acid, obeticholic acid, any muricholic acid (e.g. α-muricholic acid, β-muricholic acid, γ-muricholic acid, ω-muricholic acid), glycomuricholic acid, tauromuricholic acid, or glycochenodeoxycholic acid, or a salt thereof.

For example, the starting bile acid may be a compound of the following formula (B1):

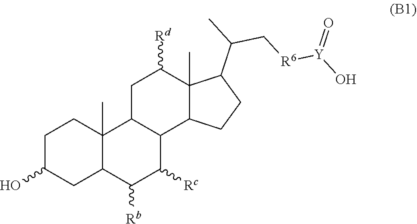

| | Bile acid | R^{b} | R^{c} | R^{d} | R⁶ | Y |
|---|---|---|---|---|---|---|
| (a) | Cholic acid | H | OH | OH | —CH₂— | C |
| (b) | Lithocholic acid | H | H | H | —CH₂— | C |
| (c) | Ursodeoxycholic acid | H | OH | H | —CH₂— | C |
| (d) | Chenodeoxycholic acid | H | OH | H | —CH₂— | C |
| (e) | Deoxycholic acid | H | H | OH | —CH₂— | C |
| (f) | Muricholic acid | OH | OH | H | —CH₂— | C |
| (g) | Glycocholic acid | H | OH | OH | —CH₂C(═O)NHCH₂— | C |
| (h) | Glycochenodeoxycholic acid | H | OH | H | —CH₂C(═O)NHCH₂— | C |
| (i) | Taurocholic acid | H | OH | OH | —CH₂C(═O)NHCH₂CH₂— | S═O |
| (j) | Taurochenodeoxycholic acid | H | OH | H | —CH₂C(═O)NHCH₂CH₂— | S═O |

The compound of formula (B1) may be dissolved in pyridine, and then acetic anhydride may be added to form the O-acyl protected bile acid of formula (B2):

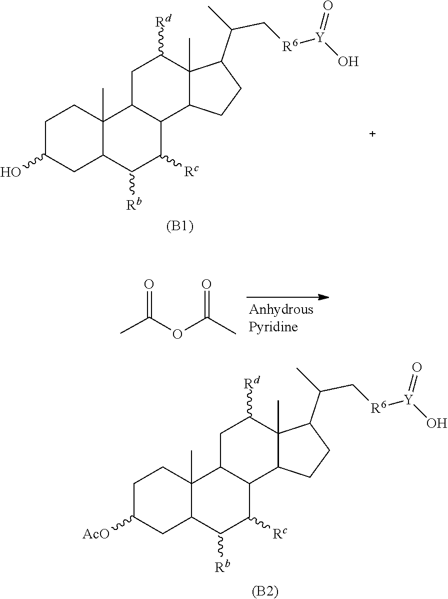

wherein, in formula (B2), R^{b}, R^{c}, R^{d}, R⁶, and Y have following meanings:

| | R^{b} | R^{c} | R^{d} | R⁶ | Y |
|---|---|---|---|---|---|
| (a) | H | OAc | OAc | —CH₂— | C |
| (b) | H | H | H | —CH₂— | C |
| (c) | H | OAc | H | —CH₂— | C |
| (d) | H | OAc | H | —CH₂— | C |
| (e) | H | H | OAc | —CH₂— | C |
| (f) | OAc | OAc | H | —CH₂— | C |
| (g) | H | OAc | OAc | —CH₂C(═O)NHCH₂— | C |
| (h) | H | OAc | H | —CH₂C(═O)NHCH₂— | C |
| (i) | H | OAc | OAc | —CH₂C(═O)NHCH₂CH₂— | S═O |
| (j) | H | OAc | H | —CH₂C(═O)NHCH₂CH₂— | S═O |

The protected bile acid of formula (B2) is then reacted with, e.g. thionyl chloride or oxalyl chloride, to give the O-acyl bile acid chloride of formula (B3):

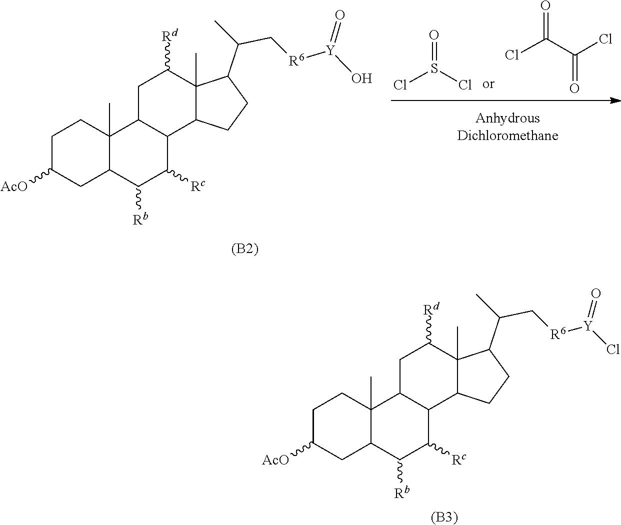

Separately, the potassium salt of probucol may be prepared by adding probucol to a mixture of potassium tert-butoxide stirred in anhydrous tetrahydrofuran:

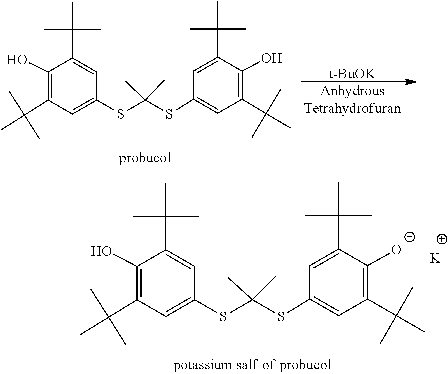

The O-acetyl bile acid chloride of formula (B3) is then reacted with the potassium salt of probucol to form the bile acid conjugate of formula (B4):

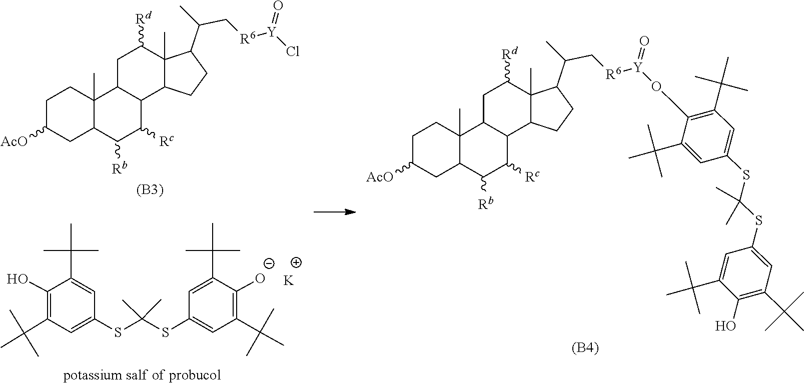

In the above general procedure, succinobucol may be used in place of probucol to produce a bile acid conjugate with succinobucol.

Any bile acid or bile salt may be used in the preparation of the compound of Formula (I), such as those referred to above. For example, the bile acid or bile salt may be a primary bile acid, or a secondary bile acid or any bile acid with an amino acid moiety attached. Various bile acids and bile salts are commercially available. For example, the bile acid or bile salt may be selected from deoxycholic acid (DCA), ursodeoxycholic acid (UDCA; also known as ursodiol), cholic acid, taurocholic acid, glycocholic acid, glycodeoxycholic acid, glycochenodeoxycholic acid, taurodeoxycholic acid, taurochenodeoxycholic acid, lithocholic acid, glycolithocholic acid, chenodeoxycholic acid, taurolithocholic acid, tauroursodeoxycholic acid, obeticholic acid, all muricholic acids (α-muricholic acid, β-muricholic acid, γ-muricholic acid and ω-muricholic acid), glycomuricholic acid, tauromuricholic acid, or a salt, derivative or metabolite thereof. In some embodiments, the bile acid is deoxycholic acid. In some embodiments, the bile acid is ursodeoxycholic acid. In some embodiments, both deoxycholic acid and ursodeoxycholic acid are used. In some embodiments, the bile acid is lithocholic acid. In some embodiments, the bile acid is taurolithocholic acid. In some embodiments, the bile acid is tauroursodeoxycholic acid. In some embodiments, the bile acid is glycocholic acid.

### 4. Applications

The compounds of Formula (I) have neuroprotective effects, anti-inflammatory activity, antioxidative properties, and epithelial cell protective effects. It is hypothesised that these useful and beneficial properties may be due to the S-benzene moiety with cholestane skeleton structure of the compounds.

The compounds of Formula (I), such as HA-1 or HA-2, are therefore useful in the treatment or prevention of neurological disorders (including neurodegenerative conditions such as Alzheimer's disease, dementia caused by Alzheimer's disease, Parkinson's disease, amyotrophic lateral sclerosis (motor neurone disease), multiple sclerosis, and brain injury), the cognitive decline associated with these neurological disorders (such as memory loss), and disorders caused by stress-induced cellular damage in the inner or middle ear of a subject (such as vestibular disorders, hearing impairment, and conditions related to hair cell degeneration or hair cell death).

Thus, there is provided a pharmaceutical composition comprising a compound of Formula (I), or a pharmaceutically acceptable salt thereof, and a pharmaceutically acceptable carrier, adjuvant or diluent.

There is also provided a method of treating or preventing a neurological disorder or cognitive decline associated with a neurological disorder in a subject, the method comprising administering to the subject an effective amount of a compound of Formula (I) or a pharmaceutically acceptable salt thereof.

There is also provided a compound of Formula (I) or a pharmaceutically acceptable salt thereof for use in treating or preventing a neurological disorder or cognitive decline associated with a neurological disorder in a subject.

There is also provided the use of a compound of Formula (I) or a pharmaceutically acceptable salt thereof in the manufacture of a medicament for treating or preventing a neurological disorder or cognitive decline associated with a neurological disorder in a subject.

There is also provided a method for preventing, reducing or treating the incidence and/or severity of a disorder caused by stress-induced cellular damage in the middle or inner ear of a subject, the method comprising administering to the subject a therapeutically effective amount of a compound of Formula (I) or a pharmaceutically acceptable salt thereof.

There is also provided a method for preventing, reducing or treating the incidence and/or severity of a vestibular disorder or hearing impairment in a subject, the method comprising administering to the subject a therapeutically effective amount of a compound of Formula (I) or a pharmaceutically acceptable salt thereof.

There is also provided a method for preventing, reducing or inhibiting hair cell degeneration or hair cell death in a subject, the method comprising administering to the subject a therapeutically effective amount of a compound of Formula (I) or a pharmaceutically acceptable salt thereof.

There is also provided the use of a compound of Formula (I) or a pharmaceutically acceptable salt thereof in the manufacture of a medicament for preventing, reducing or treating the incidence and/or severity of a disorder caused by stress-induced cellular damage in the middle or inner ear of a subject.

There is also provided the use of a compound of Formula (I) or a pharmaceutically acceptable salt thereof in the manufacture of a medicament for preventing, reducing or treating the incidence and/or severity of a vestibular disorder or hearing impairment in a subject.

There is also provided the use of a compound of Formula (I) or a pharmaceutically acceptable salt thereof in the manufacture of a medicament for preventing, reducing or inhibiting hair cell degeneration or hair cell death in a subject.

There is also provided a compound of Formula (I) or a pharmaceutically acceptable salt thereof for use in preventing, reducing or treating the incidence and/or severity of a disorder caused by stress-induced cellular damage in the middle or inner ear of a subject.

There is also provided a compound of Formula (I) or a pharmaceutically acceptable salt thereof for use in preventing, reducing or treating the incidence and/or severity of a vestibular disorder or hearing impairment in a subject.

There is also provided a compound of Formula (I) or a pharmaceutically acceptable salt thereof for use in preventing, reducing or inhibiting hair cell degeneration or hair cell death in a subject.

The cellular damage in the middle or inner ear may, for example, be induced or caused by stress due to high levels of free radicals, oxidative stress, pro-inflammatory cytokines, noise-induced stress, chemically-induced stress, infection by viruses or bacteria or other infective agents, physical trauma, molecular disturbances affecting the cochlea, inflamed or damaged stria vascularis, neuronal signalling loss and apoptosis throughout the middle ear or inner ear. The cellular damage in the middle or inner ear may, for example, lead to hair cell degeneration and/or hair cell death.

The pharmaceutical compositions may also include a further active ingredient useful for treating or preventing neurological disorders, including neurodegenerative conditions such as Alzheimer's disease, dementia caused by Alzheimer's disease, Parkinson's disease, amyotrophic lateral sclerosis (motor neurone disease), multiple sclerosis, cognitive decline, such as memory loss, or brain injury, or useful for preventing, reducing or treating the incidence and/or severity of a disorder caused by stress-induced cellular damage in the middle or inner ear. The pharmaceutical compositions may also be used as medicaments for recovery and/or therapy and/or pre-treatment for ear-associated surgery for outer/middle/inner ear implanted devices (e.g., cochlear implantation and grommets).

The further active ingredient that may be usefully administered with the pharmaceutical composition include, for example, corticosteroids, antibiotics, neurotrophins, growth factors, anti-fibrotic agents, and stem cell promoting agents. In some embodiments, the further active ingredient is probucol or succinobucol.

In some embodiments, the present invention relates to maintaining, inducing, promoting, or enhancing the viability or regeneration of middle or inner ear cells, particularly middle or inner ear supporting cells and hair cells. The inventors believe that the compound of Formula (I) or a pharmaceutically acceptable salt thereof has the potential to aid recovery of hearing loss and relevant cell functions.

Whereas mechanical devices simply increase the volume, the compounds, methods, uses and compositions described herein can advantageously restore the frequency response of the ear and, unlike surgical interventions, the compounds, methods, uses and compositions described herein are non-invasive.

### 5. Pharmaceutical Compositions

The invention also provides a pharmaceutical composition comprising a compound of Formula (I) or a stereoisomer thereof, or a pharmaceutically acceptable salt thereof, and a pharmaceutically acceptable carrier.

In some embodiments, the compound of Formula (I) or a pharmaceutically acceptable salt thereof, or the pharmaceutical composition comprising the compound of Formula (I) or pharmaceutically acceptable salt thereof, may be used in combination with one or more other agents. In some embodiments, the compound of Formula (I) or a stereoisomer thereof, or a pharmaceutically acceptable salt thereof, is the sole active ingredient used in the pharmaceutical composition.

Accordingly, in some embodiments, the pharmaceutical composition may further comprise, or be administered in combination with, one or more other agents. For example, the pharmaceutical composition may further comprise, or be administered in combination with, agents useful in treating or preventing Alzheimer's disease, dementia caused by Alzheimer's disease, Parkinson's disease, amyotrophic lateral sclerosis (motor neurone disease), multiple sclerosis, cognitive decline, memory loss, or brain injury.

It will be understood that the combined administration of a compound of Formula (I) or a pharmaceutically acceptable salt thereof with the one or more other agents may be concurrent, sequential or separate administration.

In some embodiments, the compound of the Formula (I) is present in the pharmaceutical composition in an amount of from about 0.1% w/w to about 10% w/w, e.g. about 0.5 to about 5% w/w, about 5 to about 10% w/w, about 1 to about 4% w/w, about 1 to about 8% w/w, about 2 to about 7% w/w, about 3 to about 6% w/w, about 4 to about 8% w/w, or about 6 to about 10% w/w. In some embodiments, the compound of the Formula (I) is present in the pharmaceutical composition in an amount of 0.1% w/w, 0.2% w/w, 0.3% w/w, 0.4% w/w, 0.5% w/w, 0.6% w/w, 0.7% w/w, 0.8% w/w, 0.9% w/w, 1% w/w, 2% w/w, 3% w/w, 4% w/w, 5% w/w, 6% w/w, 7% w/w, 8% w/w, 9% w/w, or 10% w/w.

The term “composition” encompasses formulations comprising the compound of Formula (I) or a stereoisomer thereof, or a pharmaceutically acceptable salt thereof, with conventional carriers and excipients, and also formulations with encapsulating materials as a carrier to provide a capsule in which the compound (with or without other carriers) is surrounded by the encapsulation carrier. In pharmaceutical compositions, the carrier is “pharmaceutically acceptable” meaning that it is compatible with the other ingredients of the composition and is not deleterious to a subject. The pharmaceutical compositions may contain other agents or further active agents as described above, and may be formulated, for example, by employing conventional solid or liquid vehicles or diluents, as well as pharmaceutical additives of a type appropriate to the mode of desired administration (for example, excipients, binders, preservatives, stabilizers, flavours, etc.) according to techniques such as those well known in the art of pharmaceutical formulation (See, for example, Remington: The Science and Practice of Pharmacy, 21st Ed., 2005, Lippincott Williams & Wilkins).

The pharmaceutical composition may be suitable for oral, rectal, nasal, topical (including dermal, buccal and sub-lingual), or parenteral (including intramuscular, subcutaneous and intravenous) administration.

The compound of Formula (I) or a stereoisomer thereof, or a pharmaceutically acceptable salt thereof, together with a conventional adjuvant, carrier, or diluent, may thus be placed into the form of pharmaceutical compositions and unit dosages thereof. The pharmaceutical composition may be a solid, such as a tablet or filled capsule, or a liquid such as solution, suspension, emulsion, elixir, or capsule filled with the same, for oral administration. The pharmaceutical composition may also be in the form of suppositories for rectal administration or in the form of sterile injectable solutions for parenteral (including subcutaneous) use.

Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

For preparing pharmaceutical compositions from the compounds of Formula (I) or a stereoisomer thereof, or a pharmaceutically acceptable salt thereof, pharmaceutically acceptable carriers can be either solid or liquid. Solid form preparations include powders, tablets, pills, capsules, cachets, suppositories, and dispensable granules. A solid carrier can be one or more substances which may also act as diluents, flavouring agents, solubilisers, lubricants, suspending agents, binders, preservatives, tablet disintegrating agents, or an encapsulating material.

Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Tablets, powders, capsules, pills, cachets, and lozenges can be used as solid forms suitable for oral administration.

Liquid form preparations include solutions, suspensions, and emulsions, for example, water or water-propylene glycol solutions. For example, parenteral injection liquid preparations can be formulated as solutions in aqueous polyethylene glycol solution.

Solutions or suspensions may include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH may be adjusted with acids or bases, such as hydrochloric acid or sodium hydroxide. The parenteral preparation may be enclosed in ampoules, disposable syringes or multiple dose vials made of glass or plastic.

Sterile liquid form compositions include sterile solutions, suspensions, emulsions, syrups and elixirs. The active ingredient can be dissolved or suspended in a pharmaceutically acceptable carrier, such as sterile water, sterile organic solvent or a mixture of both.

The pharmaceutical compositions according to the present invention may thus be formulated for parenteral administration (e. g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

Pharmaceutical forms suitable for injectable use include sterile injectable solutions or dispersions, and sterile powders for the extemporaneous preparation of sterile injectable solutions. They should be stable under the conditions of manufacture and storage and may be preserved against oxidation and the contaminating action of microorganisms such as bacteria or fungi.

The solvent or dispersion medium for the injectable solution or dispersion may contain any of the conventional solvent or carrier systems for injectable solutions or dispersions, and may contain, for example, water, ethanol, polyol (for example, glycerol, propylene glycol and liquid polyethylene glycol, and the like), suitable mixtures thereof, and vegetable oils.

Pharmaceutical forms suitable for injectable use may be delivered by any appropriate route including intravenous, intramuscular, intracerebral, intrathecal, epidural injection or infusion.

Sterile injectable solutions are prepared by incorporating the active ingredient in the required amount in the appropriate solvent with various other ingredients such as those enumerated above, as required, followed by filtered sterilization. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile-filtered solution of the active ingredient plus any additional desired ingredients.

For intravenous administration, suitable carriers include physiological saline, bacteriostatic water, Cremophor ELTM (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier may be a solvent or dispersion medium containing, for example, water, ethanol, polyol (for example, glycerol, propylene glycol, and liquid polyetheylene glycol, and the like), and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions may be brought about by including in the composition an agent that delays absorption, for example, aluminium monostearate and gelatin.

The compounds of Formula (I) or a stereoisomer thereof, or a pharmaceutically acceptable salt thereof may be formulated into compositions suitable for oral administration, for example, with an inert diluent or with an assimilable edible carrier, or enclosed in hard or soft shell gelatin capsule, or compressed into tablets, or incorporated directly with the food of the diet. For oral therapeutic administration, the active compound may be incorporated with excipients and used in the form of ingestible tablets, buccal tablets, troches, capsules, elixirs, suspensions, syrups, wafers, and the like.

The amount of active compound in therapeutically useful compositions should be sufficient that a suitable dosage will be obtained.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active ingredient(s) may be incorporated into sustained-release preparations and formulations, including those that allow specific delivery of the active ingredient to specific regions of the gut.

Aqueous solutions suitable for oral use can be prepared by dissolving the active component in water and adding suitable colorants, flavours, stabilising and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Pharmaceutically acceptable carriers include any and all pharmaceutically acceptable solvents, dispersion media, coatings, antibacterial and antifungal agents, isotonic and absorption delaying agents and the like.

Also included are solid form preparations that are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilising agents, and the like.

For topical administration to the epidermis, the compounds of Formula (I) or a stereoisomer thereof, or a pharmaceutically acceptable salt thereof, may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions for nasal administration may be applied directly to the nasal cavity by conventional means, for example with a dropper, pipette or spray. The compositions may be provided in single or multidose form. In the case of a dropper or pipette, this may be achieved by the patient administering an appropriate, predetermined volume of the solution or suspension. In the case of a spray, this may be achieved for example by means of a metering atomising spray pump. To improve nasal delivery and retention the compounds of Formula (I) or a stereoisomer thereof may be encapsulated with cyclodextrins, or formulated with other agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas.

The aerosol may conveniently also contain a surfactant such as lecithin. The dose of the active ingredient may be controlled by provision of a metered valve.

Alternatively, the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g. gelatin, or blister packs from which the powder may be administered by means of an inhaler.

In formulations intended for administration to the respiratory tract, including intranasal formulations, the active ingredient will generally have a small particle size for example of the order of 5 to 10 microns or less. Such a particle size may be obtained by means known in the art, for example by micronisation.

When desired, compositions adapted to give sustained release of the active ingredient may be employed.

In some embodiments, the pharmaceutical composition comprising the compound of Formula (I) or a stereoisomer thereof, or a pharmaceutically acceptable salt, thereof is administered, or is formulated to be administered, to the outer, middle or inner ear by a method described below:
1) Application of the composition, e.g. ear drops, to the outer ear such that the composition moves through the ear canal to drain into the middle ear and subsequently into the inner ear;
2) Transtympanic (TT) syringe delivery of the pharmaceutical composition to the middle ear as a bolus (0.1 to 1 mL);
3) TT syringe delivery of the pharmaceutical composition specifically to the round window (RW) as a small gel or capsule (e.g. about 0.05 to 1 mL), e.g. a single transtympanic dose injected to the round window, or multiple transtympanic doses injected to the round window;
4) TT syringe delivery of the pharmaceutical composition specifically to the oval window as a gel (e.g. about 0.05 to 1 mL);
5) TT delivery of the pharmaceutical composition to the round window via a microwick or other catheter device;
6) TT and trans-RW delivery of the pharmaceutical composition directly into the inner ear fluids via a needle;
7) TT delivery of the pharmaceutical composition via a defect in the tympanic membrane (such as a grommet or perforation) with the pharmaceutical composition delivered to the ear canal;
8) Transmastoid or transantral delivery of the pharmaceutical composition which then drains into the middle ear and subsequently into the inner ear;
9) Direct delivery into the inner ear fluids, with the pharmaceutical composition released from a ‘tube’, which is inserted into the cochlea;
10) Systemic delivery (coupled with methods of enhancing inner ear uptake) either via an intravenous, intramuscular, oral route or a combination of these;
11) Direct injection into the inner ear via the round window or cochleostomy with or without a catheter or device (such as a cochlear implant);
12) Direct injection into the inner ear via the inner ear appendages such as injection into the endolymphatic sac, semicircular canal, or vestibule with or without a catheter or device;
13) Direct injection into the inner ear via the oval window such as during the stapedotomy or stapedotomy, or via a middle ear prosthesis (such as a stapes prosthesis, or middle ear implant).

The above-described methods are known in the art. For example, drug delivery systems are described in the literature, e.g. M. Peppi, A. Marie, C. Belline & J. T. Borenstein (2018), “Intracochlear drug delivery systems: a novel approach whose time has come”, Expert Opinion on Drug Delivery, 15:4, 319-324; J. Wang and J L. Puel, “Presbycusis: An Update on Cochlear Mechanisms and Therapies”, J. Clin. Med. 2020, 9, 218; J. Patel, M. Szczupak, S. Rajguru, C. Balaban and M. E. Hoffer (2019), “Inner Ear Therapeutics: An Overview of Middle Ear Delivery”, Front. Cell. Neurosci. 13:261; S. Nyberg, N. J. Abbott, X. Shi, P. S. Steyger, A. Dabdoub, “Delivery of therapeutics to the inner ear: The challenge of the blood-labyrinth barrier” Sci. Transl. Med. 11, eaao0935 (2019); A. A. McCall, E. E. Leary Swan, J. T. Borenstein, W. F. Sewell, S. G. Kujawa, and M. J. McKenna, “Drug Delivery for Treatment of Inner Ear Disease: Current State of Knowledge”, Ear Hear., 2010 April; 31(2): 156-165; K. Mader, E. Lehner, A. Liebau, S. K. Plontke, “Controlled drug release to the inner ear: Concepts, materials, mechanisms, and performance”, Hearing Research, 368 (2018) 49-66; Yutian Ma, Andrew K. Wise, Robert K. Shepherd, and Rachael T. Richardson, “New molecular therapies for the treatment of hearing loss”, Pharmacology & Therapeutics 200 (2019), 190-209; (each of these documents is incorporated herein by reference).

In some embodiments, the pharmaceutical composition is administered by one of the modes of delivery listed below:
incorporated into or used in conjunction with tympanic membrane graft material as part of tympanoplasty or myringoplasty surgery;
incorporated into middle ear prostheses as part of ossicular chain reconstructive surgery;
used with established middle ear packing material such as Gelfoam or Gelfilm;
used as part of irrigation solution during middle ear or inner ear surgical procedures;
incorporated with suture material;
incorporated into ventilation tubes (grommets);
incorporated into bone condition implants such as the BAHA, Bonebridge, Ossia or Ponto device; or
incorporated into microsurgical drills.

In some embodiments, the pharmaceutical composition is administered into the middle ear by application of a liquid or gel formulation by bolus transtympanic injection using, for example, a microfluidic device such as a fine syringe in a volume range of millilitres or microlitres. In some embodiments, the volume administered for a liquid or gel formulation is about 0.05 mL to about 1 mL. For example, the volume administered may be about 0.1 mL to about 0.8 mL, about 0.2 mL to about 0.7 mL, about 0.3 mL to about 0.6 mL, or about 0.4 mL to about 0.5 mL.

In some embodiments, the pharmaceutical composition is administered by application of a liquid or gel formulation directly onto the round window or oval window membrane(s). Application to these membranes may be accomplished using methods known in the art, e.g., intratympanic injection of a liquid or gel formulation, e.g., using a microfluidic device such as a fine syringe in a volume range of millilitres or microlitres. In some embodiments, the volume administered for a liquid or gel formulation is about 0.05 mL to about 1 mL. For example, the volume administered may be about 0.1 mL to about 0.8 mL, about 0.2 mL to about 0.7 mL, about 0.3 mL to about 0.6 mL, or about 0.4 mL to about 0.5 mL.

In some embodiments, the pharmaceutical composition is administered by application of a liquid formulation by a catheter or wick delivery system. Catheter or wick delivery systems are known in the art. For example, such systems are described in Silverstein, H., Thompson, J., Rosenberg, S. I., Brown, N., Light, J., 2004. “Silverstein MicroWick”, Otolaryngol. Clin. North Am., 37, 1019-1034, which is incorporated herein by reference.

In some embodiments, the pharmaceutical composition is administered directly to the middle or inner ear by a drug delivery system comprising the drug embedded in a silicone carrier such as a cochlear implant or middle ear implant. Drug delivery systems with active ingredients embedded in a silicone carrier are known in the art. Such systems are discussed in Plontke S K, Götze G, Rahne T, Liebau A. Intracochlear drug delivery in combination with cochlear implants: Current aspects. HNO. 2017; 65(Suppl 1):19-28, which is incorporated herein by reference. A specific example with animal data is discussed in Farhadi M, Jalessi M, Salehian P, et al. Dexamethasone eluting cochlear implant: Histological study in animal model. Cochlear Implants Int. 2013; 14(1):45-50, which is incorporated herein by reference. A more recent example with human subjects is discussed in Briggs R, O'Leary S, Birman C, et al. Comparison of electrode impedance measures between a dexamethasone-eluting and standard Cochlear™ Contour Advance® electrode in adult cochlear implant recipients. Hear Res. 2020; 390:107924, which is incorporated herein by reference.

In some embodiments, the pharmaceutical composition is administered directly to the middle or inner ear by a drug delivery system comprising the drug given in a droplet form through a defect in the tympanic membrane including through a ventilation tube. Drug delivery systems through a tympanic membrane defect are known in the art.

Therefore, in preferred embodiments, the pharmaceutical composition is administered by the transtympanic route, e.g., using a microfluidic device such as a fine syringe in a volume range of millilitres or microlitres. In some embodiments, the volume administered for a liquid or gel formulation is about 0.05 mL to about 1 mL. For example, the volume administered may be about 0.1 mL to about 0.8 mL, about 0.2 mL to about 0.7 mL, about 0.3 mL to about 0.6 mL, or about 0.4 mL to about 0.5 mL.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

It is especially advantageous to formulate parenteral compositions in dosage unit form for ease of administration and uniformity of dosage. Parental compositions may be in the form of physically discrete units suited as unitary dosages for the subjects to be treated, each unit containing a predetermined quantity of the active ingredient calculated to produce the desired therapeutic effect in association a pharmaceutical carrier.

The compounds may also be administered in the absence of carrier where the compounds are in unit dosage form.

Compositions comprising compounds of Formula (I) or a stereoisomer thereof, or a pharmaceutically acceptable salt thereof, formulated for oral delivery either alone or in combination with another agent are particularly preferred.

When the pharmaceutical composition is in the form of a solution, a mixture, a suspension, microparticles, nanoparticles, or a gel, the concentration of the compound of Formula (I) or a pharmaceutically acceptable salt thereof may, for example, be from 0.1 to 1000 mg/mL, e.g. from 0.1 to 2 mg/mL, from 0.2 to 5 mg/mL, from 1 to 5 mg/mL, from 2 to 8 mg/mL, from 5 to 10 mg/mL, from 10 to 20 mg/mL, from 20 to 30 mg/mL, from 30 to 40 mg/mL, from 40 to 50 mg/mL, from 50 to 60 mg/mL, from 60 to 70 mg/mL, from 70 to 80 mg/mL, from 80 to 90 mg/mL, from 90 to 100 mg/mL, from 10 to 100 mg/mL, from 100 to 200 mg/mL, from 200 to 300 mg/mL, from 300 to 400 mg/mL, from 500 to 1000 mg/mL, from 400 to 500 mg/mL, from 500 to 600 mg/mL, from 600 to 700 mg/mL, from 700 to 800 mg/mL, from 800 to 900 mg/mL, or from 900 to 1000 mg/mL of the compound of Formula (I) or a pharmaceutically acceptable salt thereof.

The pharmaceutical compositions comprising the compound of Formula (I) or a pharmaceutically acceptable salt thereof are formulated to be compatible with the intended route of administration.

In one embodiment, the pharmaceutical composition comprises a cyclodextrin (e.g. (2-hydroxypropyl)-β-cyclodextrin) and the compound of Formula (I) or a pharmaceutically acceptable salt thereof in a form suitable for cochlea (inner ear) delivery. For example, the composition may be a gel formulation suitable for cochlea (inner ear) delivery. Thus, in some embodiments, the composition is formulated to be administered to the middle or inner ear. The compound of Formula (I) or a pharmaceutically acceptable salt thereof may be in the form of microparticles. The gel formulation may be an ultrasonic gel suitable for cochlea (inner ear) delivery.

In some embodiments, the pharmaceutical composition further comprises a bile acid or bile salt, or a derivative or metabolite thereof. Bile acids are facial amphiphiles, i.e. they contain both hydrophobic and hydrophilic faces. Being facial amphiphiles enables bile acids to be employed in drug delivery systems for selective drug-targeting to the liver or to enhance drug bioavailability by improving intestinal absorption and metabolic stability. Bile acid-based drug delivery systems, in the form of mixed micelles, bilosomes and drug conjugates, are versatile nanocarriers. It is also believed that the steroidal backbone conserves lipophilicity and hence leads to permeation-enhancing effects, which is advantageous, for example, when the composition is for administration to the middle or inner ear.

Any bile acid or bile salt, or a derivative or metabolite thereof, may be used to enhance the bioavailability of the compound of Formula (I) or a stereoisomer thereof, or a pharmaceutically acceptable salt thereof, and may be in the form of bile acid-based nanoparticles or bile acid-based microparticles. For example, the bile acid or bile salt may be a primary bile acid, or a secondary bile acid or any bile acid with an amino acid moiety attached. Various bile acids and bile salts are commercially available.

In some embodiments, the pharmaceutical composition may further comprise one or more of cholic acid, deoxycholic acid (DCA), chenodeoxycholic acid, ursodeoxycholic acid (UDCA; also known as ursodiol), lithocholic acid, muricholic acid (α-muricholic acid, β-muricholic acid, γ-muricholic acid or ω-muricholic acid), murideoxycholic acid, hypochloric acid (α-hypochloric acid or β-hypochloric acid), hyodeoxycholic acid, ursocholic acid, lagocholic acid, lagodeoxycholic acid, cricetocholic acid, vulpecholic acid, glycocholic acid, glycochenodeoxycholic acid, taurocholic acid, taurochenodeoxycholic acid, glycodeoxycholic acid, taurodeoxycholic acid, glycolithocholic acid, taurolithocholic acid, tauroursodeoxycholic acid, obeticholic acid, glycomuricholic acid (α-glycomuricholic acid, β-glycomuricholic acid, γ-glycomuricholic acid, ω-glycomuricholic acid), or tauromuricholic acid (α-tauromuricholic acid, β-tauromuricholic acid, γ-tauromuricholic acid, ω-tauromuricholic acid), or a salt, derivative or metabolite thereof.

In some embodiments, the bile acid is deoxycholic acid and/or ursodeoxycholic acid. In some embodiments, the bile acid is deoxycholic acid. In some embodiments, the bile acid is ursodeoxycholic acid. In some embodiments, both deoxycholic acid and ursodeoxycholic acid are used. In some embodiments, the bile acid is lithocholic acid. In some embodiments, the bile acid is taurolithocholic acid. In some embodiments, the bile acid is tauroursodeoxycholic acid. In some embodiments, the bile acid is glycocholic acid. In some embodiments, the bile acid is chenodeoxycholic acid, taurocholic acid or cholic acid.

In some embodiments, the bile acid or bile salt, or a derivative or metabolite thereof, is present in the pharmaceutical composition in an amount of from about 0.1% w/w to about 10% w/w, e.g. about 0.5 to about 5% w/w, about 5 to about 10% w/w, about 1 to about 4% w/w, about 1 to about 8% w/w, about 2 to about 7% w/w, about 3 to about 6% w/w, about 4 to about 8% w/w, or about 6 to about 10% w/w. In some embodiments, the bile acid or bile salt is present in the pharmaceutical composition in an amount of 0.1% w/w, 0.2% w/w, 0.3% w/w, 0.4% w/w, 0.5% w/w, 0.6% w/w, 0.7% w/w, 0.8% w/w, 0.9% w/w, 1% w/w, 2% w/w, 3% w/w, 4% w/w, 5% w/w, 6% w/w, 7% w/w, 8% w/w, 9% w/w, or 10% w/w.

In some embodiments, a pharmaceutical composition in the form of a gel may include the following components: the compound of Formula (I) or a pharmaceutically acceptable salt thereof, one or more cyclodextrins, polysorbate 80 (Tween 80), glycerol, propylene glycol, a water-soluble gel, and water.

For example, the pharmaceutical composition may include the following components:

| Component | Amount |
|---|---|
| Water | about 200-500 mL |
| (2-Hydroxypropyl)-β-cyclodextrin | about 1-15 g |
| Polysorbate 80 (Tween 80) | about 1-10 mL |
| Glycerol | about 1-10 mL |
| Propylene glycol | about 1-20 mL |
| Metron Water Soluble Gel | about 1-20 g |
| Compound of Formula (I) or a | about 0.1-10 g |
| pharmaceutically acceptable salt | |
| thereof (e.g. HA-1 or HA-2) | |
| Deoxycholic acid (DCA) | about 0.1-10 g |
| Ursodeoxycholic acid (UDCA) | about 0.1-10 g |

### Two Specific Composition Examples are Provided Below:

| Component | Amount | Amount |
|---|---|---|
| Water | about 350 mL | about 350 mL |
| (2-Hydroxypropyl)-β-cyclodextrin | about 7 g | about 7 g |
| Polysorbate 80 (Tween 80) | about 5 mL | about 5 mL |
| Glycerol | about 6 mL | about 5 mL |
| Propylene glycol | about 12 mL | about 11 mL |
| Metron Water Soluble Gel | about 6.5 g | about 6 g |
| HA-1 or HA-2 | about 4 g | about 4 g |
| Deoxycholic acid (DCA) | about 3 g | about 2 g |
| Ursodeoxycholic acid (UDCA) | about 1 g | about 3 g |

A typical method of preparation is as follows. A mixture of (2-hydroxypropyl)-β-cyclodextrin in water is mixed with stirring at about 80° C. for about 1 hour. The compound of Formula (I) or a pharmaceutically acceptable salt thereof is then added and the resulting mixture is heated with stirring to about 80° C. for about 1 hour. Tween 80 is then added, and the resulting mixture is heated with stirring to about 80° C. for about 1 hour. Ursodeoxycholic acid (UDCA) is then added, and the resulting mixture is heated with stirring to about 80° C. for about 1 hour. Deoxycholic acid is then added, and the resulting mixture is heated with stirring to about 80° C. for about 1 hour. Metron Water Soluble Gel is then added, and the resulting mixture is heated with stirring to about 80° C. for about 1 hour. Glycerol and propylene glycol are then added, and the resulting mixture is heated with stirring to about 80° C. for about 1 hour. The resulting mixture is then stirred at about 80° C. for a further hour, and then water is added to make up the volume to the desired amount.

The pharmaceutical compositions may be included in a container, pack, or dispenser together with instructions for administration.

In a further embodiment, the pharmaceutical composition comprising the compound of Formula (I) or a pharmaceutically acceptable salt thereof may comprise at least one further active ingredient. The further active ingredient may be an antibacterial agent, an antiviral agent, an antifungal agent, an anti-inflammatory agent, an osmotically active substance (e.g. mannitol), or other suitable therapeutically or pharmacologically active agent. In some embodiments, the further active ingredient is a steroid, for example, selected from dexamethasone, methylprednisolone and prednisolone. In some embodiments, the further active ingredient is an antibiotic, such as gentamicin. In some embodiments the further active ingredient may have otoprotective effects, specific to preventing middle or inner ear damage related to chemotherapy, such as that which occurs with cisplatin treatment or radiotherapy.

In a further embodiment, the pharmaceutical composition comprising the compound of Formula (I) or a pharmaceutically acceptable salt thereof may be administered simultaneously, separately or sequentially in combination with at least one further active ingredient, also following different routes of administration for each active ingredient. The further active ingredient may be an antibacterial agent, an antiviral agent, an antifungal agent, an anti-inflammatory agent, a chemotherapeutic agent, an osmotically active substance (e.g. mannitol), or other suitable therapeutically or pharmacologically active agent. In particular, the further active ingredient may be an ototoxic or oto-irritant therapeutic drug; commonly used ototoxic drugs include aminoglycosides, platinum-based chemotherapeutic agents, loop diuretics, macrolide antibiotics, and antimalarials. In some embodiments, the further active ingredient is a steroid, for example, selected from dexamethasone, methylprednisolone and prednisolone.

In some embodiments, the pharmaceutical composition comprising the compound of Formula (I) or a pharmaceutically acceptable salt thereof may be administered to a subject being treated with gene therapy (e.g. viral vectors), or stem cell therapy.

Where appropriate, following treatment, the subject may be tested for an improvement in, for example, hearing or other symptoms related to middle or inner ear disorders. Methods for measuring hearing are well-known and include pure tone audiometry, air conduction, and bone conduction tests. These exams measure the limits of loudness (intensity) and pitch (frequency) that a subject can hear. Hearing tests in humans include behavioural observation audiometry (for infants to seven months), visual reinforcement orientation audiometry (for children 7 months to 3 years); play audiometry for children older than 3 years; and standard audiometric tests for older children and adults, e.g., whispered speech, pure tone audiometry; tuning fork tests; brain stem auditory evoked response (BAER) testing or auditory brain stem evoked potential (ABEP) testing. Oto-acoustic emission testing may be used to test the functioning of the cochlear hair cells, and electro-cochleography provides information about the functioning of the cochlea and the first part of the nerve pathway to the brain. In some embodiments, treatment can be continued with or without modification or can be stopped.

In some embodiments, the composition comprises a further substance that is a permeability enhancer.

Advantageously, the permeability enhancer enhances permeation through the middle ear (tympanic membrane) into the round window and inner ear (cochlea, Organ of Corti, Scala vestibule, Scala tympani and Scala media). Drug permeation in sufficient amounts deep into the cochlea is one of the major challenges of current commercial gels. Additionally, the permeability enhancer may function as a stabilising agent, or as a component that may provide slow or controlled release of the active ingredient.

In some embodiments, the pharmaceutical compositions comprising such permeability enhancers will facilitate the delivery of the composition across biological barriers that separate the middle and inner ear, e.g., the round window, thereby efficiently delivering a therapeutically effective amount of the pharmaceutical composition to the inner ear. Efficient delivery to the cochlea, Organ of Corti, and/or vestibular organs is desired because these tissues host the support cells that promote sensory hair cell regeneration when treated or contacted with compositions described herein.

In some embodiments, the further permeability enhancer is selected from a polyol such as polyethylene glycol (PEG), glycerol (glycerin), maltitol, sorbitol, etc.; diethylene glycol monoethyl ether, azone, benzalkonium chloride (ADBAC), cetylperidium chloride, cetylmethylammonium bromide, dextran sulfate, lauric acid, menthol, methoxy salicylate, oleic acid, phosphatidylcholine, polyoxyethylene, polysorbate 80, sodium glycolate, sodium lauryl sulfate, sodium salicylate, sodium taurocholate, sodium taurodeoxycholate, sulfoxides, sodium deoxycholate, sodium glycodeoxycholate, sodium taurocholate and surfactants such as sodium lauryl sulfate, laureth-9, cetylpyridinium chloride and polyoxyethylene monoalkyl ethers, benzoic acids, such as sodium salicylate and methoxy salicylate, fatty acids, such as lauric acid, oleic acid, undecanoic acid and methyl oleate, fatty alcohols, such as octanol and nonanol, laurocapram, cyclodextrins, thymol, limonene, urea, chitosan and other natural and synthetic polymers.

In some embodiments, the further permeability enhancer is present in the pharmaceutical composition in an amount of from about 0.1% w/w to about 10% w/w, e.g. about 0.5 to about 5% w/w, about 5 to about 10% w/w, about 1 to about 4% w/w, about 1 to about 8% w/w, about 2 to about 7% w/w, about 3 to about 6% w/w, about 4 to about 8% w/w, or about 6 to about 10% w/w. In some embodiments, the permeability enhancer is present in the pharmaceutical composition in an amount of 0.1% w/w, 0.2% w/w, 0.3% w/w, 0.4% w/w, 0.5% w/w, 0.6% w/w, 0.7% w/w, 0.8% w/w, 0.9% w/w, 1% w/w, 2% w/w, 3% w/w, 4% w/w, 5% w/w, 6% w/w, 7% w/w, 8% w/w, 9% w/w, or 10% w/w.

In some embodiments, the further permeability enhancer is a cyclic oligosaccharide, which consists of a macrocyclic ring of glucose subunits joined by α-1,4 glycosidic bonds.

In some embodiments, the cyclic oligosaccharide is a cyclodextrin. Cyclodextrins are a family of cyclic oligosaccharides, consisting of a macrocyclic ring composed of five or more α-D-glucopyranoside units joined by α-1,4 glycosidic bonds. Typical cyclodextrins contain a number of glucose monomers ranging from six to eight units in a ring: α-cyclodextrin (6 glucose subunits), β-cyclodextrin (7 glucose subunits), γ-cyclodextrin (8 glucose subunits). Any cyclodextrin or a derivative thereof that enhances permeability may be used.

In some embodiments, the cyclodextrin may be an α-cyclodextrin, a β-cyclodextrin, or a γ-cyclodextrin, or a larger polymerised cyclodextrin. For example, the cyclodextrin may be (2-hydroxypropyl)-β-cyclodextrin.

Cyclodextrins have a hydrophobic interior and hydrophilic exterior, and form complexes with hydrophobic compounds such as hydrophobic active ingredients (e.g. probucol). Advantageously, cyclodextrins may confer solubility and stability to the active ingredient, enabling the complexes of cyclodextrins with a hydrophobic active ingredient to be able to penetrate body tissues and release the active ingredient.

In some embodiments, the cyclodextrin is present in the pharmaceutical composition in an amount of from about 1% w/w to about 50% w/w. For example, the cyclodextrin may be present in the pharmaceutical composition in an amount of from about 1% w/w to about 25% w/w, e.g. from about 2% w/w to about 20% w/w, from about 3% w/w to about 15% w/w, or from about 5% w/w to about 10% w/w.

### Pharmaceutically Acceptable Carriers for Administration to the Ear

In some embodiments, the pharmaceutical composition comprises a pharmaceutically acceptable carrier. The pharmaceutically acceptable carrier is selected depending on the mode of administration of the pharmaceutical composition, e.g. transtympanic administration. In advantageous embodiments, the pharmaceutically acceptable carrier is not perceived as foreign by the ear.

In some embodiments, the pharmaceutically acceptable carrier is water. Typically, water is a component of the pharmaceutical composition. In some embodiments, the water is deionised water or distilled water.

In some embodiments, the pharmaceutically acceptable carrier comprises a polymer, e.g., a hydrogel (a water-soluble gel), that provides local and sustained release of the active ingredient (e.g. bile acid-conjugate of Formula (I)). Such polymers and hydrogels are known in the art and are suitable for transtympanic administration. In some embodiments, the viscosity of the gel is 1-100 mPa S.

Examples of polymers and hydrogels suitable for transtympanic administration include: thermo-reversible triblock copolymer poloxamer 407 (see, e.g., Wang et al., Audiol Neurootol. 2009; 14(6):393-401. Epub 2009 Nov. 16; and Wang et al., Laryngoscope. 2011 February; 121(2):385-91); poloxamer-based hydrogels; Pluronic F-127 (see, e.g., Escobar-Chavez et al., J Pharm Pharm Sci. 2006; 9(3):339-5); Pluronic F68, F88, or F108; polyoxyethylene-polyoxypropylene triblock copolymer (e.g., a polymer composed of polyoxypropylene and polyoxyethylene, of general formula E106 P70 E106; see GB2459910, US20110319377 and US20100273864); MPEGPCL diblock copolymers (Hyun et al., Biomacromolecules. 2007 April; 8(4):1093-100. Epub 2007 Feb. 28); hyaluronic acid hydrogels (Borden et al., Audiol Neurootol. 2011; 16(1):1-11); gelfoam cubes (see, e.g., Havenith et al., Hearing Research, February 2011; 272(1-2):168-177); and gelatin hydrogels (see, e.g., Inaoka et al., Acta Otolaryngol. 2009 April; 129(4):453-7). Other biodegradable, biocompatible polymers may be used, such as ethylene vinyl acetate, polyanhydrides, polyglycolic acid, collagen, polyorthoesters, and polylactic acid. Tunable self-assembling hydrogels made from natural amino acids L and D may also be used, e.g., as described in Hauser et al e.g. Ac-LD6-COOH (L) e.g. Biotechnol Adv. 2012 May June; 30(3):593-603. Such compositions may be prepared using standard techniques, or obtained commercially, e.g., from Alza Corporation and Nova Pharmaceuticals, Inc.

In some embodiments, the pharmaceutically acceptable carrier is selected from one or more of glycerol, propylene glycol, polysorbate 80 (Tween 80), gels (e.g. water-soluble gels), hyaluronic acid, polyvinyl alcohol (PVA), poly-lactic-co-glycolic acid (PLGA), and PEG400. Thus, in some embodiments, the pharmaceutically acceptable carrier comprises any two or more of these carriers in combination. Such carriers are suitable for transtympanic administration.

In some embodiments, the pharmaceutically acceptable carrier is selected from glycerol, propylene glycol, and polysorbate 80 (Tween 80). In some embodiments, the pharmaceutically acceptable carrier is glycerol. In some embodiments, the pharmaceutically acceptable carrier is propylene glycol. In some embodiments, the pharmaceutically acceptable carrier is polysorbate 80 (Tween 80). In some embodiments, the pharmaceutically acceptable carrier is a water-soluble gel. In some embodiments, the pharmaceutical composition comprises glycerol, propylene glycol, polysorbate 80 (Tween 80), and a water-soluble gel.

In some embodiments, the water-soluble gel is selected from a thermo-sensitive gel or an adhesive sol-gel transition hydrogel. Commercially available water-soluble gels suitable for use in the pharmaceutical composition described herein include, for example, Metron water-soluble ultrasonic gel, Pluronic F127 and hyaluronic acid (HA).

Accordingly, in some embodiments, the pharmaceutical composition may comprise an active ingredient (e.g. bile acid-conjugate of Formula (I)) and any one or more of the following:
1: Polyethylene glycol (of any molecular weight) and any derivative thereof;
2: Polyvinylpyrrolidone (of any molecular weight) and any derivative thereof;
3: Polytetrafluoroethylene (PTFE; Teflon) (of any molecular weight) and any derivative thereof;
4: Poly-L-Ornithine (of any molecular weight) and any derivative thereof;
5: Polystyrene sulphonate (of any molecular weight) and any derivative thereof;
6: Acacia (of any molecular weight);
7: Starch (of any molecular weight);
8: Chitosan (of any molecular weight);
9: Poly-L-lysine (of any molecular weight) and any derivative thereof;
10: Poly-allyl-amine (of any molecular weight) and any derivative thereof;
11: Gelatin (of any molecular weight) and any derivative thereof;
12: Pectin (of any molecular weight) and any derivative thereof;
13: Poly (vinyl alcohol) (of any molecular weight) and any derivative thereof;
14: Poloxamer 407;
15: Polysorbate of any polyoxyethylene number and configuration;
16: Pluronic F127;
17: Hyaluronic acids (of any molecular weight) and any derivative thereof;
18: Alginates of any G and M unit varieties/combinations and any derivative thereof;
19: Bile acids: including salts with any cation as well as conjugation with any amino acid (natural, synthetic and derivatives of);
20: Spiroquinone: (chemical name: 2,4,9,11-tetrakis(1,1-dimethylethyl)-14,14-dimethyl-13,15-dithiadispiro[5.0.5.3]pentadeca-1,4,8,11-tetraene-3,10-dione);
21: All cyclodextrins and derivates thereof: alpha-, beta-, and gamma-cyclodextrin and any of their derivatives: any cyclodextrin including any member of the family of cyclic oligosaccharides, consisting of a macrocyclic ring of glucose subunits joined by α-1,4 glycosidic bonds. These include all cyclodextrins composed of 5 or more α-D-glucopyranoside units linked 1→4, as in amylose with any number of glucose monomers (as an example: α (alpha)-cyclodextrin: 6 glucose subunits, β (beta)-cyclodextrin: 7 glucose subunits and γ (gamma)-cyclodextrin: 8 glucose subunits) and also all their derivatives including any modification to their hydroxyl group (including (2-Hydroxypropyl)-β-cyclodextrin and all derivatives thereof);
22: Propylene glycol: propane-1,2-diol; or
23: Triethanolamine.

### Dosages

An “effective amount” or “therapeutically effective amount” is an amount sufficient to effect a beneficial or desired therapeutic effect. This amount may be the same or different from a “prophylactically effective amount”, which is an amount necessary to prevent onset of the disorder or disorder symptoms. An effective amount may be administered in one or more administrations, applications or dosages. A therapeutically effective amount of a therapeutic compound (i.e., an effective dosage) depends on the therapeutic compounds selected.

An appropriate dosage level of a compound of Formula (I) or a stereoisomer thereof, or a pharmaceutically acceptable salt thereof, administered to a subject will generally be about 0.01 to 500 mg per kg subject body weight per day which can be administered in single or multiple doses.

It will be understood that the specific dose level and frequency of dosage for any particular subject may be varied and will depend upon a variety of factors including the activity of the specific compound employed, the metabolic stability and length of action of that compound, the age, body weight, general health, sex, genetics and diet of the subject, the mode and time of administration, rate of excretion, drug combinations, and the severity of the particular condition.

Suitable dosages of the compound of Formula (I) or further active agents administered in combination with compound of Formula (I) can be readily determined by a person skilled in the art having regard to the particular compound of Formula (I) or further active agent selected.

It will further be understood that when the compound of Formula (I) or a stereoisomer thereof, or a pharmaceutically acceptable salt thereof, are to be administered in combination with one or more agents, or other active agents, the dosage forms and levels may be formulated for either concurrent, sequential or separate administration or a combination thereof.

The subject may be a human subject, intended to comprise both adults and the “pediatric population” (where the term “pediatric population” is understood as the part of the population ranging from birth to eighteen years of age).

The following examples are included to increase the understanding of the invention, without having any limiting effect of the invention.

### EXAMPLES

The present invention is further described below by reference to the following non-limiting Examples.

### Materials and Methods

All chemical reagents were purchased from commercial sources (Sigma Aldrich, Scharlab SL and Merck & Co, Inc.) and used without further purification.

### (4R)-4-[(3R,5R,8R,9S,10S,13R,14S,17R)-3-acetoxy-10,13-dimethyl-2,3,4,5,6,7,8,9,11,12,14,15,16,17-tetradecahydro-1H-cyclopenta[a]phenanthren-17-yl]pentanoic Acid

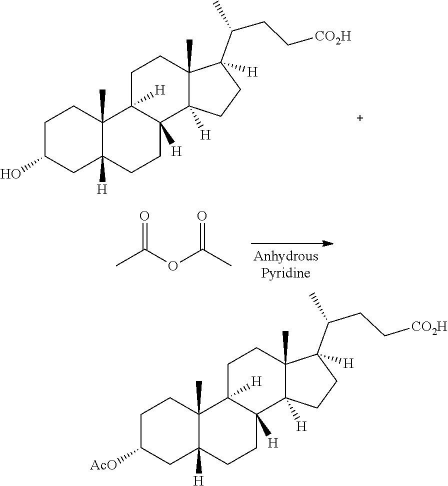

Lithocholic acid (5.4 g, 14.5 mmol) was dissolved in anhydrous pyridine (20 mL) and acetic anhydride (2.05 mL, 21.7 mmol) was added dropwise to the above solution. After stirring the reaction mixture overnight at room temperature, water (30 mL) was added and the pH was adjusted to 2 by adding 10% hydrochloric acid solution. The product was extracted with dichloromethane (50 mL×3 times). The combined organic fraction was dried over anhydrous sodium sulfate, filtered and evaporated under vacuum to give 5.8 g of white solid (95% yield).

¹H NMR (400 MHz, CDCl₃) δ: 6.6 (br, 1H); 4.70 (tt, 1H, J=5.1, J=16.5 Hz); 2.40 (ddd, 1H, J=5.3, J=10.3, J=15.7 Hz); 2.26 (ddd, 1H, J=6.4, J=9.6, J=15.9 Hz); 2.03 (s, 3H); 2.01-1.92 (m, 1H); 1.91-1.74 (m, 5H); 1.74-0.97 (m, 20H); 0.92 (d, 3H, J=6.32 Hz); 0.92 (s, 3H); 0.65 (s, 3H).

¹³C NMR (100 MHz, CDCl₃) δ: 180.0; 170.9; 74.6; 56.6; 56.1; 42.9; 42.0; 40.6; 40.3; 35.9; 35.5; 35.2; 34.7; 32.4; 31.1; 30.9; 28.3; 27.2; 26.8; 26.5; 24.3; 23.5; 21.6; 21.0; 18.4; 12.2.

### (4R)-4-[(3R,5R,8R,9S,10S,13R,14S,17R)-3-acetoxy-10,13-dimethyl-2,3,4,5,6,7,8,9,11,12,14,15,16,17-tetradecahydro-1H-cyclopenta[a]phenanthren-17-yl]pentanoyl Chloride

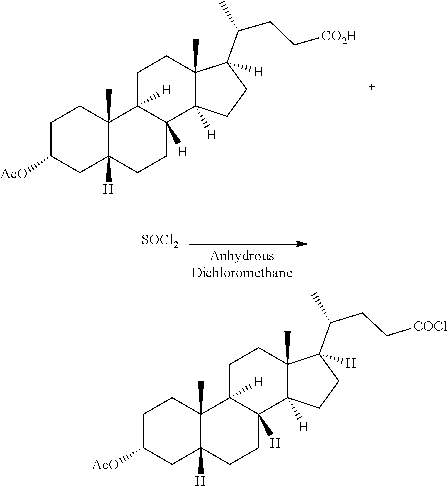

Protected lithocholic acid (10 g, 23.9 mmol) was dissolved in anhydrous dichloromethane (67 mL) and thionyl chloride (8.66 mL, 119.4 mmol) was added dropwise to the above solution. After stirring the reaction mixture at room temperature for 3 hours, the solvent and excess of thionyl chloride were evaporated under reduced pressure to give O-acetyl lithocholic acid chloride. The solid was re-suspended in anhydrous tetrahydrofuran (50 mL) before using it for the next step.

### 2,6-di-tert-butyl-4-((2-((3,5-di-tert-butyl-4-hydroxyphenyl)thio)propan-2-yl)thio)phenyl (4R)-4-[(3R,5R,8R,9S,10S,13R,14S,17R)-3-acetoxy-10,13-dimethyl-2,3,4,5,6,7,8,9,11,12,14,15,16,17-tetradecahydro-1H-cyclopenta[a]phenanthren-17-yl]pentanoate (“HA-1”)

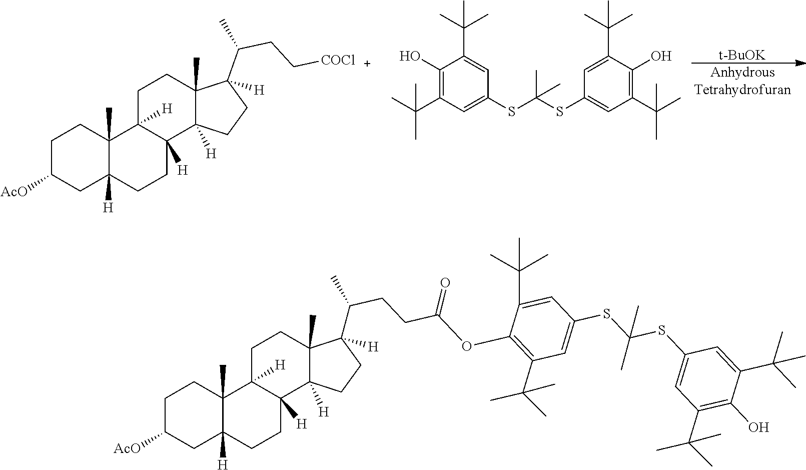

Probucol (18.5 g, 35.8 mmol) was added portion wise to a mixture of potassium tert-butoxide (8.3 g, 74.0 mmol) stirred in anhydrous tetrahydrofuran (166 mL). This mixture was left to stir at room temperature for 2 hours during which time it became dark brown. The resulting solution containing the potassium salt of Probucol was added to the suspension of protected lithocholic acid chloride. After stirring the reaction mixture at room temperature overnight, water (500 mL) was added and the product was extracted with dichloromethane (500 mL×3 times). The crude material was purified by column chromatography (silica gel, gradient hexane/dichloromethane) (51% yield, purity 93% based on ¹H NMR analysis).

¹H NMR (400 MHz, CDCl₃) δ: 7.62 (s, 2H); 7.45 (s, 2H); 5.36 (s, 1H); 4.71 (tt, 1H, J=5.0, J=16.4 Hz); 2.73-2.61 (ddd, 1H, J=5.2, J=11.2, J=16.6 Hz); 2.59-2.47 (ddd, 1H, J=6.1, J=10.5, J=16.8 Hz); 2.03 (s, 3H), 2.03-1.78 (m, 6H); 1.72-1.65 (m, 1H); 1.63-1.56 (m, 1H); 1.55 (s, 3H); 1.48-1.36 (m, 30H); 1.34 (s, 18H); 1.31-1.20 (m, 4H); 1.19-1.02 (m, 5H); 0.99 (d, 3H, J=6.4 Hz); 0.93 (s, 3H); 0.66 (s, 3H).

¹³C NMR (100 MHz, CDCl₃) δ: 173.9; 170.8; 155.2; 149.1; 142.8; 136.1 (2C); 134.8; 134.3 (2C), 129.3 (2C); 122.2 (2C); 74.6; 59.6; 56.7; 56.1; 42.9; 42.0; 40.6; 40.3; 36.0; 35.7; 35.6; 35.3; 35.2; 34.7; 34.5 (2C); 32.6; 32.4; 31.61 (3C); 31.59 (3C); 30.7; 30.4 (6C); 30.3; 28.3; 27.2; 26.8; 26.5; 24.4; 23.5; 21.6; 21.0; 18.6; 12.2.

### (4R)-4-[(3R,5R,7R,8R,9S,10S,13R,14S,17R)-3,7-diacetoxy-10,13-dimethyl-2,3,4,5,6,7,8,9,11,12,14,15,16,17-tetradecahydro-1H-cyclopenta[a]phenanthren-17-yl]pentanoic Acid

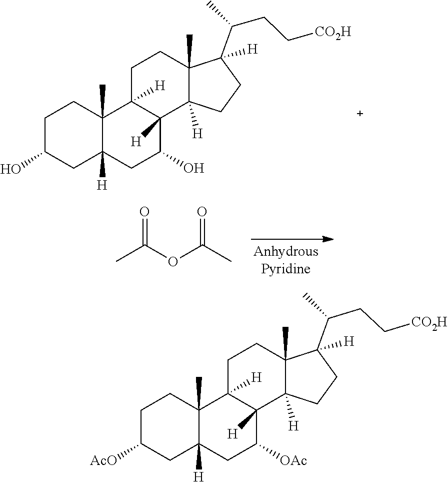

Chenodeoxycholic acid (5.0 g, 12.7 mmol) was dissolved in anhydrous pyridine (20 mL) and acetic anhydride (3.60 mL, 38.2 mmol) was added dropwise to the above solution. After stirring the reaction mixture overnight at room temperature, water (30 mL) was added and the pH was adjusted to 2 by adding 10% hydrochloric acid solution. The product was extracted with dichloromethane (50 mL×3 times). The combined organic fraction was dried over anhydrous sodium sulfate, filtered and evaporated under vacuum to give 5.5 g of white solid (91% yield).

¹H NMR (400 MHz, CDCl₃) δ: 4.92-4.83 (m, 1H); 4.59 (tt, 1H, J=4.4, J=11.4 Hz); 2.40 (ddd, 1H, J=5.1, J=10.1, J=15.4 Hz); 2.26 (ddd, 1H, J=6.5, J=9.7, J=15.9 Hz); 2.05 (s, 3H); 2.03 (s, 3H); 2.02-1.75 (m, 7H); 1.75-1.67 (m, 1H); 1.65-1.00 (m, 16H); 0.933 (d, 3H, J=6.2 Hz); 0.928 (s, 3H); 0.65 (s, 3H).

### (4R)-4-[(3R,5R,7R,8R,9S,10S,13R,14S,17R)-3,7-diacetoxy-10,13-dimethyl-2,3,4,5,6,7,8,9,11,12,14,15,16,17-tetradecahydro-1H-cyclopenta[a]phenanthren-17-yl]pentanoyl Chloride

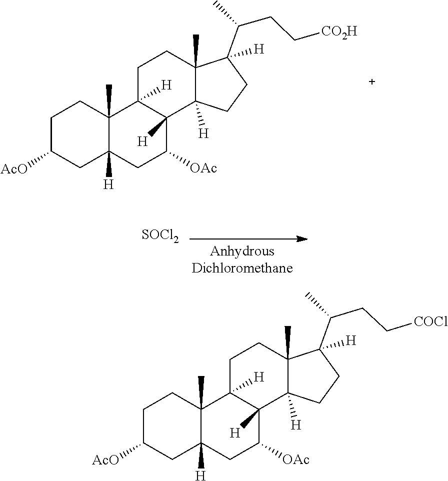

Protected chenodeoxycholic acid (1 g, 2.1 mmol) was dissolved in anhydrous dichloromethane (5 mL) and thionyl chloride (0.45 mL, 6.3 mmol) was added dropwise to the above solution. After stirring the reaction mixture at room temperature for 3 hours, the solvent and excess of thionyl chloride were evaporated under reduced pressure to give O-acetyl chenodeoxycholic acid chloride. The solid was re-suspended in anhydrous tetrahydrofuran (5 mL) before using it for the next step.

### 2,6-di-tert-butyl-4-((2-((3,5-di-tert-butyl-4-hydroxyphenyl)thio)propan-2-yl)thio)phenyl (4R)-4-[(3R,5R,7R,8R,9S,10S,13R,14S,17R)-3,7-diacetoxy-10,13-dimethyl-2,3,4,5,6,7,8,9,11,12,14,15,16,17-tetradecahydro-1H-cyclopenta[a]phenanthren-17-yl]pentanoate (“HA-2”)

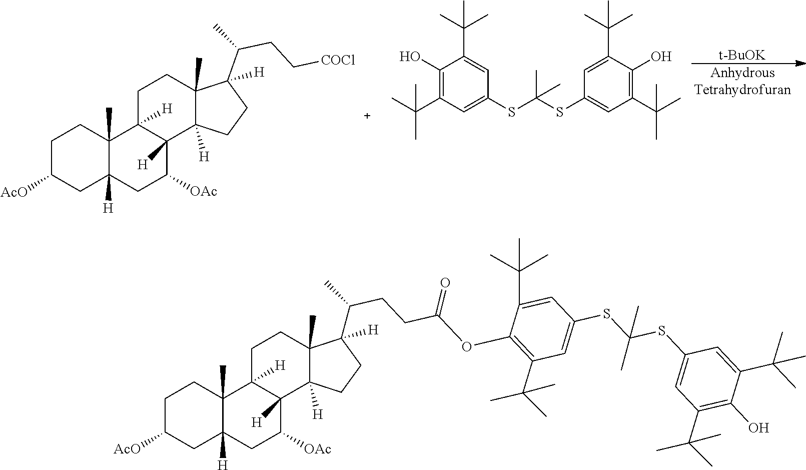

Probucol (1.6 g, 3.1 mmol) was added portionwise to a mixture of potassium tert-butoxide (0.7 g, 6.2 mmol) stirred in anhydrous tetrahydrofuran (20 mL). This mixture was left to stir at room temperature for 2 hours during which time it became dark brown. The resulting solution containing the potassium salt of Probucol was added to the suspension of protected lithocholic acid chloride. After stirring the reaction mixture at room temperature overnight, water (60 mL) was added and the product was extracted with dichloromethane (60 mL×3 times). The crude material was purified by column chromatography (silica gel, gradient hexane/dichloromethane) (37% yield).

¹H NMR (400 MHz, CDCl₃) δ: 7.62 (s, 2H); 7.45 (s, 2H); 5.36 (s, 1H); 4.92-4.85 (m, 1H); 4.59 (tt, 1H, J=4.6, J=11.5 Hz); 2.67 (ddd, 1H, J=4.9, J=10.7, J=16.0 Hz); 2.53 (ddd, 1H, J=6.1, J=10.3, J=16.6 Hz); 2.06 (s, 3H); 2.03 (s, 3H), 2.03-1.78 (m, 6H); 1.77-1.67 (m, 1H); 1.65-1.02 (m, 59H); 1.00 (d, 3H, J=6.4 Hz); 0.94 (s, 3H); 0.67 (s, 3H).

### Abbreviations

The following abbreviations are used in the Examples and Figures:
HA-1: 2,6-di-tert-butyl-4-((2-((3,5-di-tert-butyl-4-hydroxyphenyl)thio)propan-2-yl)thio)phenyl (4R)-4-[(3R,5R,8R,9S,10S,13R,14S,17R)-3-acetoxy-10,13-dimethyl-2,3,4,5,6,7,8,9,11,12,14,15,16,17-tetradecahydro-1H-cyclopenta[a]phenanthren-17-yl]pentanoate, having the formula:

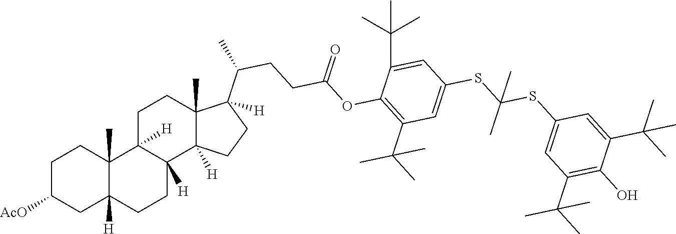

HA-2: 2,6-di-tert-butyl-4-((2-((3,5-di-tert-butyl-4-hydroxyphenyl)thio)propan-2-yl)thio)phenyl (4R)-4-[(3R,5R,7R,8R,9S,10S,13R,14S,17R)-3,7-diacetoxy-10,13-dimethyl-2,3,4,5,6,7,8,9,11,12,14,15,16,17-tetradecahydro-1H-cyclopenta[a]phenanthren-17-yl]pentanoate, having the formula:

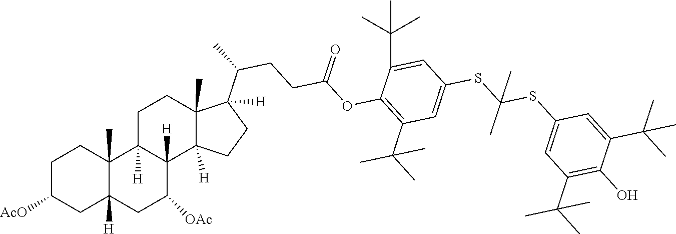

SPQ: spiroquinone (a metabolite of probucol), which is 2,4,9,11-tetrakis(1,1-dimethylethyl)-14,14-dimethyl-13,15-dithiadispiro[5.0.5.3]pentadeca-1,4,8,11-tetraene-3,10-dione, having the formula:

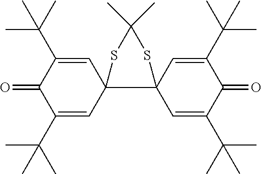 [SMILES: CC1(C)SC2(C=C(C(C)(C)C)C(=O)C(C(C)(C)C)=C2)C2(C=C(C(C)(C)C)C(=O)C(C(C)(C)C)=C2)S1]

L-Dopa: Leovdopa, having the formula:

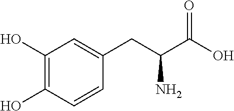 [SMILES: N[C@@H](Cc1ccc(O)c(O)c1)C(=O)O]

DMSO: dimethyl sulfoxide
SC: Sub-concussion
AlN93M: refers to a control diet (the AlN93M diet is a semi-pure maintenance diet based on a formulation developed by the American Society of Nutritional Sciences (ASNS, formerly American Institute of Nutrition)). The formulation satisfies the maintenance nutritional requirements of rats and mice. The AlN93M diet is a modification of a prior formulation AlN76A. This well studied diet was updated to separate maintenance from growing animals and improve the long term health of rodents fed semi-pure diets.
Prob: probucol
PB: probucol
AGE: aged garlic extract
ApoCIII: Apolipoprotein C—III
ApoC3: Apolipoprotein C—III
BSA bovine serum albumin
PBS phosphate buffered saline
PBST 0.1% Tween 20 in PBS

### Experimental Example 1: Effect of Probucol and its Derivatives Spq and Ha-1 on an In Vitro Model of Alzheimer's Disease

### Drug Preparation

The compounds tested were probucol, SPQ and HA-1, and a commercially available drug for the treatment of mild to moderate Alzheimer's disease (AD), rivastigmine, was used as a positive control.

Rivastigmine is a cholinesterase inhibitor and has the following structure:

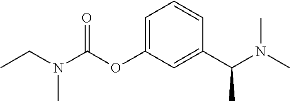 [SMILES: CCN(C)C(=O)Oc1cccc([C@H](C)N(C)C)c1]

### Stock Solutions (10 mM)

5.16 mg of probucol and SPQ, and 10.32 mg of HA-1 were dissolved in 100% DMSO to prepare 10 mM stock solutions. Rivastigmine (10 mM) was prepared in nuclease free water. For treatment, probucol (20 μM), SPQ (10 μM), HA-1 (10 μM) and rivastigmine (2 μM) were prepared from stock solution directly in Dulbecco's modified Eagle's medium (DMEM).

Perinatal tissue derived human stem cell model for Alzheimer's disease (AD) was prepared using proprietary protocols to test the effect of new drug entities. Drug treatment was for 24 h in all the assays.

### (I) Cytotoxicity Study by MTT Cell Viability Assay

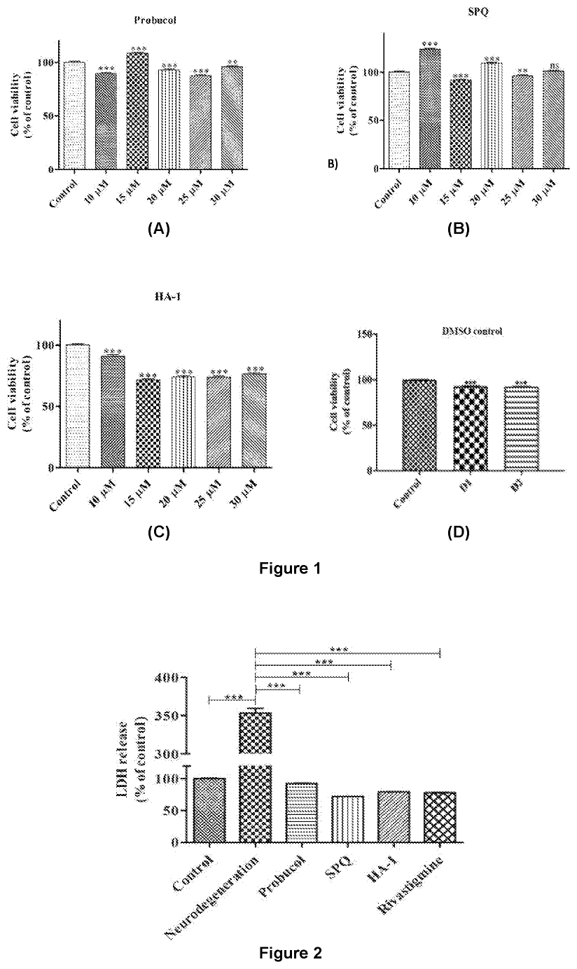

FIG. 1 shows the results of a cytotoxicity study by MTT cell viability assay. Amniotic membrane mesenchymal stem cells (AM-MSCs) were incubated with different concentrations (10, 15, 20, 25 and 30 uM, and DMSO control) of (A) probucol, (B) SPQ, (C) HA-1, and (D) DMSO for 24 hours. D1=DMSO control for 10 μM, D2=DMSO control for 20 μM (p<0.05; *p<0.05, **p<0.001, ***p<0.0001, ns=non-significant, n=3).

Probucol, SPQ, and HA-1 at the range of 10-30 μM were not toxic to the cells. For further analysis, 20 μM of probucol and 10 μM of SPQ and HA-1 was used.

### (II) Lactate Dehydrogenase (LDH) Assay

LDH leakage is a well characterised marker of cellular membrane damage and increase in indicative of lactate deficit, is a hallmark of AD. Using this model, we measured LDH levels in drug treated (PB, SPQ and HA-1 and rivastigmine) cells and compared with neurodegenerated (ND) cells.

FIG. 2 shows an estimation of LDH release. Neurodegenerated cells were incubated with probucol (20 μM), SPQ (10 μM), HA-1 (10 μM), and rivastigmine (2 μM) for 24 hours (p<0.05; *p<0.05, **p<0.001, ***p<0.0001, ns=non-significant, n=3).

It was seen clearly that probucol, SPQ, HA-1 and rivastigmine had decreased significantly LDH activity compared to ND cells, indicating reversal of AD-like neurodegeneration.

### (III) Functional Assay for Cholinergic Neurons—Acetylcholinesterase (AChE) Activity and Acetylcholine (ACh) Release

Acetylcholinesterase (AChE) that regulates hydrolysis of acetylcholine (ACh) in the brain, is an important target for treatment of Alzheimer's disease (AD), a feature of which is ACh deficiency. We assayed for AChE activity and ACh release in control, neurodegenerated AD and drug treated AD cells.

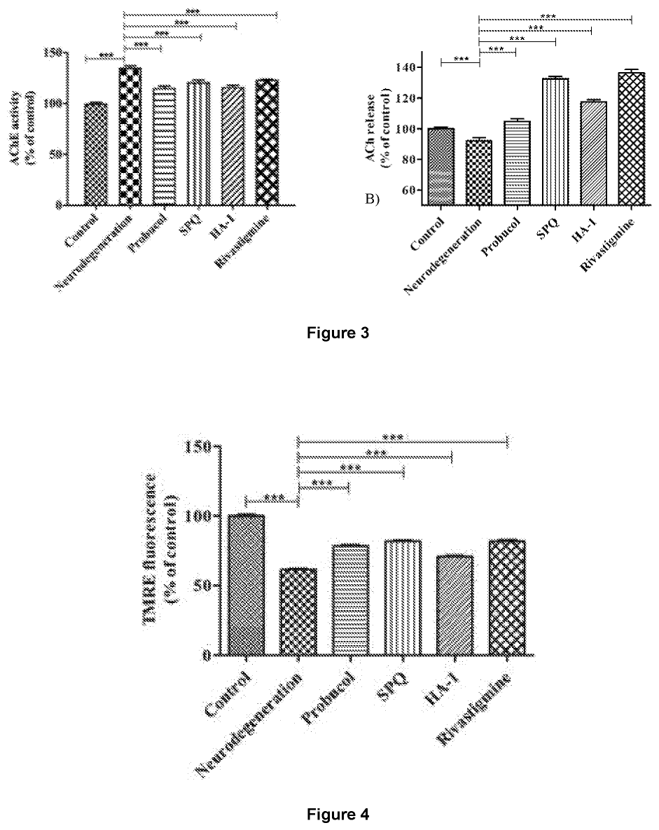

FIG. 3 shows the effect of probucol, SPQ, HA-1 and rivastigmine on (A) Acetylcholinesterase (AChE) activity and (B) Acetylcholine release in an Alzheimer's disease model using the Amplex Red reagent-based assay. Neurodegenerated cells were incubated with probucol (20 μM), SPQ (10 μM), HA-1 (10 μM) and rivastigmine (2 μM) for 24 hours (p<0.05; *p<0.05, **p<0.001, ***p<0.0001, ns=non-significant, n=3).

Inhibition of acetylcholinesterase is one of the target for treatment of AD as there is a decrease in cholinergic activity in brain of AD patients. Probucol, SPQ and HA-1 showed inhibition of acetylcholinesterase (AChE) similar to rivastigmine, an AChE inhibitor. Significantly, these compounds were also able to increase/maintain the level of acetylcholine (ACh).

### (IV) Mitochondrial Membrane Potential Test-TMRE (Tetramethylrhodamine Ethyl Ester) Assay

Aβ plaques are sources of toxicity that lead to severe structural and functional abnormalities in mitochondria in Alzheimer's disease. For assessing the changes in the electrophysiology and functional changes in the mitochondrial membrane potential, we used TMRE (tetramethylrhodamine ethyl ester) for quantifying changes in mitochondrial membrane potential in live cells by fluorimetry.

FIG. 4 shows the measurement of mitochondrial membrane potential by TMRE assay. Neurodegenerated cells were incubated with probucol (20 μM), SPQ (10 μM), HA-1 (10 μM) and rivastigmine (2 μM) for 24 hours (p<0.05; *p<0.05, **p<0.001, ***p<0.0001, ns=non-significant, n=3).

Mitochondrial dysfunction due to enhanced oxidative stress is observed in AD condition. Probucol, SPQ and HA-1 were able to maintain the mitochondrial membrane potential (similar to rivastigmine) which was disrupted due to neurodegeneration.

### (V) Gene Expression Analysis for Neuronal, Cholinergic and Ad Associated Markers

For determining the expression of genes involved in AD specific neurodegeneration and neuronal markers, we analysed BACE and chAT (AD and cholinergic markers) and synapsin 1 and neuropilin (neuronal markers).

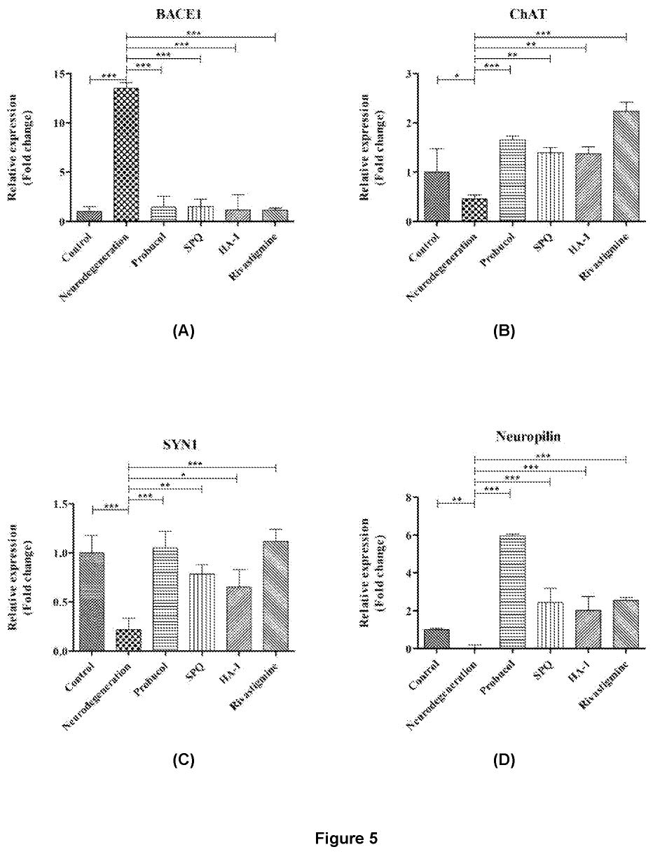

FIG. 5 shows the gene expression analysis by qRT-PCR for neuronal, cholinergic and AD associated markers (A) BACE1, (B) ChAT, (C) Synapsin 1, and (D) Neuropilin (p<0.05; *p<0.05, **p<0.001, ***p<0.0001, ns=non-significant, n=3).

Interestingly and confirming the role of amyloid beta accumulation, β-Site Amyloid Precursor Protein Cleaving Enzyme 1 (BACE1) which results in amyloid beta build-up, was significantly high in the neurodegenerated (ND)cells compared to the control cells. Further confirmation of degeneration of cholinergic neurons in the ND cells was obtained by the downregulation of choline acetyl transferase (ChAT) when compared to the control neuronal cells.

Probucol, SPQ and HA-1, similar to the positive control rivastigmine, were able to decrease the level of beta secretase 1 (BACE1), the major beta secretase required for the generation of Aβ in the neurons and increase the level of choline acetyltransferase (ChAT), which is involved in the synthesis of acetylcholine, a neurotransmitter.

Gene expression levels of mature neuronal markers, neuropilin and synapsin 1 (Syn) were also increased in neurodegenerated cells treated with probucol, SPQ and HA-1.

### (VI) Immunocytochemical Localization of Ad Specific and Neuronal Markers

For further confirmation and protein localization of the AD specific neurodegenerative markers and neuronal markers, we performed immunolocalization for BACE1 and synapsin respectively.

Fluorescence micrographs of BACE1 positive neuronal cells were obtained and the effects of probucol, SPQ and HA-1 on neurodegeneration model were compared. The accumulation of BACE1 protein is diminished in all drug treatment, with HA-1 and rivastigmine showing the least expression. In the AD model, as expected BACE1 accumulation is clearly pronounced.

Fluorescence micrographs of synapsin 1 positive neuronal cells were obtained and the effects of probucol, SPQ and HA-1 on neurodegeneration model with rivastigmine as positive control were compared. In the case of synapsin 1, a disruption in expression of synapsin protein in ND cells was observed. However, there was a clear reversal of degeneration by probucol, SPQ and HA-1.

### (VII) Estimation of Oxidative Stress: ROS (Reactive Oxygen Species) and Greiss (Nitric Oxide) Assays

Increase in oxidative stress is associated with accumulation of amyloid beta seen in the AD model. Increased oxidative stress and nitric oxide levels lead to apoptosis and evoke cytotoxic effects on neurons. We studied these parameters to check the reversal of oxidative stress in probucol, SPQ, HA-1 and rivastigmine treated AD cells.

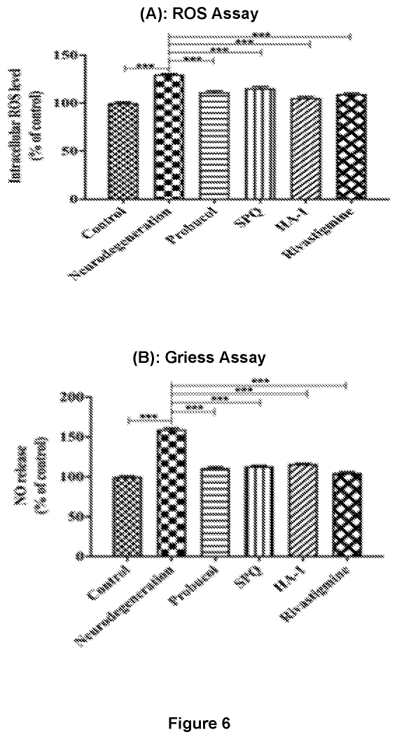

FIG. 6 shows the effect of probucol, SPQ, HA-1 and rivastigmine on (A) intracellular reactive oxidative stress and (B) nitric oxide.

Probucol, SPQ and HA-1 were able to decrease the level of ROS and prevent the reduction of endogenous nitric oxide level protecting neurons from the amyloid beta related oxidative stress.

### (VIII) Analysis of Glutathione Peroxidase Activity

Alzheimer's disease-related increase in oxidative stress is associated with decrease in the levels of brain antioxidant glutathione (GSH). We studied GSH levels to analyze if probucol, SPQ, HA-1 and rivastigmine treated AD cells had a restoration of GSH activity.

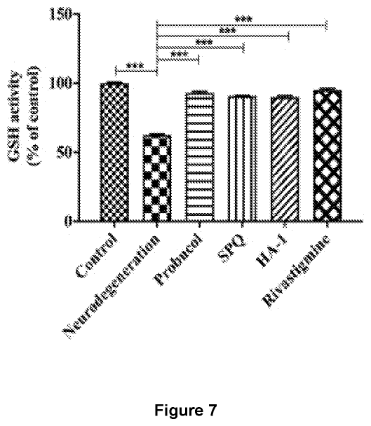

FIG. 7 shows the effect of probucol, SPQ, HA-1 and rivastigmine on glutathione activity. (Statistical analysis: results are expressed in mean±SD. One-way ANOVA followed by Dunnett's post hoc test was performed to compare all columns vs neurodegeneration (p<0.05; *p<0.05, **p<0.001, ***p<0.0001, ns=non-significant, n=3).)

The data observed correlated well and demonstrated that upon reduction of ROS and NO levels by probucol, SPQ, HA-1 and rivastigmine, as seen in FIG. 6, there was increase in the anti-oxidant GSH activity of drug treated neurons thereby indicating protection against oxidative injury (FIG. 7).

### (IX) Conclusion

Using an in vitro model of Alzheimer's disease, we observed that probucol and its derivatives SPQ and HA-1 were effective in the reversal of the specific molecular changes of AD-specific neurodegeneration. These studies prove these drugs acts as efficiently as the drug commonly used for AD, that is, rivastigmine in the AD specific parameters we tested. We observed by lactate dehydrogenase assay indicative of cellular membrane damage, mitochondrial membrane potential assay, acetyl choline levels and acetyl choline esterase activity, ROS and NO assay, glutathione peroxidase assay, that the drugs reversed the changes occurring in the neurodegenerative model. We could also demonstrate at the gene and the protein levels that the expression of the characteristic marker for AD, that is, BACE, which accumulates with amyloid beta build-up, was clearly decreased in probucol, SPQ, HA-1 and rivastigmine treated AD cells. Additionally, restoration of active neuronal markers such as synapsin and neuropilin was suggestive of a neuroprotective effect of the drugs.

### Experimental Example 2: Effect of Probucol and its Derivatives SPQ and HA-1 on an in Vitro Model of Parkinson's Disease

### Drug Preparation

The compounds tested were probucol, SPQ and HA-1, using a known drug for Parkinson's disease (PD), Levodopa (L-Dopa) as a positive control.

### Stock Solutions (10 mM)

5.16 mg of probucol and SPQ, and 10.32 mg of HA-1 were dissolved in 100% DMSO to prepare 10 mM stock solutions. L-Dopa (1 mM) was prepared in nuclease free water. For treatment, probucol (20 μM), SPQ (10 μM), HA-1 (10 μM) and L-Dopa (50 μM) were prepared from stock solution directly in Dulbecco's modified Eagle's medium (DMEM).

Perinatal tissue derived human stem cell model for Parkinson's disease (PD) was prepared using proprietary protocols to test the effect of new drug entities. Drug treatment was for 24 hours in all the assays.

### (I) Cytotoxicity Study by MTT Cell Viability Assay

An initial toxicity assay was performed.

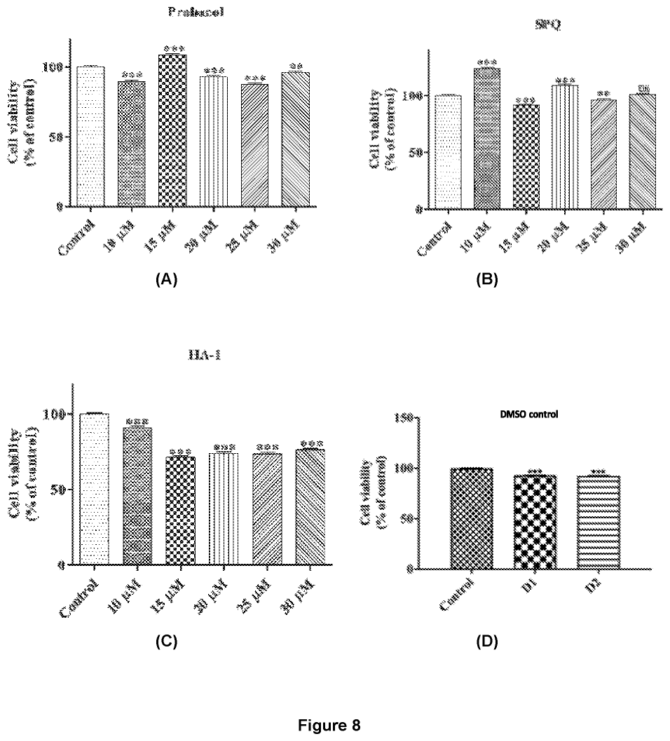

FIG. 8 shows the results of the cytotoxicity study by MTT cell viability assay. Amniotic membrane mesenchymal stem cells (AM-MSCs) were incubated with different concentrations (10, 15, 20, 25 and 30 uM, and DMSO control) of (A) probucol, (B) SPQ, (C) HA-1 and, (D) DMSO for 24 hours. D1=DMSO control for 10 μM, D2=DMSO control for 20 μM (p<0.05; *p<0.05, **p<0.001, ***p<0.0001, ns=non-significant, n=3).

Probucol, SPQ and HA-1 at the range of 10-30 μM were not toxic to the cells. For further analysis, 20 μM of probucol and 10 μM of SPQ and HA-1 was used.

### (II) Mitochondrial Membrane Potential Test-TMRE (Tetramethylrhodamine Ethyl Ester) Assay

Dysfunctional mitochondria and alterations in the mitochondrial membrane potential promote neurodegeneration seen in Parkinson's disease. For assessing the changes in mitochondrial membrane potential, we used TMRE (tetramethylrhodamine ethyl ester) to quantify the changes in live cells by fluorimetry.

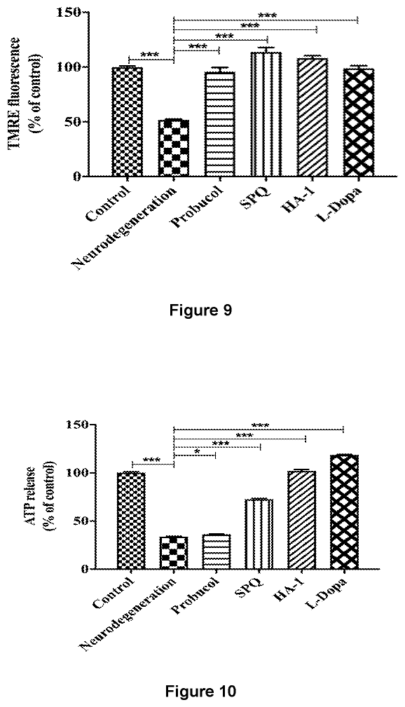

FIG. 9 shows the measurement of mitochondrial membrane potential by TMRE assay. Neurodegenerated cells were incubated with probucol (20 μM), SPQ (10 μM), HA-1 (10 μM) and L-Dopa (50 μM) for 24 hours (p<0.05; *p<0.05, **p<0.001, ***p<0.0001, ns=non-significant, n=3).

Mitochondrial dysfunction due to enhanced oxidative stress is observed in PD condition. Probucol, SPQ and HA-1 were able to maintain the mitochondrial membrane potential (similar to L-Dopa) which was disrupted due to neurodegeneration. SPQ was observed to have an enhanced restoration of the mitochondrial membrane potential.

### (III) Adenosine Triphosphate (ATP) Assay

Impaired energy metabolism and reduced ATP levels are common features of PD. Using the stem cell derived PD model, we measured ATP levels in drug treated (probucol, SPQ and HA-1 and L-Dopa) cells and compared with neurodegenerated (ND) cells.

FIG. 10 shows the estimation of ATP level. Neurodegenerated cells were incubated with probucol (20 μM), SPQ (10 μM), HA-1 (10 μM) and L-Dopa (50 μM) for 24 hours (p<0.05; *p<0.05, **p<0.001, ***p<0.0001, ns=non-significant, n=3).

It was seen clearly that PD neurodegeneration showed a significant decline of ATP levels compared to control. Enhanced increase in ATP levels was observed in SPQ and HA-1 treated cells along with L-Dopa.

### (IV) Gene Expression Analysis for Neuronal, Cholinergic and PD Associated Markers

Gene expression levels of mature neuronal marker (Synapsin I), dopaminergic markers (Nuclear receptor related 1 (Nurr1), Dopamine transporter (DAT) and Tyrosine hydroxylase (TH)), and PD associated marker (α-synuclein) were analysed to determine the effect of probucol, SPQ, HA-1 and L-Dopa.

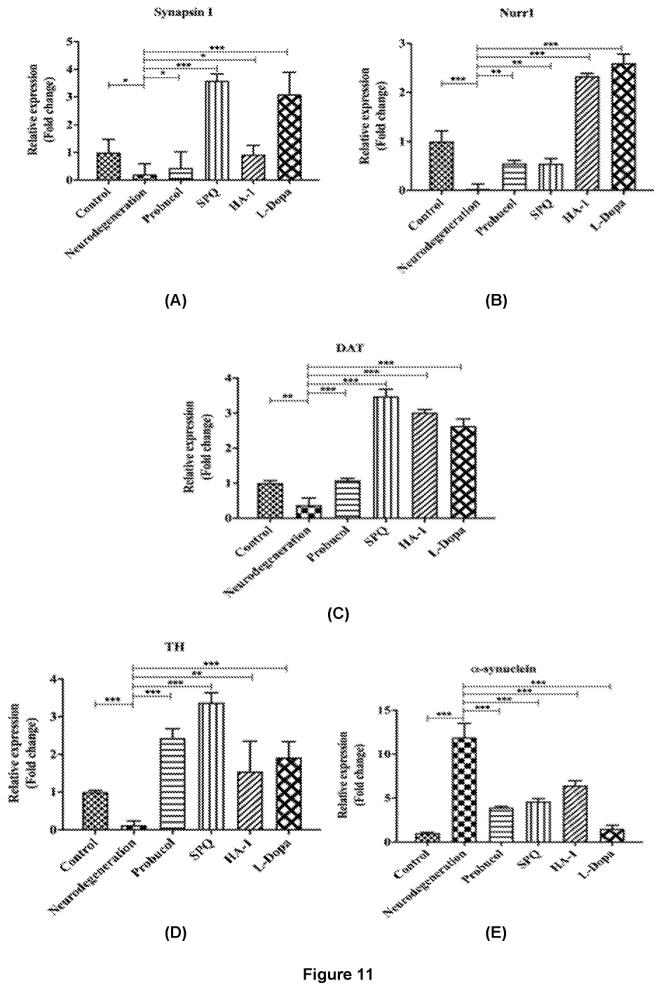

FIG. 11 shows the gene expression analysis by qRT-PCR for neuronal, dopaminergic and PD associated markers. (A) Synapsin I, (B) Nurr1, (C) DAT, (D) TH and (E) α-synuclein. (p<0.05; *p<0.05, **p<0.001, ***p<0.0001, n=3).

Probucol, SPQ and HA-1 had varying effects on the expression of Synapsin I and dopaminergic markers including Nurr1, involved in the maintenance of dopaminergic system in the brain, and DAT, which is responsible for reuptake of dopamine from the synaptic cleft. More importantly expression of TH, a rate limiting enzyme which converts L-tyrosine to L-Dopa, was as effective in drug treatment as in the positive control L-Dopa.

Comparatively, efficacy of these compounds varied as HA-1 was able to increase the expression of all the dopaminergic markers tested, whereas DAT, TH along with the neuronal marker, Synapsin I was significantly increased after SPQ treatment.

Interestingly, decreased expression of α-synuclein, accumulation of which leads Lewy body formation and ultimately degeneration of dopaminergic neurons was observed after treatments with all the three drugs probucol, SPQ and HA-1.

### (V) Immunocytochemical Localization of PD Specific and Neuronal Markers

For further confirmation and protein localization of neuronal, dopaminergic and PD specific neurodegenerative markers, we performed immunolocalization for synapsin I, Nurr1, TH and α-synuclein respectively.

Fluorescence micrographs of synapsin 1 positive neuronal cells were obtained and the effects of probucol, SPQ and HA-1 on neurodegeneration model with L-Dopa as positive control were compared. A decrease in expression of synapsin protein was observed in ND cells. However, there is a clear restoration of synapsin expression by probucol, SPQ and HA-1.

Fluorescence micrographs of Nurr1 positive dopaminergic neuronal cells were obtained and the effects of probucol, SPQ and HA-1 on neurodegeneration model with L-Dopa as positive control were compared.

Fluorescence micrographs of TH positive dopaminergic neuronal cells were obtained and the effects of probucol, SPQ and HA-1 on neurodegeneration model with L-Dopa as positive control were compared.

Appearance of the dopaminergic marker, Nurr1 and TH was seen to be more pronounced after treatment with probucol, SPQ and HA-1 after a distinct reduction in neurodegeneration.

Fluorescence micrographs of α-synuclein immunolocalization were obtained and the effects of probucol, SPQ and HA-1 on neurodegeneration model with L-Dopa as positive control were compared.

The accumulation of α-synuclein is diminished in all drug treatments, with HA-1 and L-Dopa showing the least expression. In the PD model, as expected, α-synuclein accumulation was clearly increased.

### (VI) Estimation of Oxidative Stress—ROS and Greiss (Nitric Oxide) Assays

Increase in oxidative stress associated with accumulation of α-synuclein plays a central role in disease progression. Nitric oxide enhances the aggregation of α-synuclein and accelerates disease progression. Increased oxidative stress and nitric oxide levels lead to apoptosis and evoke cytotoxic effects on neurons.

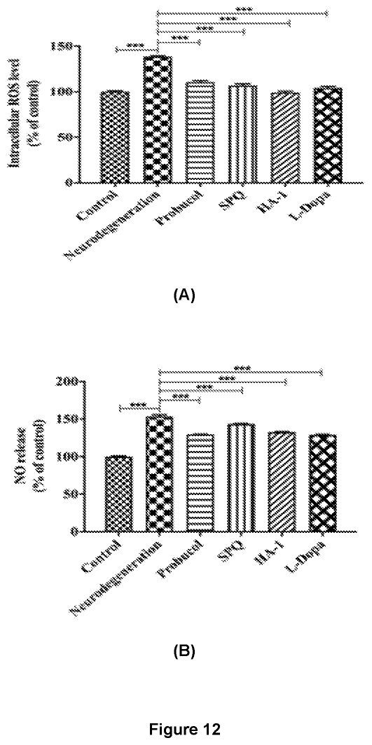

FIG. 12 shows the effect of probucol, SPQ, HA-1 and L-Dopa on (A) intracellular reactive oxidative stress and (B) nitric oxide.

Probucol, SPQ and HA-1 were able to decrease the level of ROS and prevent the reduction of endogenous nitric oxide levels thereby protecting neurons from the α-synuclein aggregation and related oxidative stress.

### (VII) Analysis of Glutathione Peroxidase Activity

Parkinson's disease-related increase in oxidative stress is associated with mitochondrial dysfunction and subsequent decrease in the levels of brain antioxidant glutathione (GSH).

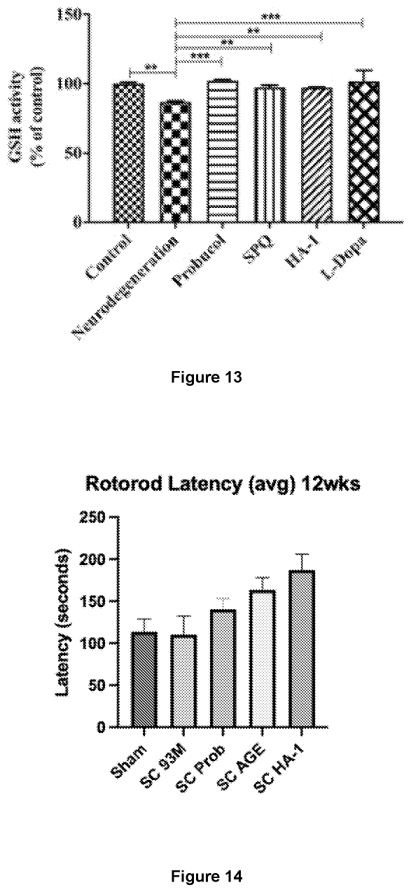

FIG. 13 shows the effect of probucol, SPQ, HA-1 and L-Dopa on glutathione activity. (Statistical analysis: Results are expressed in mean±SD. One-way ANOVA followed by Dunnett's post hoc test was performed to compare all columns vs neurodegeneration (p<0.05; *p<0.05, **p<0.001, ***p<0.0001, ns=non-significant, n=3).)

Upon reduction of ROS and NO levels by probucol, SPQ, HA-1 and L-Dopa, there was increase in the GSH activity of neurons thereby suggesting cellular and mitochondrial protection against oxidative injury (FIG. 13).

### (VIII) Conclusion

Using an in vitro stem cell derived model of Parkinson's disease, we observed that probucol, SPQ and HA-1 were effective in the reversal of the specific molecular changes of PD-specific neurodegeneration. These studies indicate that these drugs act as efficiently as the drug commonly used for PD, that is, L-Dopa in the PD specific parameters we tested. Using mitochondrial membrane potential assay, we observed mitochondrial dysfunction in PD model, which was restored by drug treatment. Furthermore, ATP assay which measures impaired energy metabolism occurring in PD, indicates clearly that the derivatives of probucol, SPQ and HA-1, were effective in reverting ATP levels. Assays such as ROS, NO and GSH also demonstrated that the drugs reversed the cellular and mitochondrial changes occurring in the neurodegenerative model.

We could also demonstrate at the gene and the protein levels that the expression of the enzyme involved in dopamine metabolism that is downregulated in PD, that is, tyrosine hydroxylase, which was suppressed in the PD model, was clearly upregulated in probucol, SPQ, HA-1 and L-Dopa treated PD cells. Significantly, the drugs also substantially reduced the accumulation of α-synuclein, the major factor responsible for accelerating neuronal degeneration. Additionally, restoration of active neuronal markers such as synapsin and dopaminergic markers, Nurr1, DAT and TH was indicative of a neuro-regenerative effect of the drugs.

### Experimental Example 3: Brain Injury (Sub-Concussion) Study

Repeated sub-concussive head impacts are reported to impair psychomotor function. Rats were subjected to sham procedure, sub-concussion (SC) with control diet (AlN93M), SC with probucol, SC with aged garlic extract (AGE), or SC with HA-1 for 12 weeks.

Both rotarod and beam walk tests assess neuromotor function. On beam walk, the higher the slips show the more impairment. On rotarod, the lower the latency indicates the more impairment.

### Rotarod Test

FIG. 14 shows rotarod latency (average) in seconds at 12 weeks after sham procedure, sub-concussion with administration of AlN93M, sub-concussion with administration of probucol, sub-concussion with administration of AGE, and sub-concussion with administration of HA-1.

Rats with SC did not show any changes in the latency indicating no detectable neuromotor deficits. Nonetheless, the treatment with HA-1 significantly increased the latency, indicating higher neuromotor performance. Thus, the rats on HA-1 performed the best on rotarod as shown by the higher latency compared with sham, SC with AlN93M, SC with probucol, and SC with AGE.

### Beam Walk Test

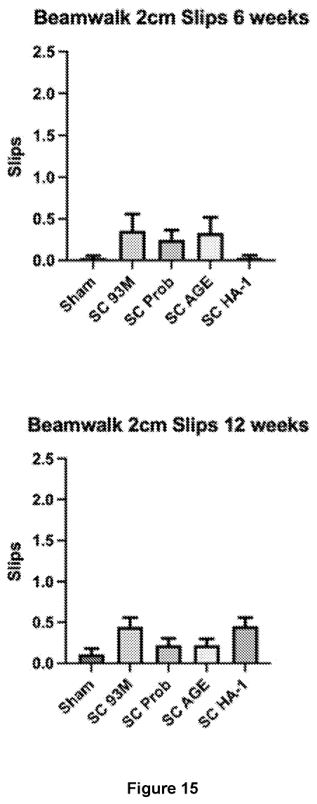

FIG. 15 shows the number of slips (Y axis) in a beam walk test (2 cm) at 6 weeks and 12 weeks after sham procedure, sub-concussion with administration of AlN93M, sub-concussion with administration of probucol, sub-concussion with administration of AGE, and sub-concussion with administration of HA-1.

After 6 weeks of experiment/drug intervention, SC increased beam walk slips on 2 cm beam. Whilst probucol or AGE did not indicate any beneficial effects, HA-1 showed significant reduction in slips on 2 cm beam. Thus, HA-1 showed significant prevention of neuromotor dysfunction particularly on 2 cm beam walk after 6 weeks, as shown by the lower number of slips compared with SC with AlN93M, SC with probucol, and SC with AGE.

At 12 weeks, on all 1, 2 and 3 cm beam walk, SC increased the number of slips. The provision probucol, AGE or HA-1 did not show significant preventative effects in reducing slips. No effects were seen after 12 weeks.

### Experimental Example 4: Viability Assays of HA-1 on Pancreatic NIT-1 Cells

### (I) Culturing Pancreatic NIT-1 β-Cells

Cells were grown and processed in a Dulbecco's Modified Eagle's media with 11.5% foetal bovine serum, as per the protocols described below.

Specifically, the cells were cultured on T-75 cm² tissue culture flasks (Thermo Fisher Scientific®, Australia) and fed with Dulbecco's modified Eagle's medium (DMEM) (Gibco, Life Technologies, USA) supplemented with either 5.5 mM glucose or 25.5 mM glucose (Sigma Chemical Co, USA), 11.5% fetal bovine serum (Thermo Fisher Scientific, Australia), and 5% penicillin-streptomycin (Thermo Fisher Scientific, Australia). The β-cells were incubated in an environment of 5% CO₂ in humidified air at 37° C. using a Nuaire NU-8500 Water Jacket CO₂ Incubator (Nuaire, USA) for 72 hours.

### (II) MTT Viability Protocols

The MTT assay for cellular mitochondrial activity is a common and an important analytical technique for determining the degree of cell viability and biological activity. Briefly, MTT was prepared as 5 mg/ml stock solution (Sigma Chemical CO, USA) in phosphate buffer, at pH 7.4 (Thermo Fisher Scientific, Australia). The undissolved residues were removed by sterile filtration. The stock solution was stored in a sterile environment at 4° C. in the dark and used within 7 days of preparation. For the MTT assay protocol, 20 μl of MTT from the stock solution were added into each well of 96-well plates (Thermo Fisher Scientific, Australia) containing 10 μM probucol and 10 μM HA-1 (dissolved in propylene glycol-water hydrogel mixture). The MTT conversion to formazan was removed from the incubator after 4 hours by washing the microcapsules with MilliQ water for 5 minutes in order to remove spectroscopic interference. Formazan was dissolved in 100 μl of dimethyl sulfoxide (DMSO) (Sigma Chemical CO, USA) via reverse pipetting and the resultant purple solution was analysed photometrically at 550 nm.

### (III) Results and Discussion

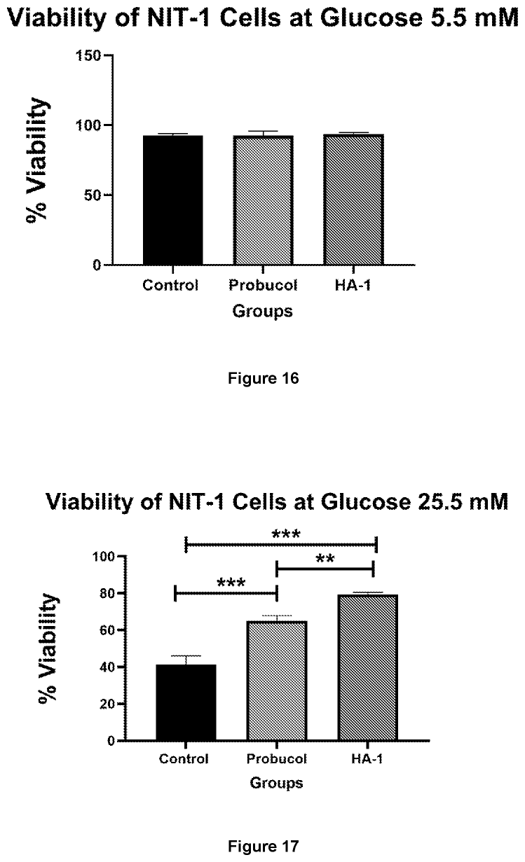

FIG. 16 shows that under euglycemic conditions, none of the treatment groups exerted any effects and this was expected as under physiological conditions NIT-1 cells are already at optimal functionality.

However, at hyperglycemic states, NIT-1 cells are under stress due to glucose-mediated toxicity, mitochondrial dysfunction and oxidative stress (FIG. 17). Probucol was able to partially offset hyperglycaemia-induced pancreatic beta cell death but HA-1 had an even more profound effect on ameliorating cell death and being able to substantially offset beta-cell damage from glucose toxicity.

### (IV) Conclusion

HA-1 confers potent pancreatic beta-cell protective properties, ensuring cellular viability and functionality under hyperglycemic states. This has great ramifications for diabetes mellitus therapy, and HA-1 will be of notable benefit in the context of diabetes mellitus management.

### Experimental Example 5: Cognitive Function (Memory) Test

Cognitive function in diabetic db/db mice was tested with Passive Avoidance and Novel Object Recognition tests. For both tests, the higher the value, the better the memory.

The mice were administered with either probucol, HA-1 or apoCIII antisense from 5 weeks of age, and cognitive performance was tested at 14 weeks of age.

HA-1 showed to completely restore the memory in diabetic mice, equivalent to probucol or apoCIII antisense.

### (I) Passive Avoidance Test

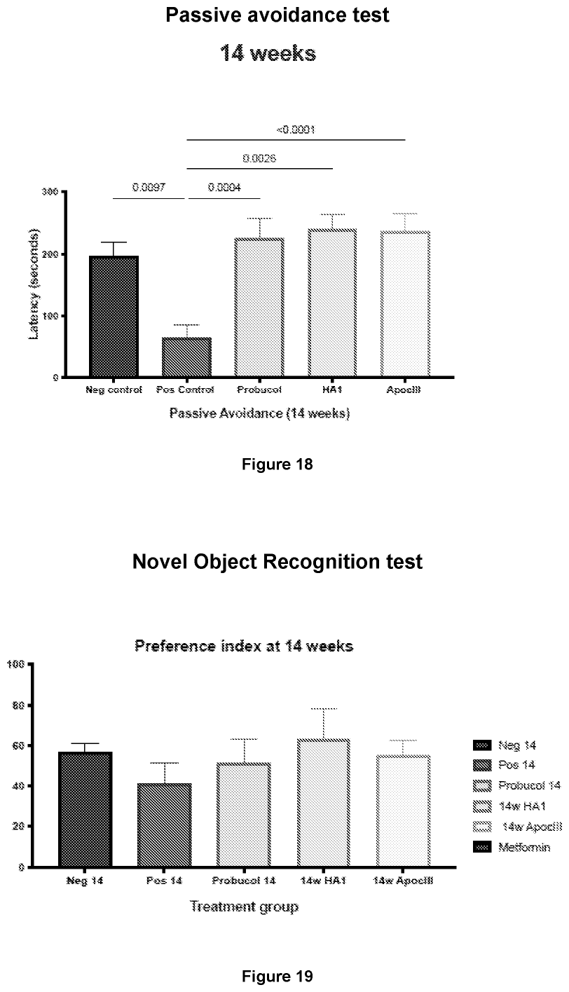

FIG. 18 shows the results of the Passive Avoidance Test at 14 weeks for negative control (non-diabetic mice), positive control (diabetic db/db mice), probucol, HA-1 and ApocIII.

At 14 weeks, diabetic db/db (Pos Control) mice showed significantly reduced latency compared to non-diabetic control mice (Neg control), indicating marked short-term memory loss. Provision of probucol completely prevented the memory loss, showing equivalent latency to the Neg control mice. Similarly, mice that were given HA-1 showed equivalent latency to Neg control and probucol group mice. Mice that were maintained on apoCIII antisense (ApoCIII) also showed preventative effects.

### (II) Novel Object Recognition Test

FIG. 19 shows the results of the Novel Object Recognition Test at 14 weeks for negative control (non-diabetic mice), positive control (diabetic db/db mice), probucol, HA-1 and ApocIII.

At 14 weeks of age, db/db pos mice did not show any memory deficits, indicating comparable preference index to negative control mice. Treatment with probucol, HA-1 or ApocIII showed no significant effects at 14 weeks of age.

### Experimental Example 6: Effects of HA-1 on Repeated Sub-Concussion in Rats

Repeated sub-concussive head impacts are increasingly recognised to cause damage to the brain and neuromotor dysfunction. This study investigated the effects of HA-1 on preventing the disruption of the blood-brain barrier (BBB) and on attenuating neuroinflammation and neuromotor dysfunction in an established rat model of repeated sub-concussion.

Female PVG rats aged 5-7 weeks were used. Under complete anaesthesia with isoflurane, a 25 g weight was dropped onto lambda of the skull from a 1 m height. This was repeated consecutively for 10 times per procedure. The procedure was repeated 3 times per week (i.e., Monday, Wednesday, Friday) for the duration of 12 weeks. Sham group rats received the same procedure without the weight drops. The probucol treatment group received probucol which was supplemented into the diet at 0.1% w/w. The HA-1 treatment group received HA-1 also in the diet at 0.2% w/w for the entire duration of the study.

After the completion of 12-week sub-concussion procedures, neuromotor function was tested with established rotarod and beamwalk tests. On beamwalk, the higher the slips show, the more impairment. On rotarod, the lower the latency indicates the more impairment.

The rats were then sacrificed to obtain brain tissue for further analyses.

### Rotarod Test

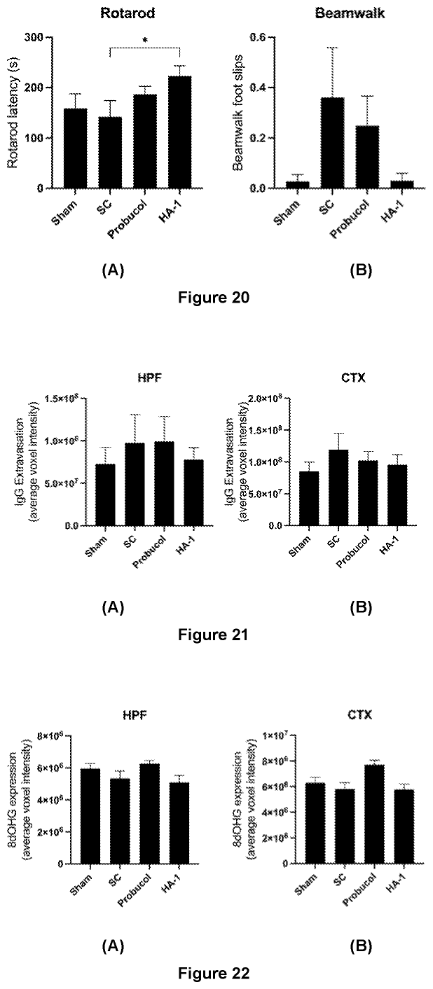

The rotarod test (FIG. 20A) showed no significant changes in the latency of rats that received repeated sub-concussion (SC) impacts, compared to the sham rats, indicating no substantial neuromotor deficits (*p<0.05, n=12).

Supplementation with probucol also did not show any changes. However, HA-1 treatment indicated significant increase in the latency, indicating improved neuromotor performance.

### Beamwalk Test

The beamwalk test (FIG. 20B) indicated substantial increase in foot slips in SC rats compared to sham, suggesting marked neuromotor dysfunction. This was modestly attenuated by the provision of probucol, whilst HA-1 completely normalised it to a level comparable to the sham group, indicating that treatment with HA-1 improved neuromotor performance.

### Blood-Brain Barrier (BBB) Integrity

Following the 12-week repeated sub-concussion, the rats showed increase in IgG extravasation in hippocampal formation (HPF) and cortex (CTX) regions of the brain, compared to rats that received sham procedure (FIGS. 21A and 21B). The increase of IgG extravasation in SC rats indicates disruption of the BBB, allowing blood-borne IgG to leak into the brain. Probucol supplementation showed no protection against BBB disruption in HPF, while there was some reduction in the CTX IgG extravasation. HA-1 was found to be more effective than probucol in attenuating BBB disruption in both HPF and CTX regions.

### Neuronal Oxidative Stress

Repeated sub-concussive impacts did not alter the expression of 8dOHG, a marker of oxidative stress (FIGS. 22A and 22B). Interestingly, probucol showed some increase of 8dOHG particularly in the CTX. However, the administration of HA-1 substantially attenuated the cerebral oxidative stress levels, even lower than the sham rats.

### Conclusion

The data collectively indicate that HA-1 prevents the disruption of the BBB in rats receiving SC head impacts, leading to the attenuation of oxidative stress. These effects were better than the effects observed from probucol. Thus, administration of HA-1 results in the improvement of neuromotor performance, preventing the dysfunction induced by repeated SC.

### Experimental Example 7: Effects of HA-1 on Diabetes-Induced Cerebral Blood-Brain Barrier Disruption and Cognitive Decline

It is well-established that diabetes increases the risk of developing dementia, in particular, Alzheimer's disease. Although the exact underlying mechanisms are unknown, an emerging body of evidence suggests that the disruption of the BBB may be pivotal. Thus, we examined in a clinically relevant mouse model of type 2 diabetes, diabetic db/db mice, the effects of HA-1 in comparison to probucol on BBB integrity and dementia phenotypes.

In this study, 4-5 week old diabetic db/db mice were put onto a diet containing probucol 0.1% (w/w) or HA-1 0.2% (w/w) for either 10 or 24 weeks, till the age of 14 or 28 weeks, respectively. Passive avoidance test was used to assess short-term memory. Subsequently, the mice were sacrificed to obtain plasma and brain tissue samples for further analyses.

### Diabetic Phenotype

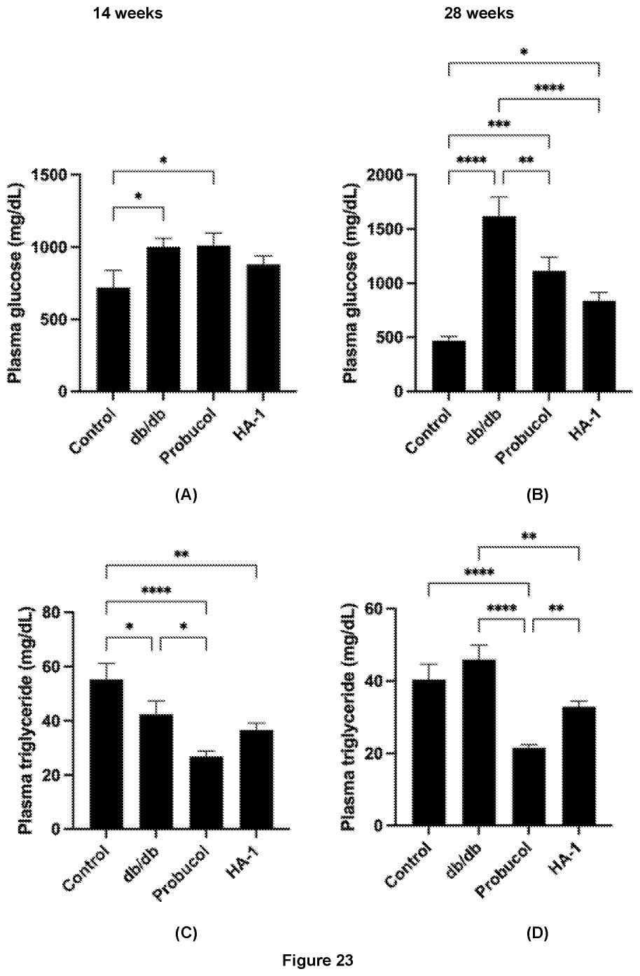

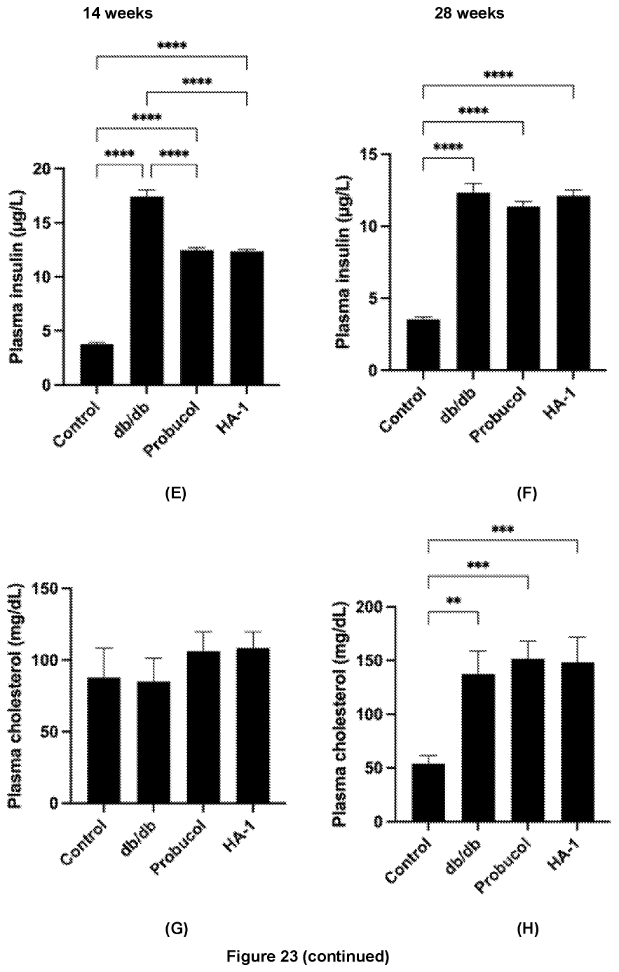

Elevation in plasma glucose, triglycerides and insulin are typical signs of diabetes, with cholesterol to lesser extent. Levels of plasma glucose, plasma triglyceride, plasma insulin, and plasma cholesterol levels were measured at 14 weeks of age and at 28 weeks of age (FIG. 23) (*p<0.05, **p<0.01, ***p<0.001, ****p<0.0001, n=12).

At 14 weeks of age, diabetic db/db mice had significantly higher glucose compared to non-diabetic control mice (FIG. 23A). Probucol showed no significant effects, and HA-1 showed modest decrease in blood glucose of db/db mice. At 28 weeks of age, probucol and HA-1 significantly reduced blood glucose with HA-1 presenting greater effects (FIG. 23B).

In 14-week old diabetic db/db mice, plasma triglyceride levels did not increase compared to control mice (FIG. 23C). Nonetheless, probucol and HA-1 significantly attenuated plasma triglycerides at 14 weeks of age (FIG. 23C). Similarly, at 28 weeks of age, probucol and HA-1 significantly reduced plasma triglycerides (FIG. 23D).

Plasma insulin levels were increased in diabetic db/db mice at 14 weeks of age (FIG. 23E). The insulin levels were significantly reduced by the provision of probucol or HA-1 at 14 weeks of age (FIG. 23E), although the insulin levels remained significantly higher than the non-diabetic control mice. At 28 weeks of age, probucol or HA-1 lost the ameliorative effects on insulin. Similarly, probucol and HA-1 showed no beneficial effects on plasma cholesterol (FIGS. 23G and 23H).

### Alzheimer's Amyloid-β

An emerging body evidence suggests that amyloid-beta (Aβ) in circulation is associated with the risk of Alzheimer's disease and cognitive decline. Particularly, Aβ42 appears to be more toxic whilst soluble Aβ40 is considered less harmful. An increasing number of studies also show that the plasma concentration of Aβ oligomers and the Aβ42/40 ratio are also highly associated with Alzheimer's disease and cognitive decline.

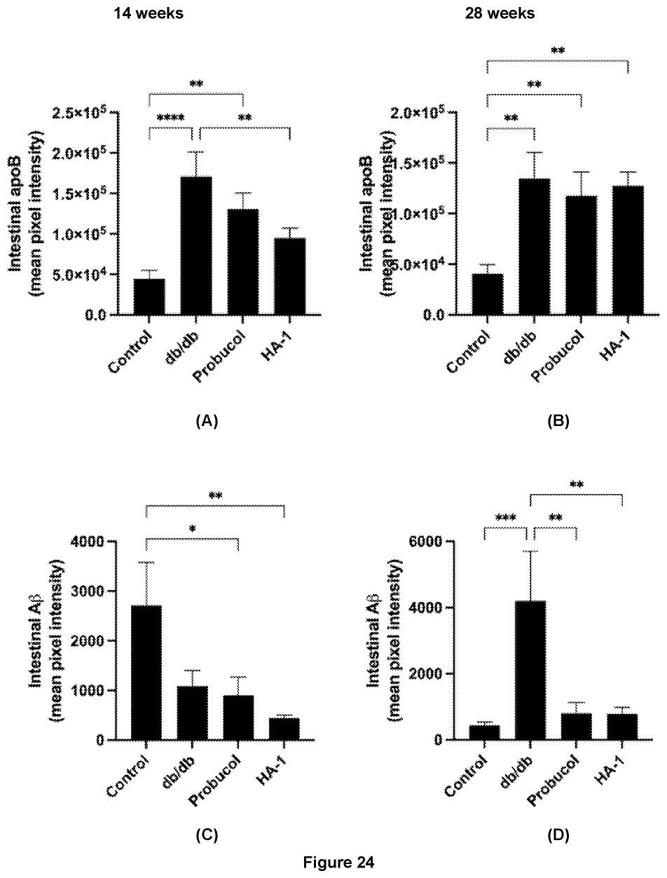

As expected, diabetic db/db mice showed significantly higher levels of apoB (a marker of fat carrying particles) in the small intestine at 14 and 28 weeks of age (FIGS. 24A and 24B), consistent with the increase in plasma triglycerides and cholesterol. The increase in small intestinal apoB was significantly attenuated by HA-1 at 14 weeks of age, whereas probucol showed no significant reduction and apoB remained significantly higher than the control mice (FIG. 24A) (*p<0.05, **p<0.01, ***p<0.001, ****p<0.0001, n=12). The ameliorative effects of HA-1 on intestinal apoB were no longer seen in 28-week-old diabetic db/db mice (FIG. 24B).

Small intestinal Aβ (FIGS. 24C and 24D) was not increased in the 14-week-old diabetic db/db mice, while it was significantly increased in 28 week db/db mice. Probucol significantly attenuated the small intestinal production of Aβ in db/db mice at 14 and 28 weeks of age. HA-1 showed comparable or superior effects in attenuating small intestinal Aβ (FIGS. 24C and 24D).

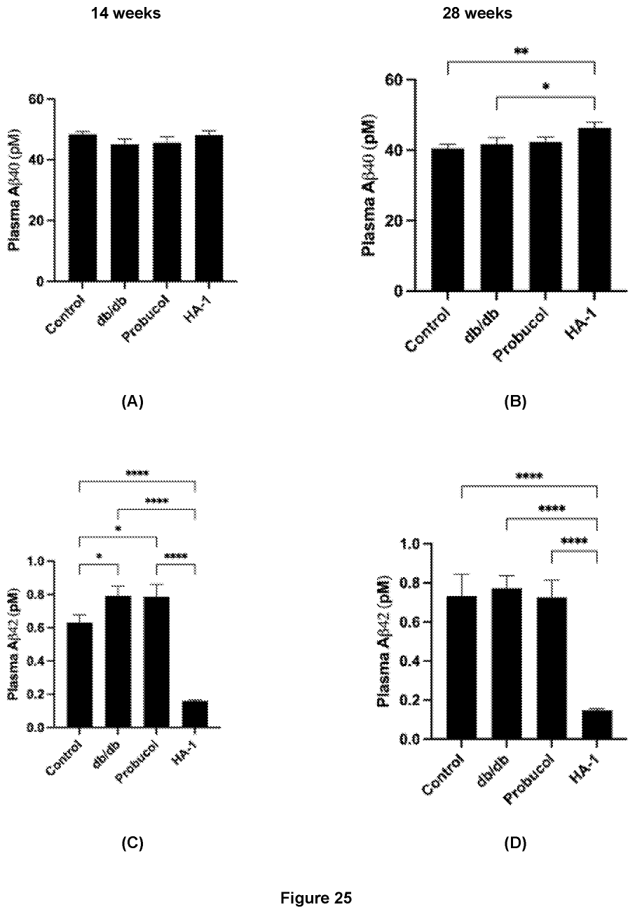

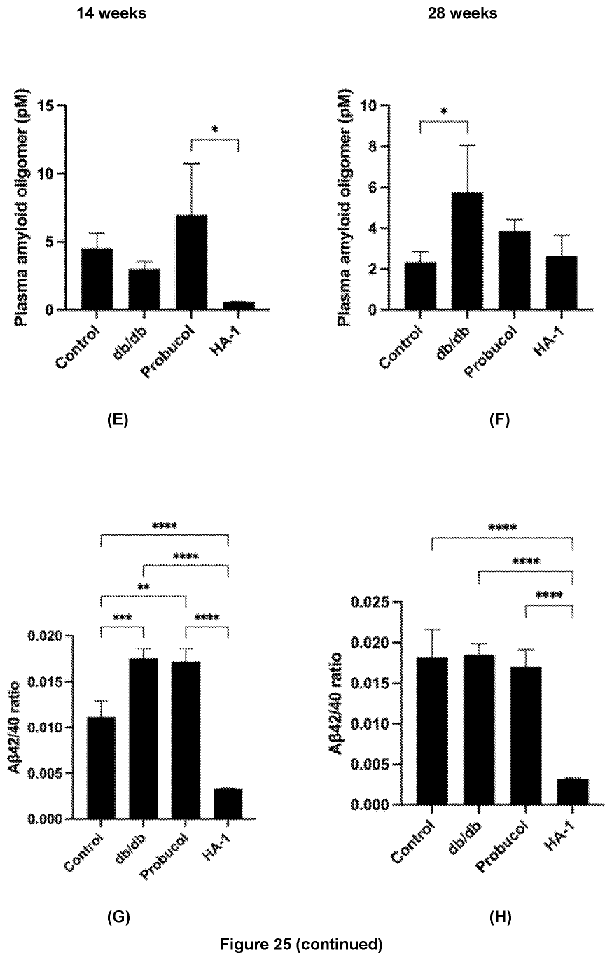

In plasma, diabetes did not increase the non-toxic form of amyloid, Aβ40 (FIGS. 25A and 25B) (*p<0.05, **p<0.01, ***p<0.001, ****p<0.0001, n=12). Probucol and HA-1 did not change plasma Aβ40 levels in 14-week old diabetic db/db mice (FIG. 25A), whilst HA-1 increased plasma Aβ40 levels in 28-week old diabetic db/db mice (FIG. 25B).

At 14 weeks of age, plasma levels of toxic Aβ42 was significantly increased in diabetic db/db mice compared to control mice (FIG. 25C). This was remarkably attenuated by HA-1, whilst probucol indicated no beneficial effect (FIG. 25C). Similarly at 28 weeks, although diabetic db/db mice did not show increase in plasma Aβ42, only HA-1 significantly reduced it (FIG. 25D).

Plasma concentrations of Aβ oligomers (FIGS. 25E and 25F) were also significantly reduced with HA-1 at 14 weeks of age and 28 weeks of age, which was not observed with probucol. Similarly, the Aβ42/40 ratio was remarkably reduced with HA-1 in db/db mice at 14 weeks of age and 28 weeks of age (FIGS. 25G and 25H).

Collectively, these data suggest that HA-1 may significantly reduce the risk of Alzheimer's dementia (dementia caused by Alzheimer's disease) via ameliorating diabetes-induced dyslipidaemia and modulating plasma Aβ levels.

### Memory Function

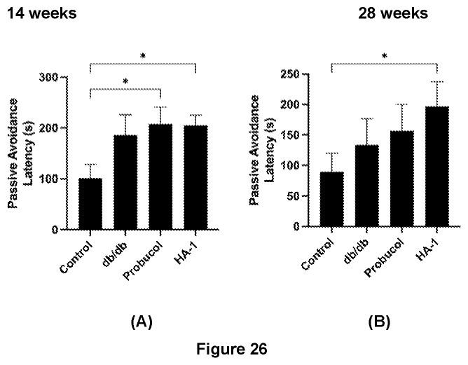

Consistent with our observations in plasma biomarkers, HA-1 appeared to significantly improve short-term memory in db/db mice using passive avoidance test (FIGS. 26A and 26B) (*p<0.05, n=12). Interestingly, db/db mice showed higher latency in the test compared to control mice, indicating no notable memory deficits at 14 or 28 weeks of age. Nonetheless, compared to the control mice, HA-1 significantly increased the latency, indicating improved short-term memory.

### BBB Integrity

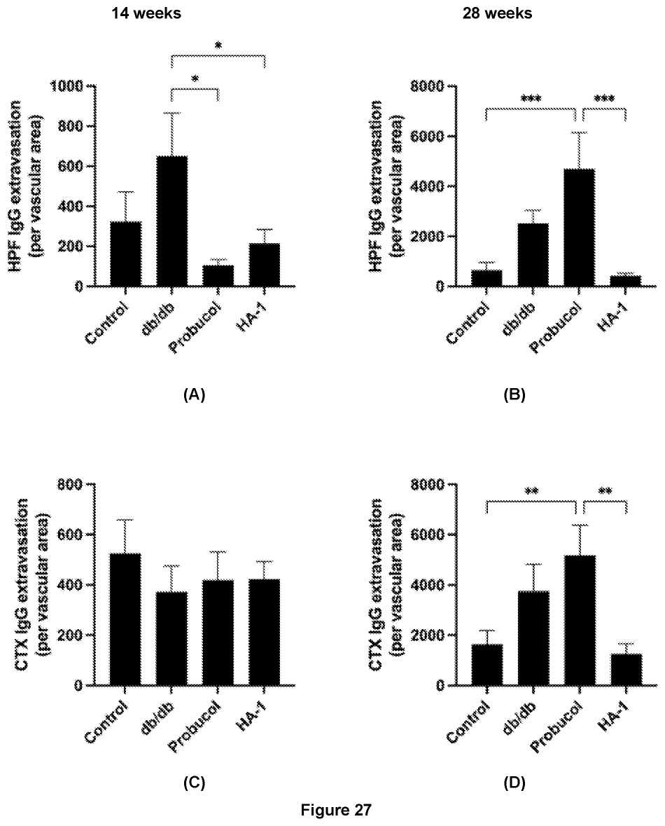

Diabetic db/db mice showed substantially higher brain leakage of IgG in the hippocampal formation (HPF) and cortex (CTX) (FIGS. 27A, 27B and 27D), indicating the disruption of the BBB, except for the CTX at 14 weeks of age (FIG. 27C) (*p<0.05, **p<0.01, ***p<0.001, n=12).

At 14 weeks of age, HPF levels of IgG extravasation were significantly attenuated by the provision of probucol or HA-1 (FIG. 27A), completely preventing the disruption of the BBB. In contrast, probucol did not show BBB protection in HPF at 28 weeks (FIG. 27B), whilst HA-1 significantly attenuated the BBB breakdown (FIG. 27B). Similarly in 28-week old db/db mice, probucol showed no beneficial effects on CTX IgG extravasation, but HA-1 significantly reduced this (FIG. 27D).

### Oxidative Stress and Neuroinflammation

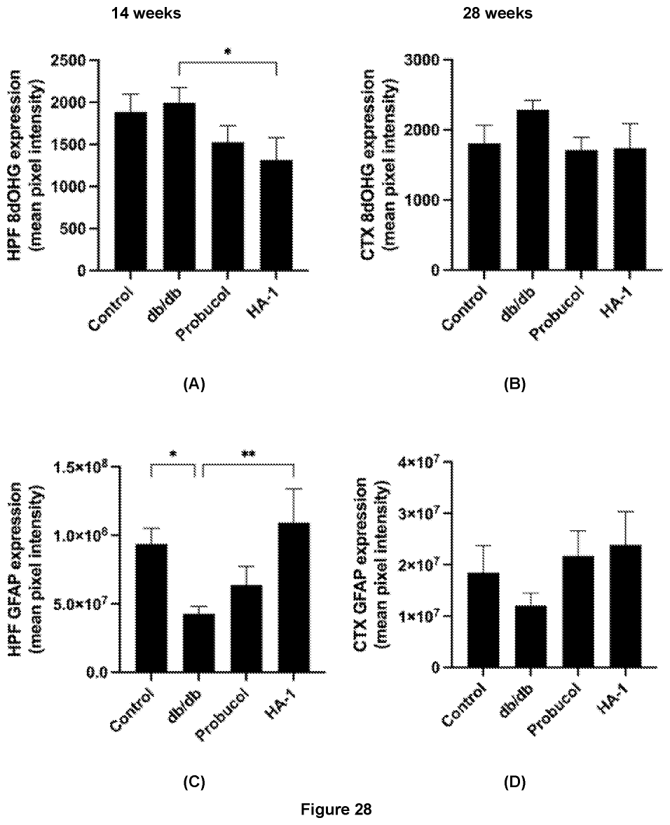

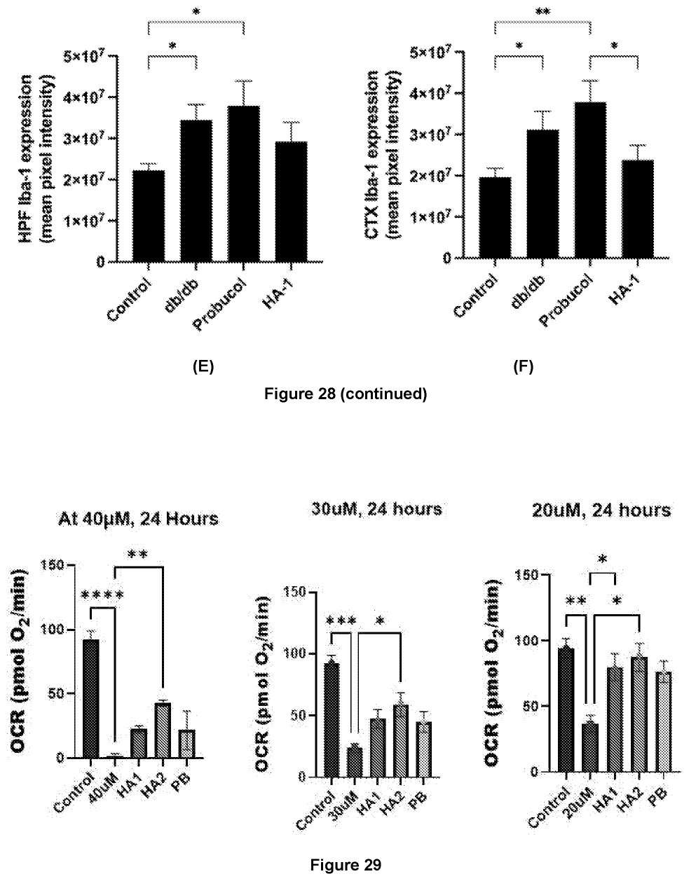

Diabetic db/db mice showed some increase in the HPF and CTX expression of 8dOHG (FIGS. 28A and 28B), indicating increased oxidative stress. This was attenuated by both probucol and HA-1 (FIGS. 28A and 28B), but HA-1 showed better efficacy particularly in HPF (FIG. 28A) (*p<0.05, **p<0.01, n=12). GFAP is a marker of astrocyte activation, which may confer anti-inflammatory effect. GFAP was significantly reduced in HPF of db/db mice (FIG. 28C), which showed significant recovery by the provision of HA-1, although probucol showed no benefits. In CTX, GFAP was moderately reduced (FIG. 28D), which was ameliorated similarly by probucol and HA-1.

Iba-1 expression indicates microglial activation, which is a surrogate marker of neuroinflammation. Iba-1 was significantly higher in the HPF and CTX in diabetic db/db mice (FIGS. 28E and 28F). HA-1 substantially reduced the expression of Iba-1 in the HPF and CTX (FIGS. 28E and 28F). In contrast, probucol showed no beneficial effects and Iba-1 expression remained significantly elevated (FIGS. 28E and 28F).

### Conclusion

The data collectively indicate that HA-1 improves diabetic phenotype of clinically relevant mouse model of diabetes, db/db mice. HA-1 also significantly reduced the systemic level of Aβ, particularly the toxic Aβ42. The latter resulted in the prevention of BBB disruption, significantly attenuating the oxidative stress and neuroinflammation. These collectively led to the prevention of memory deficits. The data indicate that HA-1 may be effective in preventing or treating Alzheimer's disease via various aspects.

### Experimental Example 8: Effects of HA-1 and HA-2 on Otoprotection

Several experiments were performed to evaluate the potential otoprotective effects of two analogues, HA-1 and HA-2.

### Materials and Methods (all Data Based on OC-1 Cells)

### Seahorse Experiment

Mitochondrial function was analysed by measuring oxygen consumption rate (OCR) using real-time Seahorse Flux Analyser XF 96 (Seahorse Bioscience, USA) with our well-established method (Kovacevic, Bozica, et al. “Novel hydrogel comprising non-ionic copolymer with various concentrations of pharmacologically active bile acids for cellular injectable gel.” Colloids and Surfaces B: Biointerfaces (2022): 113014). Briefly, the first injection was with media without glucose, with subsequent ATP synthase inhibitor oligomycin and FCCP (carbonyl cyanide-p-trifluoromethoxy phenylhydrazone) and the final injection was complex I+II inhibitors Rotenone+antimycin A 77. The result was automatically generated and analysed by the wave software. The OC-1 cells were pre-treated with cisplatin at three different concentrations (20, 30 and 40 μM) for 24 or 48 hours to induce cytotoxicity prior to the seahorse bioenergetics assays.

### Caspase Assay

The caspase assay was carried as per manufacturer's instructions (abcam, NSW, Australia), as described below.

### Analysis of Caspase 3/9 Protein:

### Material Required

Collagen coated cover slip
Fixing agent: 4% paraformaldehyde
Permeabilization agent 0.5% of Tween 20 or trition-X 100 diluted in PBS
Blocking: 1% BSA in PBST may also be used.
PBST: 0.1% Tween 20 in PBS
Primary abs (1:500 in PBS)
Secondary abs (1:1000 in PBS)
Hoechst (Nuclear stain)

### Procedure:

First day: Fixation, permeabilize, blocking and add a primary antibody to the cells, as described below:

### A) Fix the Cells

Start by aspirating culture medium from each well followed by fixation of the cells with 4% Using 4% paraformaldehyde in PBS pH 7.4 for 10 min at room temperature. (Add one ml in each well).
Shake a plate for a few seconds.

### b) Cellular Permeabilization

0.5% of Tween 20 or trition-X 100 diluted in PBS. Tween-20 is used for epitopes located in cytoplasm while triton-x100 is used for permeabilizing the nucleus and mitochondria.
1) Incubate wells with permeabilization buffer for 10 minutes at room temperature.
2) Aspirate permeabilization buffer and wash PBS for 5 minutes each time.

### Blocking:

block unspecific binding sites with blocking buffer for 1 hour at room temperature.
In this one hour, prepare primary abs by diluting it in blocking buffer.
Wash 3 times with PBST for 5 minutes.

### Primary Abs: (1: 500)

Treat the cells with 1 abs and incubate cells at 4 degree overnight.
Aspirate off primary abs solution following by washing cells three times with PBST for five minutes each.
Treat cells with secondary abs for an hour at room temperature.
Cover the plates with foil to prevent the sensitive dyes from degrading
Aspirate off the secondary antibody solution followed by washing of the cells three times with PBST. Wash three times for 5 minutes each in dark.

### Counter Staining:

Incubate cells on 0.1-1 ug/ml Hoechst for 3 minutes at room temperature.

### Slide Preparation:

Make coverslips ready to be mounted onto microscope slides.
Take a clean slide and dispense one drop of antifade mounting medium by slowly from the pipette into the slide.
Carefully remove a coverslip from the well and allow excess wash to drip off and place the cells face down onto the slide.
Clear fingernail polish can be used to seal the coverslip and prevent it from drying out.
Visualized slide under microscope.

### Cobalt Chloride Hypoxia Assay

This assay is used to determine whether an intervention can reverse hypoxic-induced cell death brought about by cobalt chloride. For this assay, cells are pre-treated with CoCl₂ at 100 μM for 24 hours, followed by microscopic examination of cell morphology and viability.

### Results and Discussion

### Seahorse Bioenergetics Experiment

FIG. 29 shows the oxygen consumption rate (OCR) of OC-1 cells after 24 hours exposure to cisplatin (in FIG. 29, “PB” is probucol, “HA1” is HA-1, and “HA2” is HA-2). As can be seen, cisplatin has direct and concentration-dependent cytotoxic effects on OC-1 cells. Both analogues, HA-1 and HA-2, exerted significant and profound otoprotective effects with HA-2 being the most potent in reversing the cisplatin-induced cytotoxicity. This is evident from the enhanced OCR, which is indicative of greater respiration, and hence biological activity, taking place.

The experiment was expanded to evaluate full bioenergetic parameters after 48 hours exposure to cisplatin at the above concentrations.

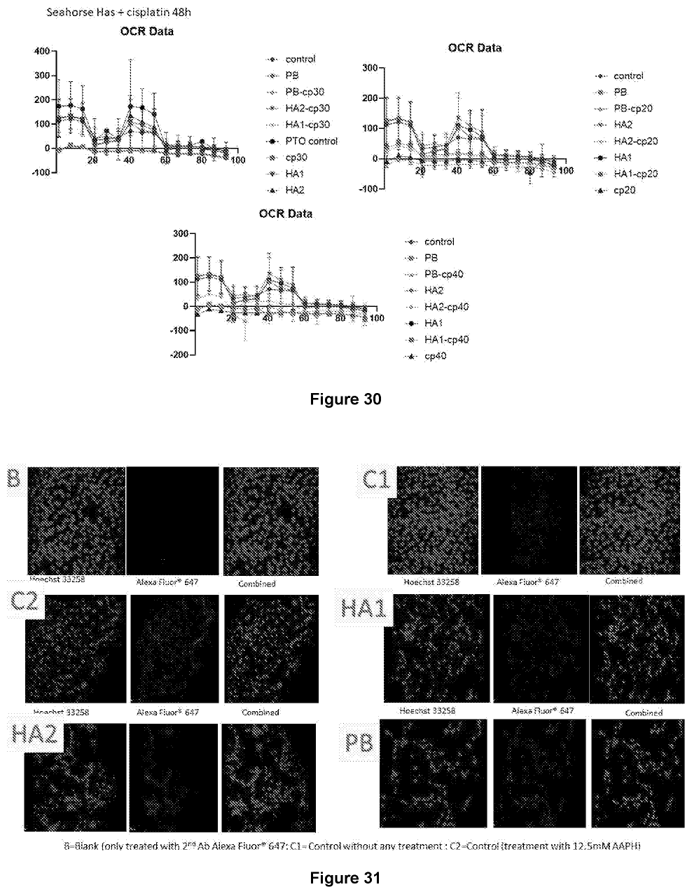

FIG. 30 shows OCR data of OC-1 cells after 48 hours exposure to cisplatin followed by treatment with HA-1 and HA-2. FIG. 30 has three graphical presentations as line graphs depicting cellular respiration (noted as OCR). The top left line graph shows the effects at cisplatin levels 30 μM, the top right line graph shows the effects at 20 μM, and the bottom line graph shows the effects at 40 μM. For each line graph, the higher the line sits above the X-axis, the better the effects of the treatment on the cells. For these line graphs, the lines sitting highest above the X-axis are HA-1, HA-2 and probucol, and the lowest lines (sitting almost flat) are those with cisplatin and not receiving treatments. As can be seen from FIG. 30, HA-1 and HA-2 were able to significantly reverse the cytotoxic effects of cisplatin as evidenced by the enhanced OCR, which in turn indicates greater respiration, metabolism and bioenergetics.

In order to explain the mechanisms and possible biological pathways implicated in the cytoprotective effects of HA-1 and HA-2, caspase assays were performed to determine whether the analogues are capable of suppressing apoptosis.

FIG. 31 shows the caspase assay results. FIG. 31 shows populations of cells in three sections (left, middle and right) for six sub-groups represented as B (blank: only treated with 2^{nd} Ab Alexa Fluor® 647), C1 (control: without any treatment), C2 (control: treatment with 12.5 mM AAPH), HA-1, HA-2 and PB. In each image, every dot represents a cell colony. For each sub-group, the left image has the greatest number of dots which represent very large numbers of cells as a total population, the centre image shows dots representing cells that are not living, and the right image shows dots that represent cell populations that are viable proportions of the total cell population (which would be comprised of living and dead cells that add up to give the “total”). So, the presence of more dots in the right image for each sub-group indicates the better the drug is working; HA-2 has the most dots in the right image, and is therefore the best at keeping cells alive under stressful, toxic conditions. This is followed by HA-1 and PB which show similar effects. All the treatment interventions had a positive impact, and the analogues HA-1 and HA-2 showed significant protection against programmed cell death brought about by caspase-3 activation pathways. HA-2 displayed the least amount of cell death due to cisplatin-induced oxidative stress and cytotoxicity.

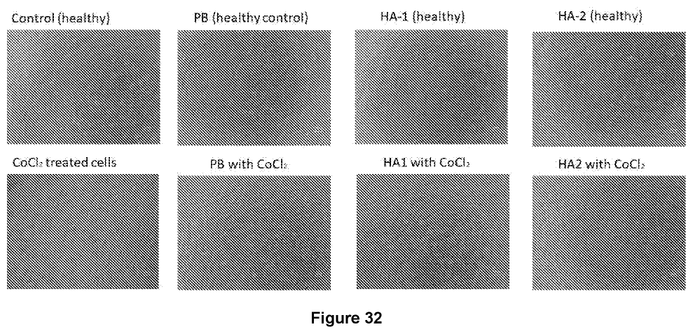

FIG. 32 shows cellular morphology and viability following cobalt chloride assays. This figure shows the populations of cells occupying space in each of the eight rectangles that make up the total figure. The more oval-shaped structures in each rectangle, the more living cells would be represented and, thus, the better would be the results as more living cells equate to better biological outcomes. Control (healthy) shown in the top far left rectangle had the most population of healthy cells (the most number of oval shaped-structures scattered throughout the rectangle) and this was similar for the entire top row of four rectangles (the top row had no stress induced onto the cells). The bottom row is where the discrepancies occur and the effects of the administered substances can be seen. The bottom far left rectangle shows the oval shapes have mostly disappeared and have been replaced by all sorts of mixed shapes which represent dead cells; however, the bottom row rectangles show that on the far right, HA-2 had retained all the oval shapes, and thus, protected the entire cell population against damage. This was followed by PB and HA-1 rectangles which showed the next most population of healthy, oval-shaped cells. CoCl₂ treated cells without any other intervention displayed profound loss of morphological characteristic traits with viability below 40±3%. Probucol-treated COCl₂ cells maintained normal morphology with viability of 70±2.5%, while HA-1 treated COCl₂ cells and HA-2 treated COCl₂ cells displayed optimal morphology with the most noticeable viability of 82±3.5% and 87±1.6% respectively.

1-27. (canceled)

28. A compound of Formula (I): 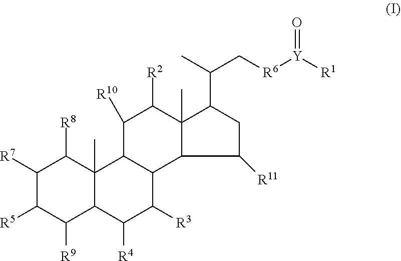
wherein:
R¹ is: 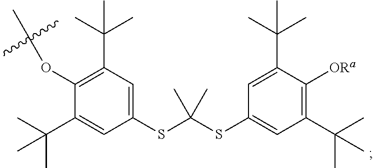
each R², R³, R⁴, R⁵, R⁷, R⁸, R⁹, R¹⁰ and R¹¹ is independently selected from H, substituted or unsubstituted C₁₋₃₀ acyloxy, substituted or unsubstituted benzoyloxy, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, substituted or unsubstituted aryl, substituted or unsubstituted arylalkyl, substituted or unsubstituted heteroaryl, an amino acid moiety, or GL, wherein GL is independently selected from OL, SL, PL₂, CL₃, or NL₂; wherein
when R², R³, R⁴, R⁵, R⁷, R⁸, R⁹, R¹⁰ or R¹¹ is substituted, the substituent is independently selected from OH, F, SH, ═O, ═S, Cl, Br, SC₁₋₆ alkyl, C₁₋₆ alkyl or C₁₋₆ alkoxy;
each L is independently selected from H, a metal ion, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, a substituted or unsubstituted benzyl radical, —CH₂CO₂H, or —(CH₂)₂SO₃H; wherein when L is substituted, the substituent is independently selected from OH, SH, ═O, ═S, F, Cl, Br, SC₁₋₆ alkyl, C₁₋₆ alkyl or C₁₋₆ alkoxy;
R⁶ is —(CH₂)ₙ— wherein n is 0 to 12, —CH₂C(═O)NHCH₂—, or —CH₂C(═O)NHCH₂CH₂—;
Y is —C— or —S(═O)—; and
R^{a} is —H or —C(═O)CH₂CH₂COOH;
or a stereoisomer thereof, or a pharmaceutically acceptable salt thereof.

29. A compound according to claim 28, wherein R^{a} is —H.

30. A compound according to claim 28, wherein R^{a} is —C(═O)CH₂CH₂COOH.

31. A compound according to claim 28, wherein each of R², R³, R⁴, R⁵, R⁷, R⁸, R⁹, R¹⁰ and R¹¹ is independently selected from H; substituted or unsubstituted C₁₋₃₀ acyloxy;
substituted or unsubstituted benzoyloxy; substituted or unsubstituted C₁₋₁₂ alkyl; substituted or unsubstituted C₂₋₁₂ alkenyl; substituted or unsubstituted C₂₋₁₂ alkynyl; substituted or unsubstituted C₃₋₈ cycloalkyl; substituted or unsubstituted C₆ aryl; substituted or unsubstituted heteroaryl; an amino acid moiety; or GL, wherein GL is independently selected from OL, SL, PL₂, CL₃, or NL₂.

32. A compound according to claim 28, wherein R², R³, R⁴, R⁵, R⁷, R⁸, R⁹, R¹⁰ and R¹¹ is independently selected from H; substituted or unsubstituted C₁₋₆ acyloxy; substituted or unsubstituted benzoyloxy; substituted or unsubstituted C₁₋₆ alkyl; substituted or unsubstituted C₂₋₆ alkenyl; substituted or unsubstituted C₂₋₆ alkynyl; substituted or unsubstituted C₃₋₈ cycloalkyl; substituted or unsubstituted phenyl; substituted or unsubstituted pyridyl; or OH.

33. A compound according to claim 28, wherein R², R³, R⁴, R⁵, R⁷, R⁸, R⁹, R¹⁰ or R¹¹ is independently selected from H, —OH and —OC(═O)R^{f}, wherein R^{f} is C₁₋₆ alkyl.

34. A compound according to claim 28, wherein R⁶ is selected from —(CH₂)ₙ— wherein n is 1, 2 or 3; —CH₂C(═O)NHCH₂—; or —CH₂C(═O)NHCH₂CH₂—.

35. A compound according to claim 28, wherein Y is —C—.

36. A compound according to claim 28, wherein the compound of Formula (I) is a compound of Formula (Ia): 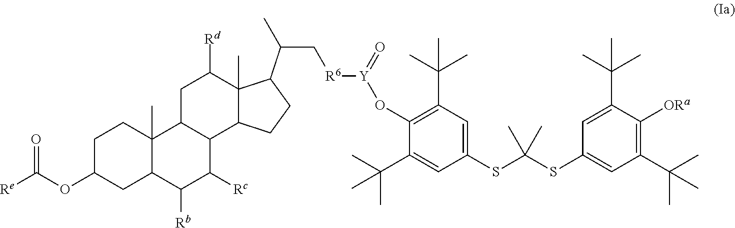
wherein:
R^{a} is —H or —C(═O)CH₂CH₂COOH;
R^{b} is selected from —H, —OH and —OC(═O)R^{f}, wherein R^{f} is C₁₋₆ alkyl;
R^{c} is selected from —H, —OH and —OC(═O)R^{f}, wherein R^{f} is C₁₋₆ alkyl;
R^{d} is selected from —H, —OH and —OC(═O)R^{f}, wherein R^{f} is C₁₋₆ alkyl;
R^{e} is selected from —C₁₋₆ alkyl;
R⁶ is —CH₂—, —CH₂C(═O)NHCH₂—, or —CH₂C(═O)NHCH₂CH₂—; and
Y is C or S(═O);
or a stereoisomer thereof, or a pharmaceutically acceptable salt thereof.

37. A compound according to claim 36, wherein R^{e} is selected from methyl (Me), ethyl (Et), or propyl (Pr).

38. A compound according to claim 36, wherein R⁶ is —CH₂—.

39. A compound according to claim 36, wherein Y is C.

40. A compound according to claim 36, wherein R^{a} is —H.

41. A compound according to claim 36, wherein R^{b} is H; R^{c} is H or OAc; and R^{d} is H.

42. A compound according to claim 36, wherein the compound is 2,6-di-tert-butyl-4-((2-((3,5-di-tert-butyl-4-hydroxyphenyl)thio)propan-2-yl)thio)phenyl (4R)-4-[(3R,5R,8R,9S,10S,13R,14S,17R)-3-acetoxy-10,13-dimethyl-2,3,4,5,6,7,8,9,11,12,14,15,16,17-tetradecahydro-1H-cyclopenta[a]phenanthren-17-yl]pentanoate: 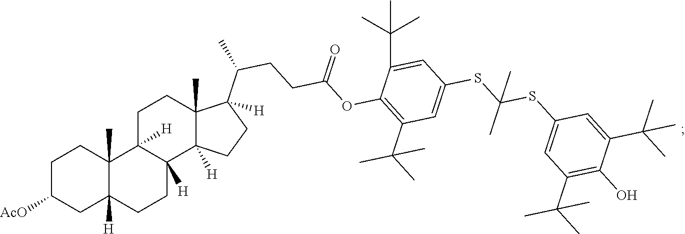
or a stereoisomer thereof, or a pharmaceutically acceptable salt thereof.

43. A pharmaceutical composition comprising a compound according to claim 28, and a pharmaceutically acceptable carrier, adjuvant or diluent.

44. A method of treating or preventing a neurological disorder or cognitive decline associated with a neurological disorder in a subject, the method comprising administering to the subject an effective amount of a compound according to claim 28.

45. A method according to claim 44, wherein the neurological disorder is Alzheimer's disease, dementia caused by Alzheimer's disease, Parkinson's disease, amyotrophic lateral sclerosis (motor neurone disease), or multiple sclerosis; and/or the cognitive decline is memory loss.

46. A method for preventing, reducing or treating the incidence and/or severity of a disorder caused by stress-induced cellular damage in the middle or inner ear of a subject, or for preventing, reducing or inhibiting hair cell degeneration or hair cell death in a subject, the method comprising administering to the subject a therapeutically effective amount of a compound according to claim 28.

47. A method according to claim 46, wherein the disorder caused by stress-induced cellular damage in the middle or inner ear is a vestibular disorder or hearing impairment.
